(19) United States
(12) Patent Application Publication
(10) Pub. No.: US 2014/0356283 A1
(43) Pub. Date: Dec. 4, 2014

(54) Probes, Methods of Making Probes, and Methods of Use

(71) Applicant: The Board of Trustees of the Leland Stanford Junior University, Palo Alto, CA (US)

(72) Inventors: Zhen Cheng, Mountain View, CA (US); Quli Fan, Nanjing (CN); Kai Cheng, Sunnyvale, CA (US); Xiang Hu, Wuhan (CN)

(21) Appl. No.: 14/288,441

(22) Filed: May 28, 2014

Related U.S. Application Data
(60) Provisional application No. 61/830,753, filed on Jun. 4, 2013.

(51) Int. Cl. A61K 49/22; A61K 49/12; A61K 49/04; A61K 49/18

(52) U.S. Cl. CPC A61K 49/225; A61K 49/1824; A61K 49/126; A61K 49/04
USPC 424/1.65; 428/402; 424/9.1; 424/9.3; 548/455; 435/29

(57) ABSTRACT

Embodiments of the present disclosure provide for water soluble polyethylene glycol (PEG)-polymeric melanin (PEG-melanin) nanoparticles, methods of using the PEG-melanin nanoparticle, methods of making the PEG-melanin nanoparticle, methods of imaging a diseases or condition (e.g., pre-cancerous tissue, cancer, or a tumor), and the like. Embodiments of the present disclosure can be used in multimodal imaging (e.g., photoacoustic, nuclear, magnetic, fluorescent, and the like).

### CROSS-REFERENCE TO RELATED APPLICATION

This application claims priority to U.S. provisional application entitled, “PROBES, METHODS OF MAKING PROBES, AND METHODS OF USE” having Ser. No. 61/830,753, filed on Jun. 4, 2013, which is entirely incorporated herein by reference.

### FEDERAL SPONSORSHIP

This invention was made with Government support under Contract/Grant No. DE-SC0008397, awarded by Department of Energy. The Government has certain rights in this invention.

### BACKGROUND

Naturally produced biopolymers in living organisms play crucial roles in materials discovery and development. They have inspired scientists to synthesize novel biomaterials through mimicking Mother Nature, or they can further serve as templates and building blocks to prepare new generations of biocompatible, bioregenerative, or biodegradable materials for biomedical applications.

Multimodal imaging combines different modalities together to provide complementary information and achieve synergistic advantages over any single modality alone. It has emerged as a very promising strategy for pre-clinical research and clinical applications. One major challenge of multimodal imaging is to develop an efficient platform to load various components with individual contrast properties together while maintaining compact size, good biocompatibility and targeting capability. A variety of nanomaterials have been explored for multimodal imaging. However, there is a need to produce other types of multimodality imaging probes to address various diseases and conditions.

### SUMMARY

Embodiments of the present disclosure provide for water soluble polyethylene glycol (PEG)-polymeric melanin (PEG-melanin) nanoparticles, methods of using the PEG-melanin nanoparticle, methods of making the PEG-melanin nanoparticle, methods of imaging a diseases or condition (e.g., pre-cancerous tissue, cancer, or a tumor), and the like. Embodiments of the present disclosure can be used in multimodal imaging (e.g., photoacoustic, nuclear, magnetic, fluorescent, and the like).

An embodiment of the present disclosure includes a composition, among others, that includes: a water soluble polyethylene glycol (PEG)-polymeric melanin (PEG-melanin) nanoparticle, wherein the PEG is attached to the surface of the polymeric melanin nanoparticle core, wherein the polymeric melanin nanoparticle core has a diameter of about 3 to 10 nm. An embodiment of the PEG-melanin includes a MRI agent, a PET agent, and/or SPECT agent.

An embodiment of the present disclosure includes a method of imaging a disease, among others, that includes: exposing a subject to an imaging device, wherein a PEG-melanin nanoparticle is introduced to a subject, wherein PEG-melanin nanoparticle is a water soluble PEG-melanin nanoparticle, wherein the PEG is attached to the surface of the polymeric melanin nanoparticle core, wherein the polymeric melanin nanoparticle core has a diameter of about 3 to 10 nm; and detecting the PEG-melanin nanoparticle, wherein the location of the PEG-melanin nanoparticle correlates to the location of the disease.

An embodiment of the present disclosure includes a pharmaceutical composition, among others, that includes: a pharmaceutical carrier and an effective amount of a PEG-melanin nanoparticle, wherein PEG-melanin nanoparticle is a water soluble PEG-melanin nanoparticle, wherein the PEG is attached to the surface of the polymeric melanin nanoparticle core, wherein the polymeric melanin nanoparticle core has a diameter of about 3 to 10 nm.

An embodiment of the present disclosure includes a method of making a PEG-melanin nanoparticle, among others, that includes: dissolving melanin in a basic aqueous solution; adjusting the pH to about 7 under sonication to form melanin nanoparticles; adjusting the pH to about 10; and adding PEG precursor compounds to the solution to form PEG-melanin nanoparticles, wherein the PEG is attached to the surface of a polymeric melanin nanoparticle core, wherein the polymeric melanin nanoparticle core has a diameter of about 3 to 10 nm.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1.1A illustrates Scheme 1 showing the preparation route for water-soluble melanin NPs, PEG-melanin NPs and NOTA-PEG-melanin NPs. FIG. 1B illustrates TEM of WS-melanin NPs (left) and PEG-melanin NPs (right), scale bar=20 nm.

FIG. 1.2 illustrates the hydrodynamic size distribution graphs of WS-melanin NPs (top) and PEG-melanin NPs (bottom).

FIG. 1.3 illustrates the Zeta potentials of WS-melanin NPs (top) and PEG-melanin NPs (bottom).

FIG. 1.4, from left to right, illustrates (1) pristine melanin in H₂O, (2) melanin neutralized without sonication in H₂O, (3) freeze-dried WS-melanin, (4) freeze-dried WS-melanin redissolved in H₂O, (5) freeze-dried PEG-melanin, and (6) freeze-dried melanin-PEG redissolved in H₂O.

FIG. 1.5 illustrates FT-IR spectra of pristine melanin, WS-melanin and PEG-melanin.

FIG. 1.6 illustrates ¹H NMR spectra of WS-melanin and PEG-melanin in D₂O.

FIG. 1.7 illustrates the plot of the relationship between the weight ratio of the product composition (PEG:melanin) with feed composition (PEG:melanin).

FIG. 1.8 illustrates the UV-vis-NIR absorption spectra of WS-melanin nanoparticles and PEG-melanin NPs.

FIG. 1.9 illustrates serum stability of melanin NPs. WS-melanin and PEG-melanin particles were exposed to 10% serum and their optical absorbance was measured over a period of 24 h. Control solutions included SWNT-QSY-RGD in PBS and SWNT-ICG-RGD in PBS only. All solutions showed steady optical absorption within ±2% over the 24 h period.

FIG. 1.10 illustrates the photobleaching of melanin NPs. WS-melanin and PEG-melanin samples (n=3 for each) were exposed to increasing durations of 680 nm laser light, at power density of 8 mJ/cm². After 60 min of laser exposure, the optical absorption of all the melanin particles was reduced by ˜3%.

FIG. 1.11 illustrates the MTT assay using NIH-3T3 cells with WS-melanin and PEG-melanin NP concentration 0.2, 0.02, and 0.002 mg/mL after 24 h incubation at 37° C.

FIG. 1.12 illustrates the photoacoustic signal produced by PEG-melanin NPs was observed to be linearly dependent on its concentration (R²=0.995).

FIG. 1.13 illustrates the photoacoustic detection of PEG-melanin NPs in living mice. Mice were injected subcutaneously with PEG-melanin NPs at concentrations of 0, 0.4, 0.8, 1.6, 3.2, 6.4 mg/mL. One vertical slice in the 3D photoacoustic image (light grey) was overlaid on the corresponding slice in the ultrasound image (grey). The skin is visible in the ultrasound images, and the photoacoustic images show the PEG-melanin NPs. The dotted lines on the images identify the edges of each inclusion.

FIG. 1.14 illustrates the photoacoustic signal from each inclusion was calculated. The background level represents the endogenous signal measured from tissues. The error bars represent standard error (n=3). The linear regression is calculated on the five most concentrated inclusions (R²=0.998).

FIG. 1.15 illustrates the ultrasonic (US), photoacoustic (PA) and their overlaying imaging of PEG-melanin NPs in mouse liver.

FIG. 1.16 illustrates the photoacoustic signal strengths of the skin and the liver after Mice were injected with PEG-melanin NPs respectively.

FIG. 1.17 illustrates in vitro mouse serum stability study of PEG-melanin NPs.

FIG. 1.18 illustrates the biodistribution of ⁶⁴Cu-NOTA-PEG-melanin NPs in mice 1 day after injection.

FIG. 1.19 illustrates the biodistribution of ⁶⁴Cu-NOTA-PEG-melanin NPs in mice 2 h, 4 h, 12 h, and 24 h after injection.

FIG. 1.20 illustrates the PET of ⁶⁴Cu-RGD-melanin NPs for subcutaneous tumor in mice after 24 h injection.

FIG. 1.21 illustrates the MRI of Fe³⁺-RGD-melanin NPs for subcutaneous tumor in mice after 24 h injection.

FIG. 1.22 illustrates the inhibition of U87-MG cell growth in the presence of drug/melanin at 0, 10, 50, 100 μg/mL) after 24 h incubation.

FIG. 2.1 illustrates multimodality molecular imaging of MNPs. The melanin granules were first dissolved in 0.1N NaOH aqueous solution, and then neutralized under sonication to obtain melanin nanoparticles in high water monodispersity and homogeneity. After PEG surface-modification, RGD was further attached to the MNP for tumor targeting. Then Fe³⁺ and/or ⁶⁴Cu²⁺ were chelated to the obtained MNPs for PAI/MRI/PET multimodal imaging.

FIG. 2.2 illustrates the characterization of physical properties of MNPs. FIG. 2.2A, from left to right: illustrates pictures of (1) pristine melanin granule in H₂O, (2) melanin neutralized without sonication in H₂O, (3) freeze-dried PWS-MNP, (4) freeze-dried PWS-MNP redissolved in PBS (pH=7.4), (5) freeze-dried PEG-MNP, (6) freeze-dried PEG-MNP redissolved in PBS (pH=7.4). FIG. 2.2B illustrate TEM of PWS-MNP (left) and PEG-MNP (right), scale bar=20 nm. FIG. 2.2C illustrate the plot of the relationship between the number of metal ions attached on one MNP with feed ratio (Wᵢₒₙₛ: W_{MNP}). FIG. 2.2D illustrates in vitro mouse serum stability study of metal ion-chelated MNPs.

FIG. 2.3 illustrates in vitro and in vivo study of PAI of MNPs. FIG. 2.3A illustrate the photoacoustic signal produced by PEG-MNPs at concentrations of 0.625, 1.25, 2.5, 5.0, 10, and 20 μM, and it was observed to be linearly dependent on its concentration (R²=0.995). FIG. 2.3B illustrates the photoacoustic detection of PEG-MNP in living mice. Mice were injected subcutaneously with PEG-MNP at concentrations of 0, 5, 10 (from left to right in top row), and 20, 40, 80 (from left to right in bottom row) μM. One vertical slice in the photoacoustic image (red) was overlaid on the corresponding slice in the ultrasound image (grey). FIG. 2.3C illustrates the photoacoustic signal from each inclusion was calculated. The background level represents the endogenous signal measured from tissues. The linear regression is calculated on the five most concentrated inclusions (R²=0.998). FIG. 2.3D illustrates the overlaying of ultrasonic (grey) and photoacoustic (light grey) imaging of U87MG tumor before and after tail-vein injection of 250 μL of 200 μM PEG-MNP and RGD-PEG-MNP in living mouse. FIG. 2.3E illustrates the quantitative analysis of U87MG tumor images obtained from PAI after tail-vein injection with RGD-PEG-MNP and PEG-MNP at 4 h.

FIG. 2.4 illustrates in vitro and in vivo study of MRI of Fe³⁺-chelated MNPs. FIG. 2.4A illustrates T₁ relaxation rates (1/T₁, s⁻¹) as a function of Fe-RGD-PEG-MNP (mM) in agarose gel (1.0 T, 25° C.). FIG. 2.4N illustrates MRI images of U87MG cells incubated with three concentrations of Fe-RGD-PEG-MNP (top row) and Fe-PEG-MNP (bottom row) for 4 h. c, MRI detection of Fe-RGD-PEG-MNP s in living mice. Mice were injected subcutaneously with Fe-RGD-PEG-MNPs at concentrations of 0, 1.25, 2.5 (from left to right in upper layer), and 5, 10, 20 (from left to right in bottom layer) μM. FIG. 2.4D illustrates the quantitative analysis of U87MG tumor images obtained from MRI after tail-vein injection with Fe-RGD-PEG-MNP and Fe-PEG-MNP at 4 h. Fe-RGD-PEG-MNP displays relatively higher tumor/muscle ration than that Fe-PEG-MNP. FIG. 2.4E illustrates MRI images of U87MG tumors before and after tail-vein injection of 250 μL of 200 μM PEG-MNP and RGD-PEG-MNP in living mouse. Top row shows black and weight images, and bottom row shows the pseudo-colored images.

FIG. 2.5 illustrates in vitro and in vivo study of PET of ⁶⁴Cu-labelled MNPs. FIG. 2.5A illustrates the uptake of ⁶⁴Cu-PEG-MNP, ⁶⁴Cu-RGD-PEG-MNP with and without blocking in U87MG cells at 37° C. for 1, 2 and 4 h incubation. All results, expressed as percentage of cellular uptake, are mean of triplicate measurements±SD. FIG. 2.5B illustrates representative decay-corrected coronal (top) and transaxial (bottom) small animal PET images of U87MG tumors acquired at 2, 4 and 24 h after tail vein injection of ⁶⁴Cu-RGD-PEG-MNP (left three images) and ⁶⁴Cu-PEG-MNP (right three images). FIG. 2.5C illustrates the biodistribution of ⁶⁴Cu-RGD-PEG-MNP (left) and ⁶⁴Cu-PEG-MNP (right) in mice 2 h, 4 h, 12 h, and 24 h after injection. The radioactive signal from each organ was calculated using a region of interest drawn over the whole organ region. FIG. 2.5D illustrates the quantitative analysis of U87MG tumor images obtained from PET after tail-vein injection with ⁶⁴Cu-RGD-PEG-MNP and ⁶⁴Cu-PEG-MNP at 4 h.

FIG. 2.6 illustrates the in vivo multimodality imaging of tumor bearing mice with PAI, MRI and PET. FIG. 2.6A illustrates the photographic images of U87MG tumor bearing mice. FIG. 2.6B illustrates the overlaying of ultrasonic (grey) and photoacoustic (red) imaging of U87MG tumor before and after tail-vein injection of ⁶⁴Cu—Fe-RGD-PEG-MNP in living mouse. FIG. 2.6C illustrates the representative decay-corrected coronal (top) and transaxial (bottom) small animal PET images of U87MG tumors acquired at 2, 4 and 24 h after tail vein injection of ⁶⁴Cu—Fe-RGD-PEG-MNP. FIG. 2.6D illustrates MRI images of U87MG tumor before and after tail-vein injection of ⁶⁴Cu—Fe-RGD-PEG-MNP in living mouse. Top row shows black and weight images, and bottom row shows the pseudo-colored images.

FIG. 2.7A illustrates zeta potentials of PWS-MNP (top) and PEG-MNP (bottom); while FIG. 2.7B illustrates hydrodynamic size distribution graphs of PWS-MNP (top) and PEG-MNP (bottom).

FIG. 2.8A illustrates FT-IR spectra of pristine melanin granule, PWS-MNP and PEG-MNP. FIG. 2.8B illustrates ¹H NMR spectra of PWS-MNP and PEG-MNP in D₂O.

FIG. 2.9A illustrates a plot of the relationship between the weight ratio of the product composition (PEG:PWS-MNP) and the feed ratio (W_{PEG}: W_{PWS-MNP}). FIG. 2.9B illustrates the UV-vis-NIR absorption spectra of PWS-MNP and PEG-MNP.

FIG. 2.10A illustrates the hydrodynamic size distribution graphs of RGD-PEG-MNP (top), Fe-PEG-MNP (middle), and Fe-RGD-PEG-MNP (bottom). FIG. 2.10B illustrates the zeta potentials of RGD-PEG-MNP (top), Fe-PEG-MNP (middle), and Fe-RGD-PEG-MNP (bottom).

FIG. 2.11, from left to right, illustrates pictures of (1) 1 mL of 20 μM PWS-MNP aqueous solution after adding 0.2 mL of 10 mM FeCl₃, (2) 1 mL of 20 μM PWS-MNP aqueous solution after adding 0.2 mL of 10 mM CuCl₂, (3) 1 mL of 20 μM PEG-MNP aqueous solution after adding 0.2 mL of 10 mM FeCl₃, (4) 1 mL of 20 μM PEG-MNP aqueous solution after adding 0.2 mL of 10 mM CuCl₂. It was showed that PEG-encapsulation will hamper the formation of precipitation of MNPs after adding metal ions.

FIG. 2.12 illustrates photobleaching of MNPs. RGD-PEG-MNP and PEG-MNP samples (n=3 for each) were exposed to increasing durations of 680 nm laser light, at power density of 8 mJ/cm². After 60 min of laser exposure, the optical absorption of all the MNPs was reduced by ˜3%.

FIG. 2.13 illustrates MTT assay using NIH-3T3 cells with MNP concentration 0.2, 0.5, 1 and 2 μM after 24 h incubation at 37° C.

FIG. 2.14 illustrates T₁-weighted MRI images (1.0 T, spin-echo sequence: repetition time TR=700 ms, echo time TE=5.5 ms) of Fe-RGD-PEG-MNP with different concentration.

FIG. 2.15 illustrates in vitro mouse serum and PBS stability study of ⁶⁴Cu-RGD-PEG-MNP and ⁶⁴Cu-PEG-MNP. After 24 h incubation, only ˜3% ⁶⁴Cu was released from the MNPs.

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of imaging, chemistry, synthetic organic chemistry, biochemistry, biology, molecular biology, microbiology, and the like, which are within the skill of the art. Such techniques are explained fully in the literature.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

### DEFINITIONS

In describing and claiming the disclosed subject matter, the following terminology will be used in accordance with the definitions set forth below.

Unless otherwise defined, all terms of art, notations and other scientific terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art. As appropriate, procedures involving the use of commercially available kits and reagents are generally carried out in accordance with manufacturer defined protocols and/or parameters unless otherwise noted.

The term “detectable” refers to the ability to detect a signal over the background signal.

The term “acoustic detectable signal” is a signal derived from a nanoparticle core that absorbs light and converts absorbed energy into thermal energy that causes generation of acoustic signal through a process of thermal expansion. The acoustic detectable signal is detectable and distinguishable from other background acoustic signals that are generated from the subject or sample. In other words, there is a measurable and statistically significant difference (e.g., a statistically significant difference is enough of a difference to distinguish among the acoustic detectable signal and the background, such as about 0.1%, 1%, 3%, 5%, 10%, 15%, 20%, 25%, 30%, or 40% or more difference between the acoustic detectable signal and the background) between acoustic detectable signal and the background. Standards and/or calibration curves can be used to determine the relative intensity of the acoustic detectable signal and/or the background. Similarly, “MRI detectable signal” or “PET (or SPECT) detectable signal” can be derived from an appropriate agent attached to the nanoparticle, and have a similar meaning.

The term “acoustic signal” refers to a sound wave produced by one of several processes, methods, interactions, or the like (including light absorption) that provides a signal that can then be detected and quantitated with regards to its frequency and/or amplitude. The acoustic signal can be generated from one or more nanoparticle cores of the probes of the present disclosure. In an embodiment, the acoustic signal may need to be sum of each of the individual photoacoustic signals. In an embodiment, the acoustic signal can be generated from a summation, an integration, or other mathematical process, formula, or algorithm, where the acoustic signal is from one or more probes. In an embodiment, the summation, the integration, or other mathematical process, formula, or algorithm can be used to generate the acoustic signal so that the acoustic signal can be distinguished from background noise and the like. It should be noted that signals other than the acoustic signal can be processed or obtained is a similar manner as that of the acoustic signal.

The acoustic signal or acoustic energy can be detected and quantified in real time using an appropriate detection system. One possible system is described in the following references: Journal of Biomedical Optics-March/April 2006-Volume 11, Issue 2, 024015, Optics Letters, Vol. 30, Issue 5, pp. 507-509, each of which are included herein by reference. In an embodiment, the acoustic energy detection system can includes a 5 MHz focused transducer (25.5 mm focal length, 4 MHz bandwidth, F number of 2.0, depth of focus of 6.5 mm, lateral resolution of 600 μm, and axial resolution of 380 μm. A3095-SU-F-24.5-MM-PTF, Panametrics), which can be used to acquire both pulse-echo and photoacoustic images. In addition, high resolution ultrasound images can also be simultaneously acquired using a 25 MHz focused transducer (27 mm focal length, 12 MHz bandwidth, F number of 4.2, depth of focus of 7.5 mm, lateral resolution of 250 μm, and axial resolution of 124 μm. V324-SU-25.5-MM, Panametrics). Other detection strategies including capacitive micromachined ultrasonic transducers (CMUT) arrays can also be used to detect the acoustic signal.

The term “illuminating” as used herein refers to the application of a light source, including near-infrared (NIR), visible light, including laser light capable of exciting melanin or melanin-PEG nanoparticles of the present disclosure.

The term “magnetic resonance imaging (MRI)” as used herein refers to a medical imaging technique most commonly used in radiology to visualize the structure and function of the body. It provides detailed images of the body in any plane. MRI uses no ionizing radiation, but uses a powerful magnetic field to align the nuclear magnetization of (usually) hydrogen atoms in water in the body. Radiofrequency fields are used to systematically alter the alignment of this magnetization, causing the hydrogen nuclei to produce a rotating magnetic field detectable by the scanner. This signal can be manipulated by additional magnetic fields to build up enough information to construct an image of the body. When a subject lies in a scanner, the hydrogen nuclei (i.e., protons) found in abundance in an animal body in water molecules, align with the strong main magnetic field. A second electromagnetic field that oscillates at radiofrequencies and is perpendicular to the main field, is then pulsed to push a proportion of the protons out of alignment with the main field. These protons then drift back into alignment with the main field, emitting a detectable radiofrequency signal as they do so. Since protons in different tissues of the body (e.g., fat versus muscle) realign at different speeds, the different structures of the body can be revealed. Contrast agents may be injected intravenously to enhance the appearance of blood vessels, tumors or inflammation. MRI is used to image every part of the body, but is particularly useful in neurological conditions, disorders of the muscles and joints, for evaluating tumors and showing abnormalities in the heart and blood vessels.

The term “positive contrast” as used herein refers to the differences in the observed MRI image between that of a targeted tissue site that generates a greater detectable signal intensity than the intensity of a signal generated in a surrounding tissue.

The term “negative contrast” as used herein refers to the difference in the observed MRI image between that of a targeted tissue site that has a lower detectable signal intensity than the intensity of a signal generated in a surrounding tissue.

The term “in vivo imaging” as used herein refers to methods or processes in which the structural, functional, or physiological state of a living being is examinable without the need for a life ending sacrifice.

The term “non-invasive in vivo imaging” as used herein refers to methods or processes in which the structural, functional, or physiological state of a being is examinable by remote physical probing without the need for breaching the physical integrity of the outer (skin) or inner (accessible orifices) surfaces of the body.

The term “sample” can refer to a tissue sample, cell sample, a fluid sample, and the like. The sample may be taken from a subject. The tissue sample can include brain, hair (including roots), buccal swabs, blood, saliva, semen, muscle, or from any internal organs, or cancer, precancerous, or tumor cells associated with any one of these. The fluid may be, but is not limited to, urine, blood, ascites, pleural fluid, spinal fluid, and the like. The body tissue can include, but is not limited to, brain, skin, muscle, endometrial, uterine, and cervical tissue or cancer, precancerous, or tumor cells associated with any one of these. In an embodiment, the body tissue is brain tissue or a brain tumor or cancer.

The term “administration” refers to introducing a probe of the present disclosure into a subject. One preferred route of administration of the compound is oral administration. Another preferred route is intravenous administration. However, any route of administration, such as topical, subcutaneous, peritoneal, intraarterial, inhalation, vaginal, rectal, nasal, introduction into the cerebrospinal fluid, or instillation into body compartments can be used.

As used herein, the term “host,” “subject,” or “patient,” includes humans, mammals (e.g., mice, rats, pigs, cats, dogs, and horses), and poultry. Typical hosts to which compounds of the present disclosure may be administered will be mammals, particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like. The term “living subject” refers to a subject noted above or another organism that is alive. The term “living subject” refers to the entire subject or organism and not just a part excised (e.g., a liver or other organ) from the living subject.

“Cancer”, as used herein, shall be given its ordinary meaning, as a general term for diseases in which abnormal cells divide without control. In particular, cancer refers to melanin related cancer. Cancer cells can invade nearby tissues and can spread through the bloodstream and lymphatic system to other parts of the body.

There are several main types of cancer, for example, carcinoma is cancer that begins in the skin or in tissues that line or cover internal organs. Sarcoma is cancer that begins in bone, cartilage, fat, muscle, blood vessels, or other connective or supportive tissue. Leukemia is cancer that starts in blood-forming tissue such as the bone marrow, and causes large numbers of abnormal blood cells to be produced and enter the bloodstream. Lymphoma is cancer that begins in the cells of the immune system.

When normal cells lose their ability to behave as a specified, controlled and coordinated unit, a tumor is formed. Generally, a solid tumor is an abnormal mass of tissue that usually does not contain cysts or liquid areas (some brain tumors do have cysts and central necrotic areas filled with liquid). A single tumor may even have different populations of cells within it, with differing processes that have gone awry. Solid tumors may be benign (not cancerous), or malignant (cancerous). Different types of solid tumors are named for the type of cells that form them. Examples of solid tumors are sarcomas, carcinomas, and lymphomas. Leukemias (cancers of the blood) generally do not form solid tumors.

Representative cancers include, but are not limited to, bladder cancer, breast cancer, colorectal cancer, endometrial cancer, head and neck cancer, leukemia, lung cancer, lymphoma, melanoma, non-small-cell lung cancer, ovarian cancer, prostate cancer, testicular cancer, uterine cancer, cervical cancer, thyroid cancer, gastric cancer, brain stem glioma, cerebellar astrocytoma, cerebral astrocytoma, glioblastoma, ependymoma, Ewing's sarcoma family of tumors, germ cell tumor, extracranial cancer, Hodgkin's disease, leukemia, acute lymphoblastic leukemia, acute myeloid leukemia, liver cancer, medulloblastoma, neuroblastoma, brain tumors generally, non-Hodgkin's lymphoma, osteosarcoma, malignant fibrous histiocytoma of bone, retinoblastoma, rhabdomyosarcoma, soft tissue sarcomas generally, supratentorial primitive neuroectodermal and pineal tumors, visual pathway and hypothalamic glioma, Wilms' tumor, acute lymphocytic leukemia, adult acute myeloid leukemia, adult non-Hodgkin's lymphoma, chronic lymphocytic leukemia, chronic myeloid leukemia, esophageal cancer, hairy cell leukemia, kidney cancer, multiple myeloma, oral cancer, pancreatic cancer, primary central nervous system lymphoma, skin cancer, small-cell lung cancer, among others.

A tumor can be classified as malignant or benign. In both cases, there is an abnormal aggregation and proliferation of cells. In the case of a malignant tumor, these cells behave more aggressively, acquiring properties of increased invasiveness. Ultimately, the tumor cells may even gain the ability to break away from the microscopic environment in which they originated, spread to another area of the body (with a very different environment, not normally conducive to their growth), and continue their rapid growth and division in this new location. This is called metastasis. Once malignant cells have metastasized, achieving a cure is more difficult.

In an embodiment, cancer refers to malignant melanoma.

The phrase “melanin related diseases” can refer to malignant melanoma, hyperpigmentation, and other diseases that accompanied with change of melanin content.

### General Discussion

Embodiments of the present disclosure provide for water soluble polyethylene glycol (PEG)-polymeric melanin (PEG-melanin) nanoparticles, methods of using the PEG-melanin nanoparticle, methods of making the PEG-melanin nanoparticle, methods of imaging a diseases or condition (e.g., pre-cancerous tissue, cancer, or a tumor), and the like. Embodiments of the present disclosure can be used in multimodal imaging (e.g., photoacoustic, nuclear, magnetic, fluorescent, and the like). Embodiments of the present disclosure can be used to image, detect, study, monitor, and/or evaluate, a condition or disease such as pre-cancerous tissue, cancer, or a tumor, specifically, a melanin related cancer such as malignant melanoma. In addition, embodiments of the present disclosure can be used to deliver therapeutic agents such as small molecule drugs.

Embodiments of the present disclosure illustrate the successful transferring an important molecular target, melanin, into a novel multimodality imaging nanoplatform (PEG-melanin nanoparticles). Melanin is abundantly expressed in melanotic melanomas and thus has been actively studied as a target for melanoma imaging. In an embodiment, the PEG-melanin nanoparticles showed unique photoacoustic property and natural binding ability with metal ions (for example, ⁶⁴Cu²⁺, Fe³⁺). Therefore the PEG-melanin nanoparticles can serve not only as a photoacoustic contrast agent, but also as a nanoplatform for positron emission tomography (PET) and magnetic resonance imaging (MRI). Traditional passive nanoplatforms require complicated and time-consuming processes for pre-building reporting moieties or chemical modifications using active groups to integrate different contrast properties into one entity. In comparison, utilizing functional biomarker melanin can greatly simplify the building process. The PEG-melanin nanoparticles are the first natural biomarker-transferred active platform for multimodality imaging. Embodiments of the present disclosure are of interest because such an endogenous agent with native photoacoustic signals and strong chelating properties with metal ions can act as an active platform to simplify the assembling of different imaging moieties. The PEG-melanin nanoparticles can be easily modified with biomolecules for targeted tumor multimodality imaging, and it showed excellent in vivo tumor imaging properties. Embodiments of the PEG-melanin nanoparticles demonstrate the high potential of endogenous materials with multifunctions as nanoplatforms for molecular theranostics and clinical translation.

An embodiment of a water soluble polyethylene glycol (PEG)-polymeric melanin (PEG-melanin) nanoparticle can include a PEG attached to the surface of the polymeric melanin nanoparticle core. The polymeric melanin nanoparticle core can act as a photoacoustic probe with a detectable photoacoustic signal. In an embodiment, a photoacoustic signal can be generated by directing optical energy (e.g., a laser) toward the polymeric melanin nanoparticle core and the core absorbing the optical energy and converting the energy into thermal energy to produce an acoustic signal.

In an embodiment, the polymeric melanin nanoparticle core can have a diameter of about 3 to 6, about 7 to 10 nm, about 10 to 30 nm, about 20 to 50 nm, or about 40 to 100 nm. In an embodiment, the PEG-melanin nanoparticle can have a diameter of about 4 to 10 nm, about 10 to 15 nm, about 15 to 40 nm, about 30 to 70 nm, or about 50 to 150 nm.

In an embodiment, the polymeric melanin core can be made of natural or synthetic melanin. In an embodiment, the synthetic melanin can be made be derived from tyrosine. In an embodiment, the polymeric melanin can have a molecular weight of about 10,000 kDa to 3,000,000 kDa. Additional details regarding the polymeric melanin core are described in Example 1.

In an embodiment, the PEG can be bonded (e.g., directly or indirectly) to the polymeric melanin core. For example, the PEG can be bonded to the polymeric melanin core via thiol or amine groups on the PEG. In an embodiment, the PEG-melanin nanoparticle can include 5 to 50 PEGs. In an embodiment, the PEG can be a linear PEG, a multi-arm PEG, a branched PEG, and combinations thereof. The molecular weight of the PEG can be about 1 kDa to 100 kDa, about 1 kDa to 50 kDa, about 1 kDa to 40 kDa, about 1 kDa to 30 kDa, about 1 kDa to 20 kDa, about 1 kDa to 12 kDa, about 1 kDa to 10 kDa, and about 1 kDa to 8 kDa. When used in reference to PEG moieties, the word “about” indicates an approximate average molecular weight and reflects the fact that there will normally be a certain molecular weight distribution in a given polymer preparation.

Alternatively, one or more PEGs can be replaced with n-MEG, poly(vinyl alcohol) (PVA), poly(acrylic acid) (PAA), poly(propylene furmarate-co-ethylenee glycol) (P(PF-co-EG)), polyacrylamide, polypeptides, poly-N-substituted glycine oligomers (polypeptoids), and the like, as well as with naturally derived polymers normally include hyaluronic acid (HA), alginate, chitosan, agarose, collagen, fibrin, gelatin, dextran, and any combination thereof, as well as derivatives of each of these.

In an embodiment, the PEG-melanin nanoparticle can include a MRI agent, a PET or SPECT agent, or a combination thereof. The signal from each agent can be detected using a separate detection system or one or two systems can be combined to detect multiple agent types. A detection device can be used to image (e.g., photoacoustic device, MRI device, fluorescent detection system, nuclear detection system, and the like) the subject. In each of these, the signal (e.g., MRI signal, fluorescent signal, nuclear imaging signal, etc.) can be used to correlate the position of the disease within the subject. Additional details regarding detection of the signals are provided in the Examples.

In an embodiment, the PEG-melanin nanoparticle can include a MRI agent that has a detectable MRI signal. In an embodiment, the amount or number of MRI agents disposed (e.g., directly or indirectly) on the PEG-melanin nanoparticle can be about 1 to 50 MRI agents. In an embodiment all or a portion of the MRI agents can be directly disposed on the PEG or the PEG-melanin nanoparticle surface. In other words, where the MRI agent is Gd, Gd can directly attached to the surface of the PEG-melanin nanoparticle and/or attached to the PEG via a linker compound (e.g., a chelator) such as DOTA (e.g., via a maleimide linkage (see below)). In an embodiment, all of the MRI agents are indirectly attached to the PEG-melanin nanoparticle surface via one or more linkers, such as DOTA.

An embodiment of the MRI agent can be Gd, iron oxide, paramagnetic chemical exchange saturation transfer (CEST) agents, ¹⁹F active materials, manganese, or a substance that shortens or elongates T1 or T2, and a combination thereof. The Gd MRI agent can be a compound such as DOTA-Gd, DTPA-Gd, Gd within a polymeric chelator. The iron oxide MRI agent can be a compound such as a small paramagnetic iron oxide (SPIO) or an ultrasmall SPIO with or without a dextran or other stabilizing layer. The paramagnetic CEST MRI agent can be a compound such as lanthamide complexes.

In an embodiment, the MRI agent can be linked to the PEG surface via a linkage such as a maleimide linkage, NHS ester, click chemistry, or another covalent or non-covalent approach, or a combination thereof.

In an embodiment, the PEG-melanin nanoparticle can include a radiolabel for PET or SPECT imaging. In an exemplary embodiment, the radiolabel can include one or more of the following: ⁶⁴Cu, ¹²⁴I, ^{76/77}Br, ⁸⁶Y, ⁸⁹Zr, ⁶⁸Ga, ¹⁸F, ¹¹C, ¹²⁵I, ¹²⁴I, ¹³¹I, ¹²³I, ³²Cl, ³³Cl, ³⁴Cl, ⁶⁸Ga, ⁷⁴Br, ⁷⁵Br, ⁷⁶Br, ⁷⁷Br, ⁷⁸Br, ⁸⁹Zr, ¹⁸⁶Re, ¹⁸⁸Re, ⁹⁰Y, ⁸⁶Y, ¹⁷⁷Lu, or ¹⁵³Sm. In an embodiment, the PET agent can include ¹⁸F, ⁶⁴Eu, ¹¹C, ¹³N, ¹⁵O, and the like, and the SPECT agent can include ⁹⁹Tc, ⁶⁷Ga, ¹⁹²Ir, and the like.

In an embodiment, the radiolabel can be chelated to or attached to the PEG-melanin nanoparticle, where the chelator can be bonded (e.g., directly or indirectly) to the PEG or the surface of the polymeric melanin core surface. In an embodiment, 1, 2, 3, 4, or 5 radiolabels can be present on the PEG-melanin nanoparticle. In an embodiment, the radiolabels can be chelated using a chelator such as DOTA, NOTA, TETA, EDTA, Df, and DTPA, or derivatives of each of these. In an embodiment, the chelator can be DOTA.

As described above, the MRI agent, PET agent, or the SPECT agent can be attached (e.g., directly or indirectly) to the PEG-melanin nanoparticle using a chelator. In an embodiment, the chelator can be bonded to a PEG. In an embodiment, the chelator compound can include, but is not limited to, a macrocyclic chelator, a non-cyclic chelator, and combinations thereof. The macrocyclic chelator can include, but is not limited to, 1,4,7,10-tetraazadodecane-N,N′,N″,N′″-tetraacetic acid (DOTA), 1,4,7-triazacyclononane-1,4,7-triacetic acid (NOTA), 1,4,8,11-tetraazacyclotetradecane-1,4,8,11-tetraacetic acid (TETA), diethylenetriaminepentaacetic (DTPA), 4,11-bis(carboxymethyl)-1,4,8,11-tetraazabicyclo[6.6.2]hexadecane (CB-TE2A), 1-N-(4-aminobenzyl)-3,6,10,13,16,19-hexaazabicyclo[6.6.6]eicosane-1,8-diamine (SarAr), or combinations thereof.

Additional chelators that can be used in embodiments of the present disclosure include natural chelators and synthetic chelators. In an embodiment, the natural chelators include, but are not limited to, carbohydrates (e.g., polysaccharides), organic acids with more than one coordination group, lipids, steroids, amino acids, peptides, phosphates, nucleotides, tetrapyrrols, ferrioxamines, lonophores (e.g., gramicidin, monensin, and valinomycin), and phenolics. In an embodiment, the synthetic chelators include, but are not limited to, ammonium citrate dibasic, ammonium oxalate monohydrate, ammonium tartrate dibasic, ammonium tartrate dibasic solution, pyromellitic acid, calcium citrate tribasic tetrahydrate, ethylene glycol-bis(2-aminoethylether)-N,N,N,N-tetra acetic acid, sodium glycocholate, ammonium citrate dibasic, calcium citrate tribasic tetrahydrate, magnesium citrate tribasic, potassium citrate, sodium citrate monobasic, lithium citrate tribasic, sodium citrate tribasic, citric acid, N,N-dimethyldecylamine-N-oxide, N,N-dimethyldodecylamine-N-oxide, ammonium citrate dibasic, ammonium tartrate dibasic, ethylenediaminetetraacetic acid diammonium salt, potassium D-tartrate monobasic, N,N-dimethyldecylamine-N-oxide, N,N-dimethyldodecylamine-N-oxide, ethylenediaminetetraacetic acid dipotassium salt dihydrate, sodium tartrate dibasic, ethylenediaminetetraacetic acid, ethylenediaminetetraacetic acid disodium salt dihydrate, ethylenediaminetetraacetic acid, ethylenediaminetetraacetic acid tetrasodium salt hydrate, ethylenediaminetetraacetic acid tripotassium salt, ethylene glycol-bis(2-aminoethylether)-N,N,N,N-tetra acetic acid, ethylene glycol-bis(2-aminoethylether)-N,N,N,N-tetra acetic acid, potassium oxalate, sodium oxalate, ethylene glycol-bis(2-aminoethylether)-N,N,N,N-tetra acetic acid, ethylenediaminetetraacetic acid, ethylenediaminetetraacetic acid diammonium salt, ethylenediaminetetraacetic acid dipotassium salt dihydrate, ethylenediaminetetraacetic acid disodium salt dihydrate, ethylenediaminetetraacetic acid disodium salt dihydrate, ethylenediaminetetraacetic acid, ethylenediaminetetraacetic acid tetrasodium salt hydrate, ethylenediaminetetraacetic acid tripotassium salt, ethylenediaminetetraacetic acid trisodium salt trihydrate, ethylenediaminetetraacetic acid dipotassium salt dihydrate, ethylene glycol-bis(2-aminoethylether)-N,N,N,N-tetra acetic, ethylene glycol-bis(2-aminoethylether)-N,N,N,N-tetra acetic acid, sodium glycocholate, ethylene glycol-bis(2-aminoethylether)-N,N,N,N-tetra acetic acid, 5-sulfosalicylic acid, N,N-dimethyldodecylamine-N-oxide, magnesium citrate tribasic, magnesium citrate tribasic nonahydrate, ammonium oxalate monohydrate, potassium tetraoxalate, potassium oxalate, sodium oxalate, potassium citrate, ethylenediaminetetraacetic acid dipotassium salt dihydrate, potassium D-tartrate monobasic, potassium peroxodisulfate, potassium citrate monobasic, potassium citrate tribasic, potassium oxalate monohydrate, potassium peroxodisulfate, potassium sodium tartrate, potassium sodium tartrate tetrahydrate, potassium D-tartrate monobasic, potassium tetraoxalate dihydrate, pyromellitic acid hydrate, potassium sodium tartrate, potassium sodium tartrate, ethylenediaminetetraacetic acid disodium salt dihydrate, sodium citrate monobasic, sodium bitartrate, sodium tartrate dibasic, sodium bitartrate monohydrate, sodium citrate monobasic, sodium citrate tribasic dihydrate, sodium citrate tribasic, sodium glycocholate hydrate, sodium oxalate, sodium tartrate dibasic dihydrate, sodium tartrate dibasic, 5-sulfosalicylic acid dihydrate, ammonium tartrate dibasic, sodium tartrate dibasic, potassium D-tartrate monobasic, sodium bitartrate, potassium sodium tartrate, L-(+)-tartaric acid, ethylenediaminetetraacetic acid tetrasodium salt hydrate, L-(+)-tartaric acid, calcium citrate tribasic tetrahydrate, sodium glycocholate, lithium citrate tribasic, magnesium citrate tribasic, ethylenediaminetetraacetic acid tripotassium salt, sodium citrate tribasic, and ethylenediaminetetraacetic acid trisodium salt trihydrate. In particular, the chelator compound can include, but is not limited to, EDTA (ethylenediaminetetraacetic acid), DTPA (diethylenetriaminepentaacetate), DOPA (dihydroxyphenylalanine), and derivatives of each. The agent can be incorporated into the chelate compound using methods such as, but not limited to, direct incorporation, template synthesis, and/or transmetallation, as well as methods described in the Examples.

Furthermore, the PEG-melanin nanoparticle can include another agent (e.g., a chemical or biological agent), where the agent can be disposed indirectly or directly on the PEG-melanin nanoparticle. In particular, the probe can include, but is not limited to, a drug, a therapeutic agent, a radiological agent, a chemological agent, a small molecule drug, a biological agent (e.g., peptides, proteins, antibodies, antigens, and the like) and combinations thereof, that can be used to image, detect, study, monitor, evaluate, treat, and/or screen a disease, condition, or related biological event corresponding to the target. In an embodiment, the agent is included in an effective amount to accomplish its purpose (e.g., therapeutically effective amount).

In an embodiment, the PEG-melanin nanoparticles can be made using sonication procedures. In an embodiment, melanin is dissolved in a basic aqueous solution. For example, melanin can be mixed with a base such as KOH. Once the melanin is dissolved in the aqueous solution, the pH can be adjusted (e.g., an acid such as HCl) to about 7 using sonication (e.g., for about 30 seconds to 10 min) to form melanin nanoparticles. The sonication device can include an ultrasonic cell disruption system, an ultrasonic cleaner, or similar device that achieves similar amounts. In an embodiment, the dimensions of the melanin nanoparticles can be controlled by adjusting the pH and/or sonication parameters. In an embodiment the melanin nanoparticles can be separated from the aqueous solution. After separation or without separation, the pH of the aqueous solution including the melanin nanoparticles can be adjusted to a pH of about 10 using a base such as NaCl. Subsequently, PEG precursor compounds can be added to the aqueous solution to form PEG-melanin nanoparticles. The characteristics of the PEG-melanin nanoparticles can be controlled by adjusting the pH and/or the concentration or type of PEGs. Additional details are provided in the Example.

### Methods of Use

Embodiments of this disclosure include, but are not limited to: methods of imaging a sample or a subject using the PEG-melanin nanoparticle; methods of imaging a melanin-related disease (e.g., cancer or tumor) or related biological condition (e.g., hyperpigmentation and other melanin related disease), using the PEG-melanin nanoparticle; methods of diagnosing a melanin-related disease or related biological conditions using the PEG-melanin nanoparticle; methods of monitoring the progress of a melanin-related disease or related biological conditions using the PEG-melanin nanoparticle, methods of treating a melanin-related disease or related biological conditions, and the like.

Embodiments of the present disclosure can be used to image, detect, study, monitor, evaluate, assess, treat, and/or screen, the melanin-related melanoma or related biological conditions, in particular, malignant melanoma, in vivo or in vitro using PEG-melanin nanoparticle.

In a particular embodiment, the PEG-melanin nanoparticle can be used in imaging melanin-related diseases (e.g., malignant melanoma). For example, the PEG-melanin nanoparticle is provided or administered to a subject in an amount effective to result in uptake of the PEG-melanin nanoparticle into the melanin-related disease or tissue of interest. The subject is then introduced to an appropriate imaging system (e.g., photoacoustic imaging system, PET system, MRI system, etc.) for a certain amount of time. In an embodiment, the imaging device is a photoacoustic device and detecting the PEG-melanin nanoparticle can include detecting a photoacoustic signal associated with the PEG-melanin nanoparticle. The location of the photoacoustic signal correlates to the position of the disease within the subject. The melanin related disease that takes up the PEG-melanin nanoparticle could be detected using the imaging system. The location of the detected signal from the PEG-melanin nanoparticle can be correlated with the location of the melanin related disease. In an embodiment, the dimensions of the location can be determined as well. Other labeled probes can be used in a similar manner.

In an embodiment, the steps of this method can be repeated at determined intervals so that the location and/or size of the disease can be monitored as a function of time and/or treatment. In particular, the PEG-melanin nanoparticle can find use in a host undergoing chemotherapy or other treatment (e.g., using a drug), to aid in visualizing the response of a disease or tumor to the treatment. In this embodiment, the PEG-melanin nanoparticle is typically visualized and sized prior to treatment, and periodically (e.g., daily, weekly, monthly, intervals in between these, and the like) during chemotherapy, radiotherapy, and the like, to monitor the tumor size. Other labeled probes can be used in a similar manner.

It should be noted that the amount effective to result in uptake of the PEG-melanin nanoparticle into the cells or tissue of interest may depend upon a variety of factors, including for example, the age, body weight, general health, sex, and diet of the host; the time of administration; the route of administration; the rate of excretion of the specific probe employed; the duration of the treatment; the existence of other drugs used in combination or coincidental with the specific composition employed; and like factors well known in the medical arts.

### Kits

The present disclosure also provides packaged compositions or pharmaceutical compositions comprising a pharmaceutically acceptable carrier and a PEG-melanin nanoparticle of the disclosure. In certain embodiments, the packaged compositions or pharmaceutical composition includes the reaction precursors to be used to generate the labeled probe according to the present disclosure. Other packaged compositions or pharmaceutical compositions provided by the present disclosure further include indicia including at least one of: instructions for using the labeled probe to image a host, or host samples (e.g., cells or tissues), which can be used as an indicator of conditions including, but not limited to, melanin related disease and biological related conditions.

Embodiments of this disclosure encompass kits that include, but are not limited to, the PEG-melanin nanoparticle and directions (written instructions for their use). The components listed above can be tailored to the particular biological condition to be monitored as described herein. The kit can further include appropriate buffers and reagents known in the art for administering various combinations of the components listed above to the host cell or host organism. The imaging agent and carrier may be provided in solution or in lyophilized form. When the imaging agent and carrier of the kit are in lyophilized form, the kit may optionally contain a sterile and physiologically acceptable reconstitution medium such as water, saline, buffered saline, and the like.

### Dosage Forms

Embodiments of the present disclosure can be included in one or more of the dosage forms mentioned herein. Unit dosage forms of the pharmaceutical compositions (the “composition” includes at least the labeled probe, e.g., PEG-melanin nanoparticle) of this disclosure may be suitable for oral, mucosal (e.g., nasal, sublingual, vaginal, buccal, or rectal), parenteral (e.g., intramuscular, subcutaneous, intravenous, intra-arterial, or bolus injection), topical, or transdermal administration to a patient. Examples of dosage forms include, but are not limited to: tablets; caplets; capsules, such as hard gelatin capsules and soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms of the compositions of the disclosure typically vary depending on their use. For example, a parenteral dosage form may contain smaller amounts of the active ingredient than an oral dosage form used to treat the same condition or disorder. These and other ways in which specific dosage forms encompassed by this disclosure vary from one another will be readily apparent to those skilled in the art (See, e.g., Remington's Pharmaceutical Sciences, 18th ed., Mack Publishing, Easton, Pa. (1990)).

Typical compositions and dosage forms of the compositions of the disclosure can include one or more excipients. Suitable excipients are well known to those skilled in the art of pharmacy or pharmaceutics, and non-limiting examples of suitable excipients are provided herein. Whether a particular excipient is suitable for incorporation into a composition or dosage form depends on a variety of factors well known in the art including, but not limited to, the way in which the dosage form will be administered to a patient. For example, oral dosage forms, such as tablets or capsules, may contain excipients not suited for use in parenteral dosage forms. The suitability of a particular excipient may also depend on the specific active ingredients in the dosage form. For example, the decomposition of some active ingredients can be accelerated by some excipients, such as lactose, or by exposure to water. Active ingredients that include primary or secondary amines are particularly susceptible to such accelerated decomposition.

The disclosure encompasses compositions and dosage forms of the compositions of the disclosure that can include one or more compounds that reduce the rate by which an active ingredient will decompose. Such compounds, which are referred to herein as “stabilizers,” include, but are not limited to, antioxidants such as ascorbic acid, pH buffers, or salt buffers. In addition, pharmaceutical compositions or dosage forms of the disclosure may contain one or more solubility modulators, such as sodium chloride, sodium sulfate, sodium or potassium phosphate, or organic acids. An exemplary solubility modulator is tartaric acid.

“Pharmaceutically acceptable salt” refers to those salts that retain the biological effectiveness and properties of the free bases and that are obtained by reaction with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid, malic acid, maleic acid, succinic acid, tartaric acid, citric acid, and the like.

Embodiments of the present disclosure include pharmaceutical compositions that include the labeled probe (e.g., PEG-melanin nanoparticle), pharmaceutically acceptable salts thereof, with other chemical components, such as physiologically acceptable carriers and excipients. One purpose of a pharmaceutical composition is to facilitate administration of labeled probe (e.g., PEG-melanin nanoparticle) to a subject (e.g., human).

Embodiments of the present disclosure may be salts and these salts are within the scope of the present disclosure. Reference to a compound of any of the formulas herein is understood to include reference to salts thereof, unless otherwise indicated. The term “salt(s)”, as employed herein, denotes acidic and/or basic salts formed with inorganic and/or organic acids and bases. In addition, when an embodiment of the present disclosure contains both a basic moiety and an acidic moiety, zwitterions (“inner salts”) may be formed and are included within the term “salt(s)” as used herein. Pharmaceutically acceptable (e.g., nontoxic, physiologically acceptable) salts are preferred, although other salts are also useful, e.g., in isolation or purification steps which may be employed during preparation. Salts of the compounds of an active compound may be formed, for example, by reacting an active compound with an amount of acid or base, such as an equivalent amount, in a medium such as one in which the salt precipitates or in an aqueous medium followed by lyophilization.

Embodiments of the present disclosure that contain a basic moiety may form salts with a variety of organic and inorganic acids. Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides (formed with hydrochloric acid), hydrobromides (formed with hydrogen bromide), hydroiodides, 2-hydroxyethanesulfonates, lactates, maleates (formed with maleic acid), methanesulfonates (formed with methanesulfonic acid), 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates such as tosylates, undecanoates, and the like.

Embodiments of the present disclosure that contain an acidic moiety may form salts with a variety of organic and inorganic bases. Exemplary basic salts include ammonium salts, alkali metal salts such as sodium, lithium, and potassium salts, alkaline earth metal salts such as calcium and magnesium salts, salts with organic bases (for example, organic amines) such as benzathines, dicyclohexylamines, hydrabamines (formed with N,N-bis(dehydroabietyl)ethylenediamine), N-methyl-D-glucamines, N-methyl-D-glucamides, t-butyl amines, and salts with amino acids such as arginine, lysine, and the like.

Basic nitrogen-containing groups may be quaternized with agents such as lower alkyl halides (e.g., methyl, ethyl, propyl, and butyl chlorides, bromides and iodides), dialkyl sulfates (e.g., dimethyl, diethyl, dibutyl, and diamyl sulfates), long chain halides (e.g., decyl, lauryl, myristyl and stearyl chlorides, bromides and iodides), aralkyl halides (e.g., benzyl and phenethyl bromides), and others.

Solvates of the compounds of the disclosure are also contemplated herein. Solvates of the compounds are preferably hydrates.

The amounts and a specific type of active ingredient (e.g., PEG-melanin nanoparticle) in a dosage form may differ depending on various factors. It will be understood, however, that the total daily usage of the compositions of the present disclosure will be decided by the attending physician or other attending professional within the scope of sound medical judgment. The specific effective dose level for any particular host will depend upon a variety of factors, including for example, the activity of the specific composition employed; the specific composition employed; the age, body weight, general health, sex, and diet of the host; the time of administration; the route of administration; the rate of excretion of the specific compound employed; the duration of the treatment; the existence of other drugs used in combination or coincidental with the specific composition employed; and like factors well known in the medical arts. For example, it is well within the skill of the art to start doses of the composition at levels lower than those required to achieve the desired effect and to gradually increase the dosage until the desired effect is achieved.

### EXAMPLES

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

### Example 1

### Ultrasmall Water-Soluble Melanin Nanoparticles as Platform for Multifunctional Applications

### Brief Introduction:

In this work, we report a facile method to prepare water-soluble biopolymeric melanin nanoparticles (NPs) and for the first time demonstrate their applications as exogenous polymeric nanoprobes for mulitmodel imaging in small animal models and further as platform for drug delivery. Under the careful pH-controlling, melanin and PEG-modified melanin (PEG-melanin) NPs (˜4-8 nm) can be easily prepared in high water monodispersity and homogeneity. Their optophysical stabilities were further confirmed by UV-Vis-NIR spectrometer, and their PAI ability was studied in a phantom study. Furthermore, PEG-melanin NP was radiolabeled with positron emission computed tomography (PET) radionuclide ⁶⁴Cu for understanding the in vivo biodistribution and clearance of the NPs. Lastly, PEG-melanin NPs were injected to mice (n=3 per group) either subcutaneously or through tail vein and imaged by a PAI instrument. PEG-melanin NPs did not show any noticeable cell toxicity, and ⁶⁴Cu radiolabeled melanin NPs were found to be cleared mainly through liver and some through kidney system. More importantly, melanin NPs also produce high PAI signal in vitro and in vivo. After injection with melanin NPs for 1 h, The PAI signal of blood vessel enhanced greatly and the PAI signal also appeared on the liver surface. Thus, small water-soluble biopolymeric melanin NPs can be chemically prepared and used for in vivo PAI. Combined with the multifunctions and biodegradability of melanin, such water-soluble NPs can serve as a promising platform for molecular imaging and drug delivery.

### Introduction:

Photoacoustic imaging (PAI) is a nonionizing, noninvasive emerging technique in molecular imaging that can provide strong optical absorption contrast and high ultrasonic resolution.^{[1]} In PAI, pulsed laser light beats on the body, and the ultrasound signals formed by light absorption components in various tissues are collected to construct the imaging. Such an imaging modality has been widely developed toward clinical applications. For PAI, introducing exogenous contrast agent generally adopted to greatly enhance the molecular sensitivity. Those exogenous contrast agents usually consist of highly absorbing organic dyes, such as indocyanine green (ICG)^{[2,3]}, and other NIR dyes^{[4-6]} or inorganic nanoparticles, such as gold^{[2-10]}, silver^{[11]}, copper^{[12-13]}, and carbon nanotubes^{[14-16]} that can be conjugated to a targeting moiety of interest. Despite their good properties, the intrinsic optical instability of organic dyes, and the relatively large size (>60 nm) non-biodegradability and potential biotoxicity of those inorganic nanomaterials still need to be further assessed for molecular imaging in living body.

Previously the use of endogenous contrast agent in PAI for cancer detection has made it a popular technique in recent years. In such, hemoglobin in blood which can afford the levels of vascularization and oxygen saturation in tumors is the most widely interrogated for tumor imaging^{[12]}. However, compared with exogenous contrast agent that can be easily modified for various molecular imaging, the endogenous contrast agent was only applicable on their limited position in living body.

Most recently, developing endogenous contrast agents or their analogies as exogenous contrast agents for molecular imaging is becoming a growing interest on account of their high PA signals, good biocompatibility and biodegradability. Porphyrin, one of the major components in hemoglobin, is now developed as an exogenous contrast agent for both US and PA imaging^{[18-19]}. However, few works are focused on melanin, another important endogenous contrast agent which is overexpressed in melanoma. Melanin, the well-known biopolymer which is believed to be oxidation products of tyrosine and biodegradable, plays an important role in living organism^{[20]}. Up to now, melanin was found to exhibit multifunctions in biosystem, including photoprotection, photosensitization, scavenging free radicals, chelating metal ions, binding proteins and drugs and so on^{[21-24]}. Therefore, the relationship between the optical, redox, and aggregation properties of melanin with their functions in biosystem has stimulated numerous studies^{[25, 26]}. Accompanied with the development of molecular imaging in the past decade, melanin has been used as an effective molecular target^{[27-29]} as well as endogenous contrast agent for PAI because of its strong light absorption properties.^{[30]}

Despite its important function of melanin and biodegrability, developing melanin for molecular imaging was highly subjected to its intrinsic poor water-solubility. To avoid this dilemma, our group and Wang's group recently developed melanin as report gene to realize PAI in vitro and in vivo^{[31-32]}. However, this method is too complicated, time-consuming and target-limited. Thus, developing water-soluble melanin with multifunctionalities as exogenous contrast agent will surely expand its use for molecular imaging of different diseases beyond melanoma. Up to now, only few melanin NPs which can be dispersed in water have been reported, generally isolated from the natural source, such as sepia melanin nanoparticles, or some melanin-like nanoparticles using bottom-up method through polymerization of dopamine^{[33]}. However, these nanoparticle sizes are generally larger than 100 nm and poor controllable, which are not good for molecular imaging in vivo. We herein report a facile method to prepare ultrasmall water-soluble melanin NPs through top-down method and for the first time demonstrate their applications as exogenous polymeric nanoprobes for mulitmodel imaging in small animal models and further as platform for drug delivery. Ultrasmall melanin NPs with 4.5 nm diameter were prepared in high water monodispersity and homogeneity from the commercialized water-insoluble melanin by careful pH adjusting under the assistance of sonication. After surface-modification with PEG, the melanin NPs exhibited excellent optical stability and little cell toxicity. Positron emission computed tomography (PET) was used to investigate the metabolism of the organic nanoprobe, melanin. ⁶⁴Cu radiolabeled melanin NPs were found to be cleared mainly through liver and some through kidney system. More importantly, melanin NPs also produce high PAI signal in vitro and in vivo. Further investigation showed that melanin can strongly chelate with ⁶⁴Cu and Fe³⁺ for good PET and Magnetic Resonance Imaging (MRI), and it can also binding drugs for drug delivery. Combined with the multifunctions and biodegradability of melanin, such ultrasmall water-soluble NPs with high specific surface area can serve as a promising platform for future molecular imaging and therapy.

### Materials:

The following reagents were acquired and used as received: melanin prepared by oxidation of tyrosine with hydrogen peroxide (Sigma Aldrich), sodium hydroxide (Sigma Aldrich), hydrochloric acid (37 wt %, Sigma Aldrich), NH₄OH solution (28 wt %, Sigma Aldrich) thiol-PEG₅₀₀₀-amine (SH-PEG₅₀₀₀-NH₂, SkDa, Laysan Bio), 2,2′,2″-(1,4,7-triazacyclononane-1,4,7-triyl)triacetic acid (NOTA, Macrocyclics), dimethylthiazolyl-diphenyltetrazolium (MTT; Biotium), phosphate buffered saline (PBS, Gibco), and agarose (Invitrogen). Millipore water (at 18 MOhm) was used.

### Experiments:

### Preparation of Water-Soluble Melanin (WS-Melanin) NPs.

20 mg tyrosine-derived synthetic melanin was firstly dissolved in 10 mL 0.1M KOH aqueous solution under vigorous stirring. After dissolving, 0.1M HCl aqueous solution was swiftly dropped into the obtained basic melanin solution to adjust pH=7 under sonication with output power=10 W for 1 min and a pure black melanin aqueous solution was obtained. The neutralized solution was further centrifuged with a centrifugal-filter (Amicon centrifugal filter device, MWCO=30 KDa) and washed with deionized water and repeated several times to remove the produced NaCl. The aqueous solvent was removed by freeze-drying to obtain 15 mg black solid of WS-melanin NPs.

Surface Modification of Melanin NPs with SH-PEG₅₀₀₀-NH₂ (PEG-Melanin).

NH₄OH solution (28 wt %) was added to 5 mL of melanin aqueous solution (1 mg/mL of water) to adjust the pH of the solution to 10. SH-PEG₅₀₀₀-NH₂ (5 mg, 10 mg, 25 mg, and 50 mg) was added to this mixed solution. After vigorous stirring for 12 h, surface-modified melanin nanoparticles were retrieved by centrifugation with a centrifugal-filter (Amicon centrifugal filter device, MWCO=30 KDa) and washed with deionized water several times by redispersion/centrifugation processes to remove the unreacted SH-PEG₅₀₀₀-NH₂. The aqueous solvent was removed by freeze-drying and the PEG-modified melanin was weighed to calculate the quantity of the PEG attached on melanin.

Conjugation of Melanin NPs with cRGD (RGD-Melanin):

The crosslinker solution was prepared freshly. The sulfo-SMCC (1.2 mg) was first dissolved in 36 μL of DMSO. The water-soluble PEG-melanin NPs (1.0 mg) were in 10 mM PBS (pH=7.2) were incubated with the above crosslinker solution for 2 hours at room temperature. The resultant thiol-active melanin NPs ran through a PD-10 column pre-washed with 10 mM PBS (pH=7.2) to remove excessive sulfo-SMCC and by-products. The purified melanin NPs were concentrated to the final volume of 0.5 mL with a centrifugal-filter. The cRGDfC stock solution (120 μL of 5 mM in the degassed water, 0.25 μmol) was added to the above NP solution with stirring. The conjugation reaction proceeded for 24 h at 4° C. The uncoupled RGD and byproducts were removed through PD-10 column. The resultant product, RGD-melanin, was concentrated by a centrifugal-filter and stored at 4° C. for one month without losing targeting activity. The final RGD-melanin was reconstituted in PBS and filtered through a 0.22 μm filter for cell and animal experiments.

### Characterization of Melanin NPs:

FT-IR spectra were measured in a transmission mode on a Bio-Rad FT-IR spectrophotometer (Model FTS135) under ambient conditions. Samples of pristine and functionalized melanin NPs were ground with KBr and then compressed into pellets. Transmission electron microscopy (TEM) images were recorded on a JEOL 2010 transmission electron microscope at an accelerating voltage of 100 kV. The TEM specimens were made by placing a drop of the nanoparticle aqueous solution on a carbon-coated copper grid. The hydrodynamic sizes of the melanin NPs were determined by dynamic light scattering (DLS) using a 90 Plus particle size analyzer (Malvern, Zetasizer Nano ZS90). Zeta potentials were measured using a zeta potential analyzer (Malvern, Zetasizer Nano ZS90). The ¹H-NMR spectra were recorded at 20° C. on a 400 MHz NMR spectrometer (Bruker), using D₂O as solvent. The molecular weight of the melanin NPs was measured on Shim-pack GPC-80X columns with water as the eluent and polyethyleneglycols as standard.

Conjugation of Melanin NPs with NOTA (NOTA-PEG-Melanin).

The NOTA-NHS-ester (2.3 mg) was first dissolved in 50 μL of 10 mM phosphate buffered saline (PBS, pH=7.2). 1 mg of PEG-melanin was dissolved in 1 mL of 10 mM PBS (pH=7.2). Then the PEG-melanin was incubated with the above NOTA-NHS solution for 2 h at room temperature. The result NOTA-NHS-melanin was then purified through a PD-10 column (GE, Healthcare, Piscataway, N.J.) pre-washed with 10 mM PBS (pH=7.2) to remove excessive NOTA-NHS and by-products. The purified NOTA-PEG-melanin was concentrated to the final volume of 0.3 mL.

### ⁶⁴Cu Radiolabeling.

The NOTA-PEG-melanin or RGD-melanin as radiolabeled with ⁶⁴Cu by addition of 1.13 mCi of ⁶⁴CuCl₂ in 0.1 N NaOAc (pH5.5) buffer followed by a 1 h incubation at 40° C. The radiolabeled complex was then purified by a PD-10 column (GE Healthcare, Piscataway, N.J., USA). The product was washed out by PBS and passed through a 0.22-μm Millipore filter into a sterile vial for in vitro and animal experiments.

### Fe³⁺ Labeling.

The RGD-melanin (1 mg in 1 mL H₂O) was labeled with Fe³⁺ by addition of 20 μL of FeCl₃ (10 mg/mL) in PBS (pH=7.4) followed by a 1 h incubation at 40° C. The labeled complex was then purified by a PD-10 column. The product was washed out by PBS and passed through a 0.22-μm Millipore filter into a sterile vial for in vitro and animal experiments.

### Drug Delivery and Therapy Efficiency:

0.5 mg 3,3′-methylenediindole (MDI) was dissolved in THF and then incubated with 1 mg melanin in aqueous solution for 2 h and finally the THF solvent was volatilized naturally. The study of drug loading amount was conducted through UV absorption detection. UV absorption intensity at 300 nm belonging to MDI was used to calculate drug loading amount of MDI to melanin. The MDI-melanins with different concentrations were further incubated with U87-MG cells to test their therapy efficiency in vitro.

### Cell Culture:

In vitro cytotoxicity of melanin and M-PEG-NH₂ was determined in NIH-3T3 cells culture system by the MTT assay which is on the basis of the ability of the mitochondrial succinate-tetrazolium reductase system to convert 3-(4,5-dimethylthiazol-2-yl)-2,5-diphenyltetrazolium bromide (MTT) to a purple-colored formazan in living cells. NIH-3T3 cells were incubated on 96-well plate in DMEM medium containing 10% FBS and 1% penicillin/streptomycin at 37° C. in 5% CO₂ humidified atmosphere for 24 h and 0.5×10⁴ cells were seeded per well. Cells were then cultured in the medium supplemented with indicated doses of MS-melanin or PEG-melanin for 24 h. The final concentration of PFNBr in the culture medium was fixed at 0.002, 0.02, and 0.2 mg/mL in the experiment. Addition of 10 μL of MTT (0.5 mg/mL) solution to each well and incubation for 3 h at 37° C. was followed to produce formazan crystals. Then, the supernatant was removed and the products were lysed with 200 μL of dimethylsulfoxide (DMSO). The absorbance value was recorded at 590 nm using a microplate reader. The absorbance of the untreated cells was used as a control and its absorbance was as the reference value for calculating 100% cellular viability.

### PAI Analysis of Phatom:

For the PA signal studies of melanin NPs, a cuboid container was half filled with 1% agarose gel to half depth. Different concentrations of melanin NP aqueous solutions ranging from 0.05 mg/mL to 1.6 mg/mL were filled into polyethylene capillaries and then the capillaries were laid on the surface of solidified agarose gel. The capillaries were further covered with thin 1% agarose gel to make the surface smooth. For the particle's sensitivity in living body, melanin NPs aqueous solution with different concentrations from 0.4 mg/mL to 6.4 mg/mL were mixed with matrigel at 0° C. and then subcutaneous injected on the lower back of mice. The PAIs of the mixtures were collected after they were solidified.

The Vevo LAZR PAI System (VisualSonics Inc., Toronto, Canada) with a laser at excitation wavelength of 680 nm and a focal depth of 10 mm was used to acquire photoacoustic and ultrasound images. Image analysis was carried out using ImageJ. Briefly, quantification analysis was performed on the PAI images. All slices of a sample were stacked by Z-Project with the maximum intensity, and ROIs were drawn over the cell sample on the stacked PAI images. The PAI signal intensity was then measured using the ROIs manager tool.

### In Vivo PAI Analysis of Mice:

All animal experiments were performed in compliance with the Guidelines for the Care and Use of Research Animals established by the Stanford University Animal Studies Committee. Mice were anesthetized with 2% isoflurane in oxygen and placed with lateral position. PAI was carried out using the same Vevo LAZR PAI System as the in vitro study. Similarly, image analysis was carried out using ImageJ, and quantification analysis was performed on the PAI.

### Biodistribution Studies:

For biodistribution studies, mice (n=3) were sacrificed at 24 h after tail vein injection of 114 μCi of 64Cu-NOTA-PEG-melanin and RGD-melanin NPs. Normal tissues of interest were removed and weighed, and radioactivity was measured by gamma-counter. The radioactivity uptake in the tumor and normal tissues was expressed as a percentage of the injected radioactive dose per gram of tissue (% ID/g).

### Subcutaneous Tumor Models:

All animal studies were carried out in compliance with federal and local institutional rules and approved by the Stanford University Animal Care and Use Committee (IACUC). Female athymic nude mice (nu/nu) in 4-6 weeks old were obtained from the Charles River Laboratories (Boston, Mass., USA) and kept under sterile conditions. U87-MG cells suspended in 100 mL of PBS were inoculated subcutaneously in the shoulder of nude mice. When the tumors reached 0.5-0.8 cm in diameter, the tumor bearing mice were subjected to in vivo multimodality imaging (PAI, MRI and PET) and biodistribution studies.

### PAI and MRI of Tumor Bearing Mice:

Mice bearing tumor (U87-MG) were anesthetized with 2% isoflurane in oxygen and placed with lateral position. MRI was performed using the same instrument, protocols and conditions as in the phantom MRI study. Imaging analysis was performed using the OsiriX software. The contrast was adjusted and ROIs were drawn over the tumor and muscle. T1 value of ROIs was then measured, and the ratio of tumor/muscle was calculated. PAI was carried out using the same Vevo LAZR PAI System as the in vitro study. Similarly, image analysis was carried out using ImageJ, and quantification analysis was performed on the PAI images.

### Small-Animal PET:

Small animal PET imaging of tumor-bearing mice was performed on a micro-PET R4 rodent scanner (Siemens Medical Solutions Inc., Knoxville, Tenn., USA). Mice bearing U87-MG tumors were injected with ⁶⁴Cu-melanin (180.0±5.0 μCi) via their tail vein. At different times after injection (2, 4, 12 and 24 h), the mice were anesthetized with 2% isoflurane and placed prone near the center of the FOV of the scanner. Three-minute static scans were obtained. All the small animal PET images were reconstructed by a two dimensional ordered-subsets expectation maximization (OSEM) algorithm. No background correction was performed.

### Results and Discussion:

### Preparation:

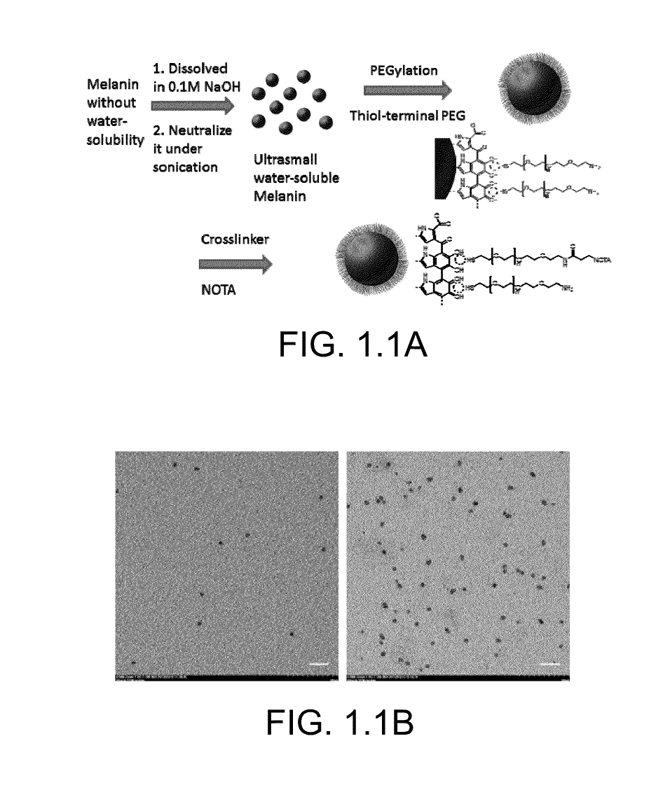

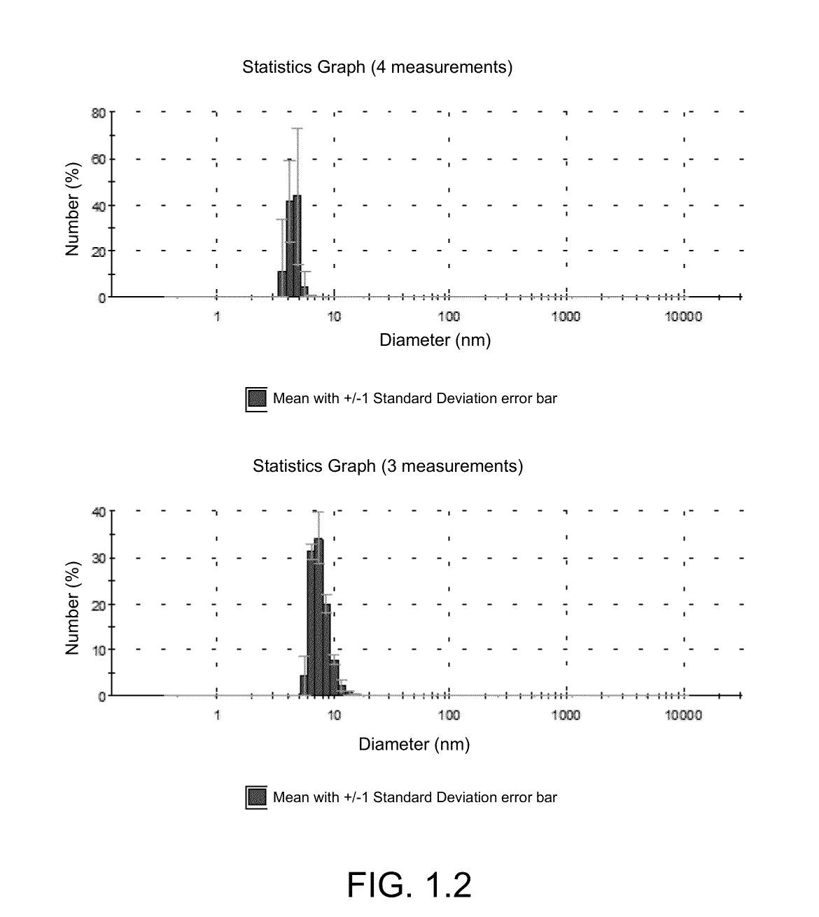

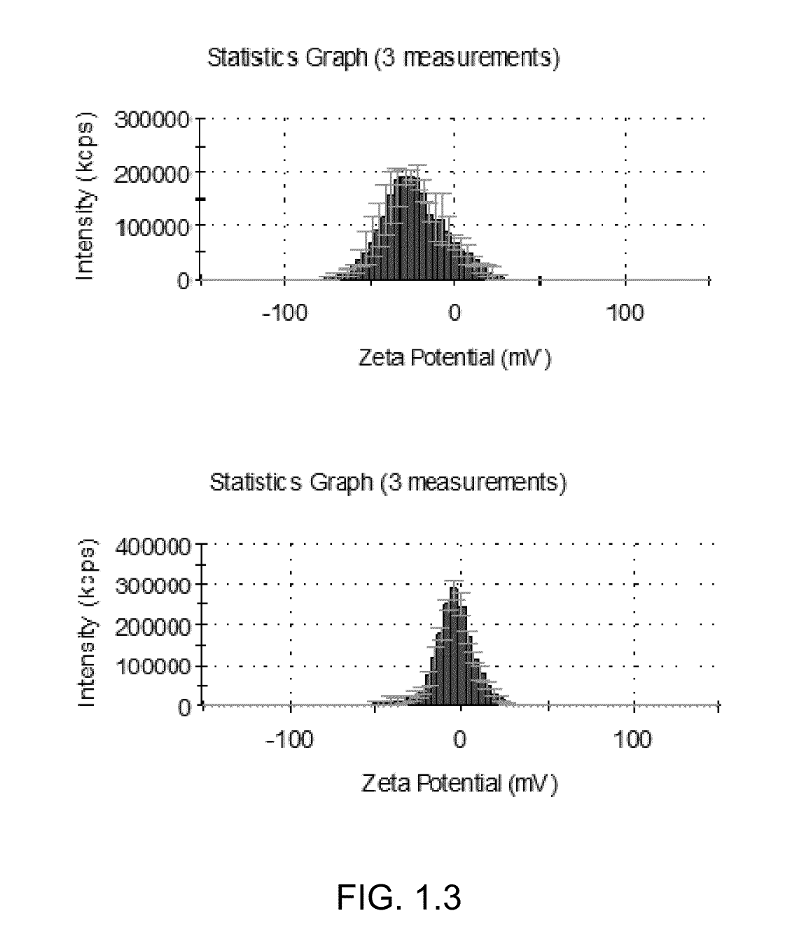

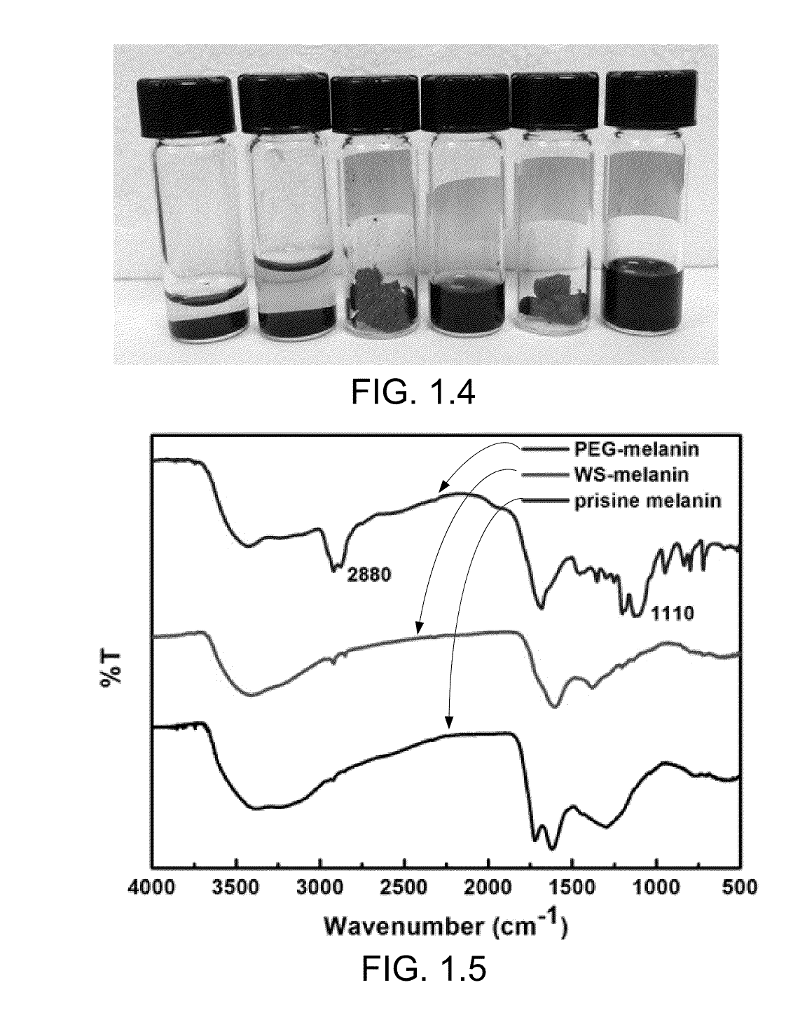

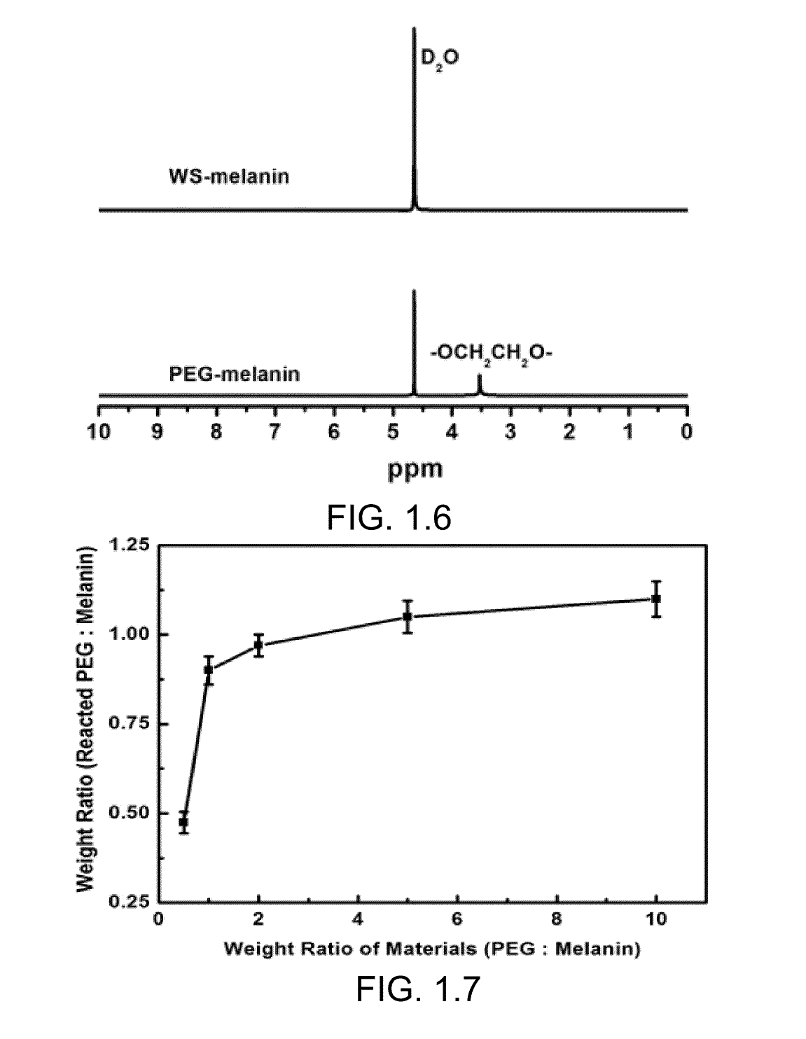

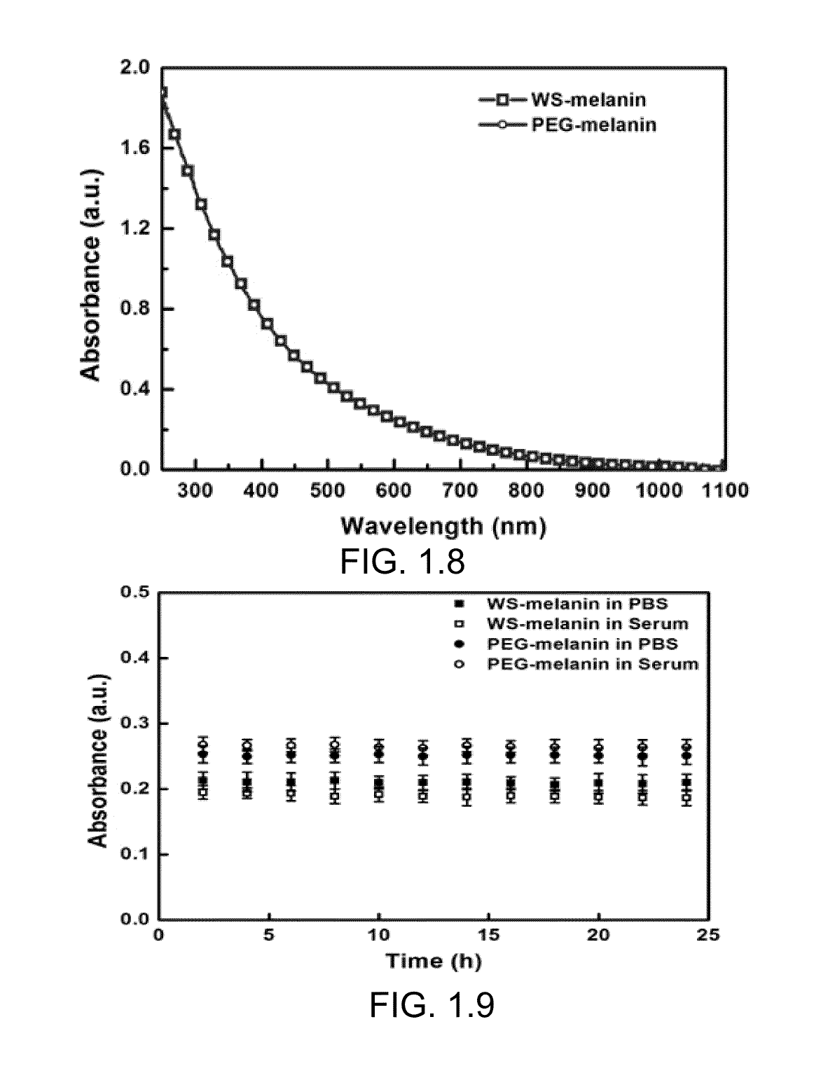

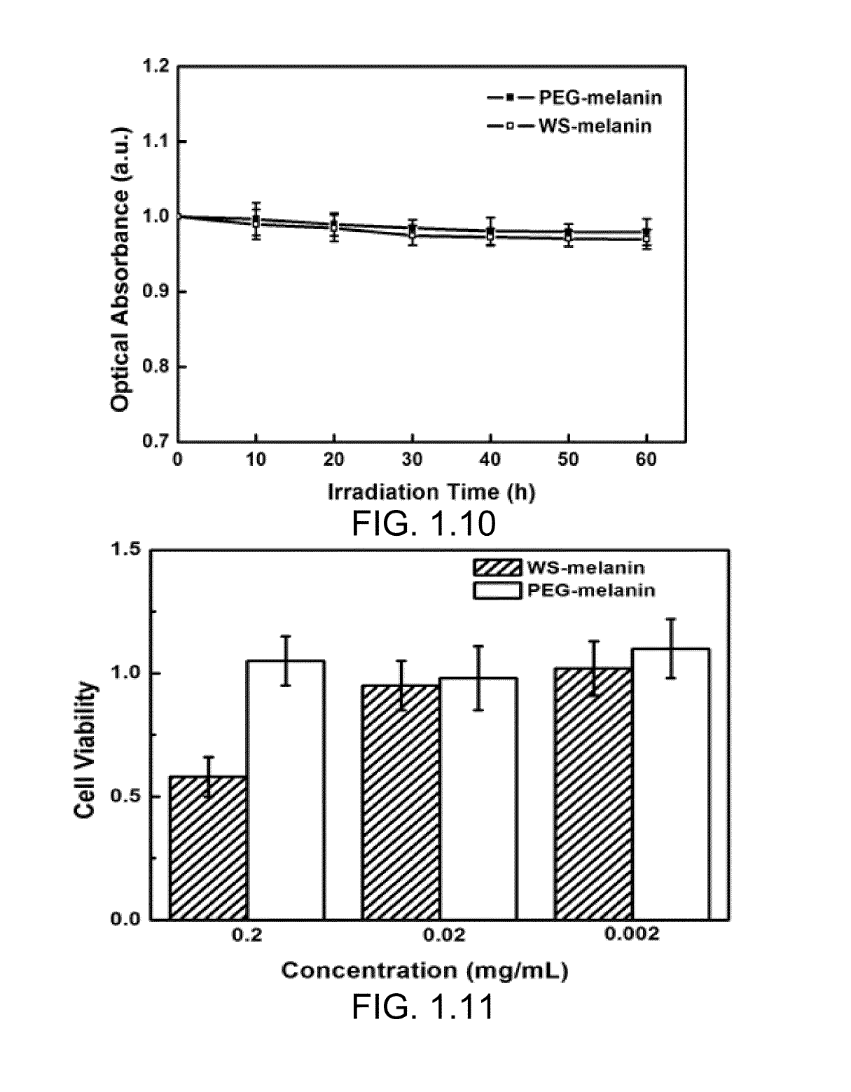

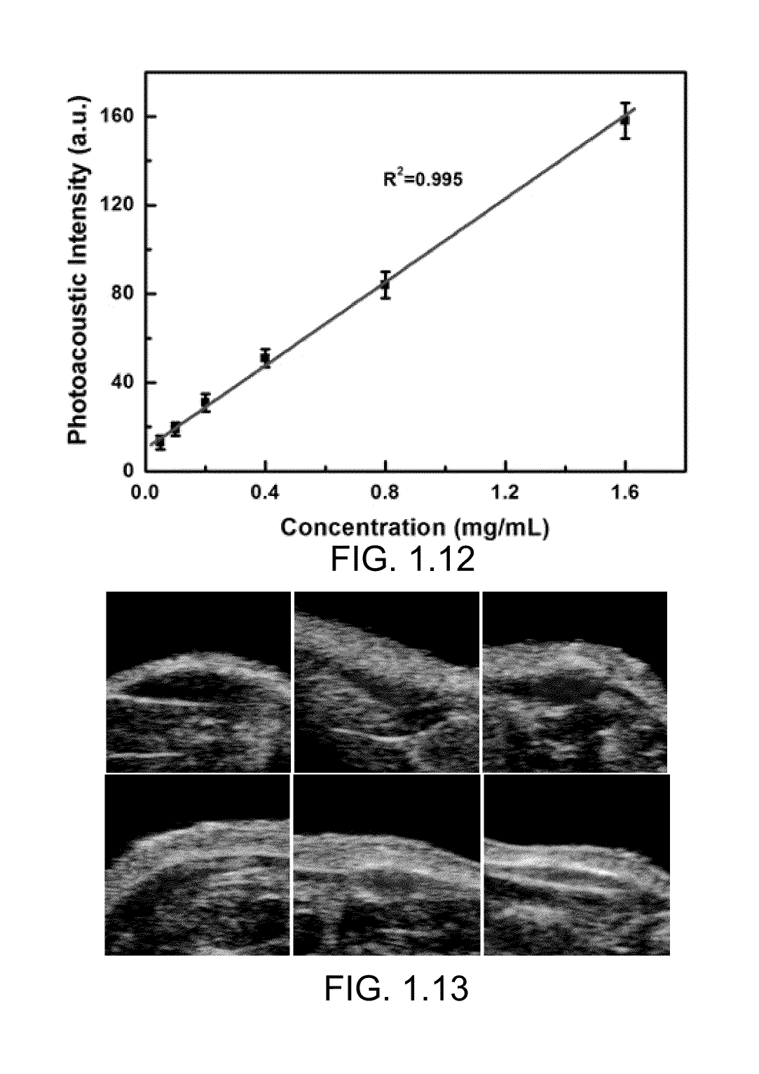

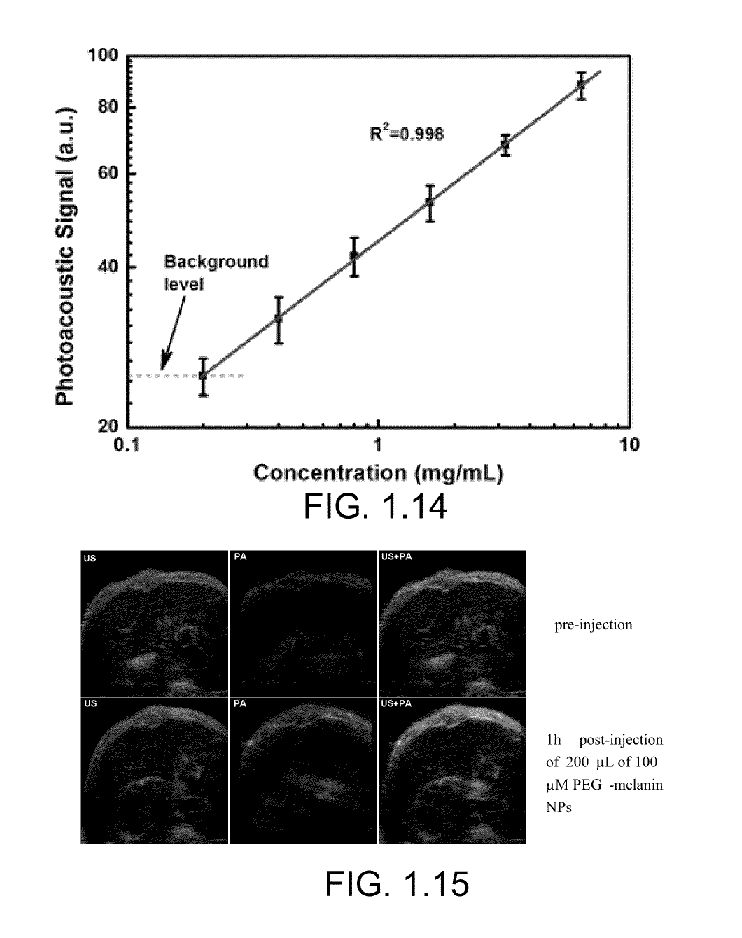

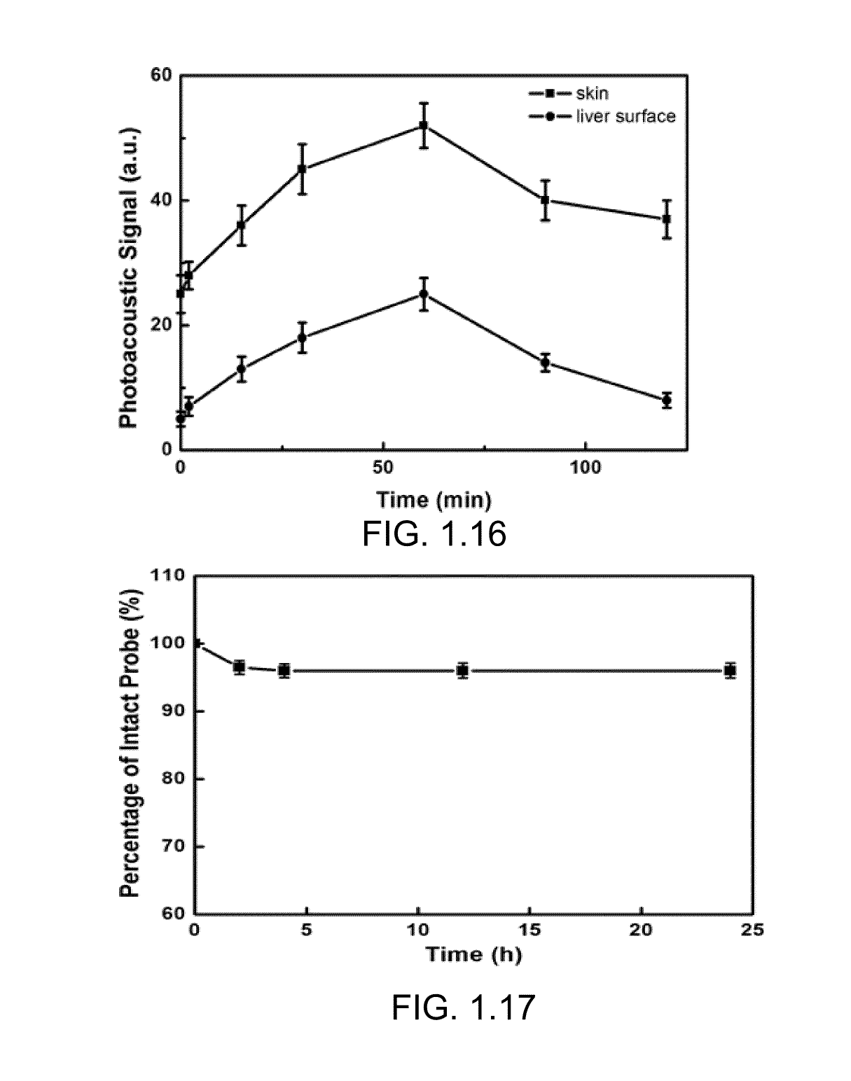

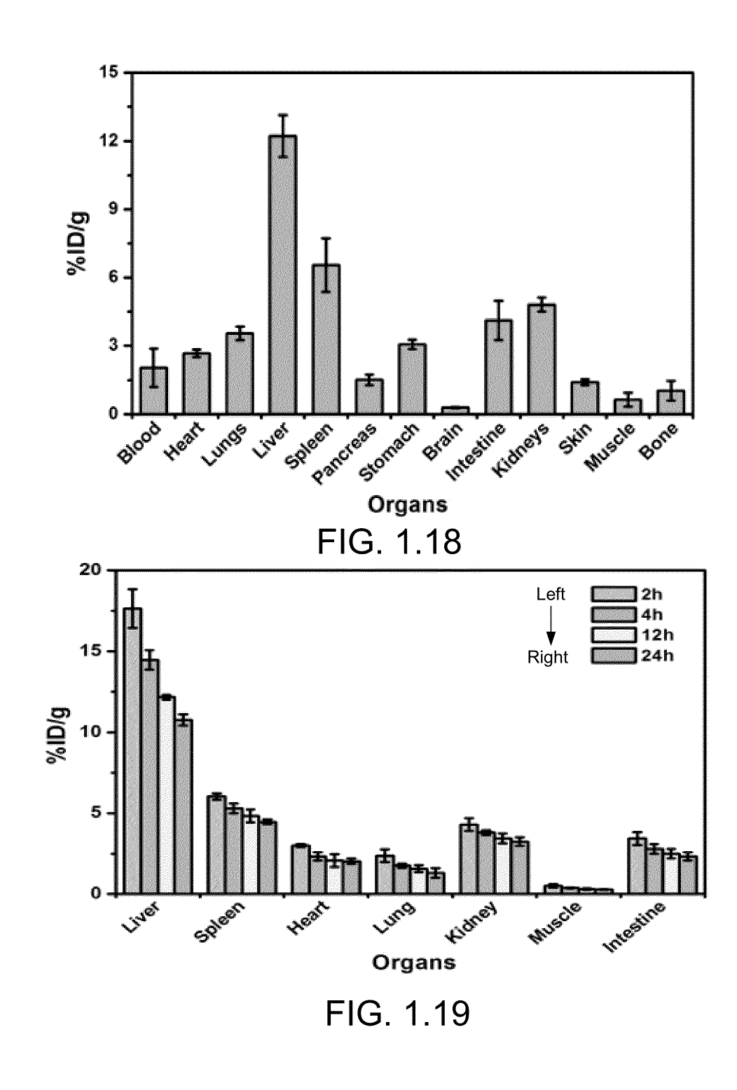

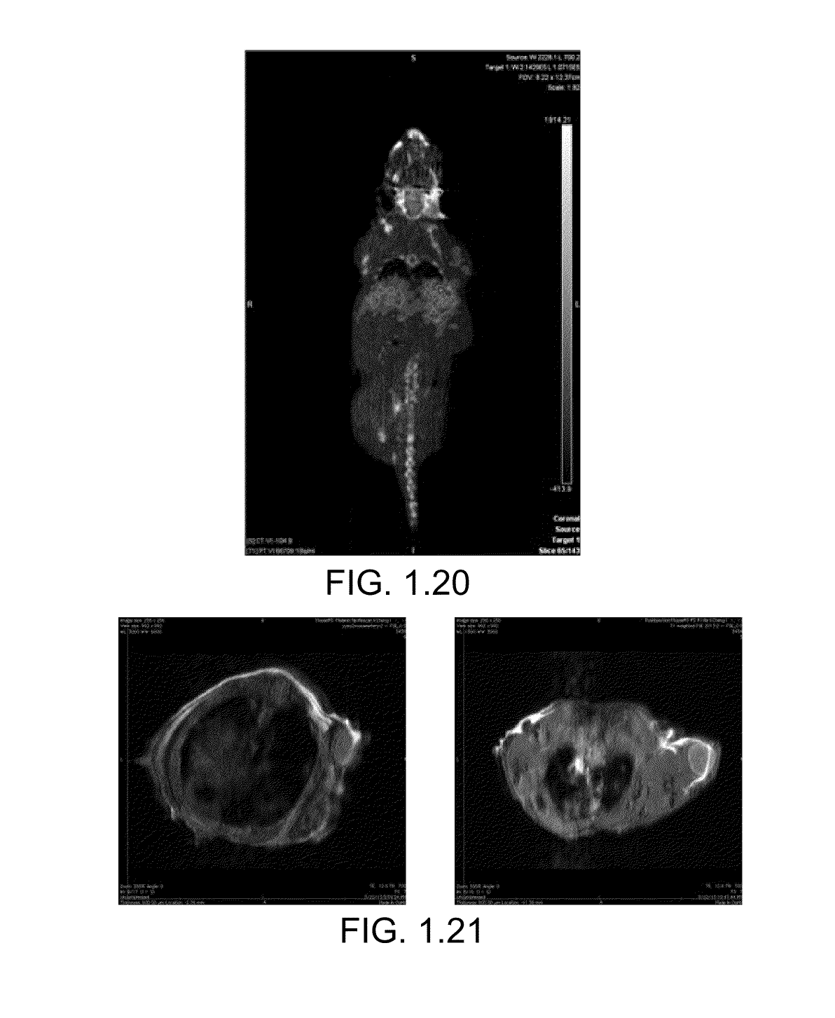

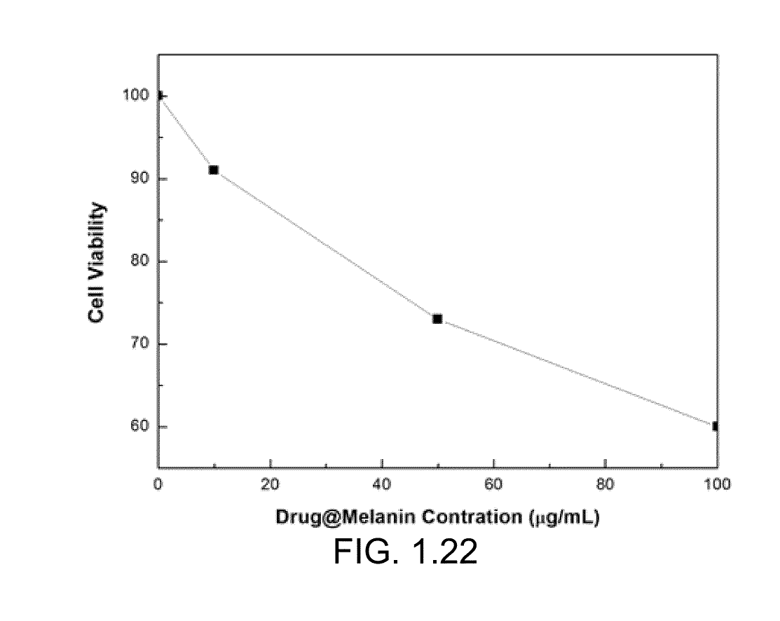

Previously it was reported that tyrosine-derived melanin can be dissolved in strong basic solution^{[34]}. Although the formation mechanism of polymeric melanin is not clear, its molecular structure is generally considered to be composed of dihydroxyindole/indolequinone segments with hydrophobic conjugated main chain and hydrophilic phenol groups on the benzene rings. It is reasonable that the phenol groups on melanin will lose their hydrogen atom to form anionic groups at a strong basic environment, and thus the formed melanin NPs with polyanions encapsulating the hydrophobic conjugated backbones can be dissolved in water. However, when neutralized, the anionic groups change back to phenol groups, leading to the decreased water-solubility and concomitantly the spontaneously increased interchain aggregation through hydrophobic and π-π interaction, and finally the rapidly formed precipitation. Thus, to keep melanin water-soluble at neutral environments, decreasing the aggregation of conjugated main chain and lowering the formed melanin particle size to expose more hydrophilic phenol groups on the surface of melanin is a promising way. Based the above considerations, we firstly dissolved pristine melanin in strong basic aqueous solution (0.1 N) and then neutralized it with the assistance of sonication to decrease the interchain aggregation (FIG. 1.1A, Scheme 1). Finally, we successfully obtained ultrasmall melanin NPs in high water monodispersity and homogeneity with the NP size of 4.5 nm (FIG. 1.1B and 1.2). The melanin exhibits excellent water-solubility of 40 mg per milliliter which can be attributed to the highly negative potential of −22.5 mV on the NP surface (FIG. 1.3) that can efficiently block the NP aggregation by electrostatic repulsion.

Compared with the rapid sinking of melanin when neutralized only under vigorous stirring, the sonication can efficiently decrease the interchain aggregation and the formed ultrasmall melanin NPs can be well-stabilized in water by the phenol groups on NP surface. Furthermore, the obtained melanin can be stored as freeze-dried solid over six months and redissolved well in water (FIG. 1.4). In FIG. 1.5, the FT-IR spectra of pristine melanin and water-soluble melanin (WS-melanin) are similar with each other, indicating no significant change of molecular structure. The ¹H NMR spectrum of WS-melanin in D₂O showed that no obvious signal which belongs to the hydrogen atom on the arylene groups was observed, indicating the conjugated backbone was well buried in the nanoparticles and cannot be touched by water solvent. Although we cannot obtain the molecular weight of pristine melanin, the molecular weight of water-soluble melanin NPs can be measured from DLS which is about 50,000 for every melanin nanoparticle.

To further enhance the biocompatibility of melanin, PEG chains were introduced to the melanin NPs. SH-PEG₅₀₀₀-NH₂ was used to react with the surface of melanin NPs because it was reported to exist reaction between terminal thiol groups and the catechol/quinine groups of polydopamine, a polymer having similar molecular structure with biopolymer melanin^{[35]}. In FT-IR spectra (FIG. 1.5), PEG-modified melanin NPs showed characteristic absorption peaks of PEG at 2880 cm⁻¹ (alkyl C—H stretching) and 1110 cm⁻¹ (C—O—C stretching). ¹H NMR further confirmed the existence of PEG on the melanin NPs. A new peak at 3.5 ppm attributing to PEG (—OCH₂CH₂O—) appeared in the ¹H NMR of PEG-melanin NPs. (FIG. 1.6) The saturation quantity of PEG reacted with melanin were also investigated. In FIG. 1.7, it was showed that the saturation weight ratio of PEG to melanin is about 1.2:1. Combined with the molecular weight of melanin particles, the number of PEG chain on every melanin particle is about 12. In TEM, after adding PEG, the diameter of PEG-melanin NP became large which is about 7 nm. Also, the surface potential of PEG-melanin NP decreased from −22.5 mV to −4.5 mV (FIG. 1.3), due to the introduction of PEG and the positive NH₂ group on the melanin NP surface. The similar absorption spectrum of PEG-melanin NPs with the water-soluble melanin NP demonstrates the PEG-modification little influences the absorption properties of melanin (FIG. 1.8).

### Nanoparticle Stability:

The stability of the melanin NPs in serum and PBS was investigated. We incubated 0.2 mg/mL water-soluble melanin and PEG-melanin NPs with 1 mL 10% mouse serum and 90% PBS 1× at 37° C. and monitored the optical absorbance of the solution at 680 nm respectively every 1 h for a period of 24 h. Control solutions included 10% serum in PBS only, and water-soluble melanin or PEG-melanin NPs in PBS without serum. The optical absorbance remained steady during the 24 h incubation (standard deviation of the absorbance was 2% and 1% of the average absorbance and the maximum deviation from average was below 3% and 2% for melanin and PEG-melanin NPs, respectively). (FIG. 1.9) The optical stability of water-soluble melanin and PEG-melanin NPs under increasing durations of light exposure (photobleaching) were further tested. Compared with those reported dyes for PA application which exhibit reduced absorption higher than 30% under light exposure, all melanin NPs were found to be intriguing photo-stable (only 3% reduced absorption) after 60 min of continuous laser irradiation at 680 nm and 8 mJ/cm² (the maximal skin exposure used in the experiments described here). This result indicates that the obtained melanin NPs is an optical stable contrast reagent for PA imaging. (FIG. 1.10)

### Cell Viability:

Melanin is generally used for photoprotection of cells and considered containing good cell viability. However, our investigation showed that the water-soluble melanin NP exhibits cytotoxicity at high concentration. After incubation of melanin with NIH3T3 cell for 24 h, nearly 50% cell was dead. (FIG. 1.11) Such increased cytotoxicity of ultrasmall melanin nanoparticles may result from their enhanced specific surface area with high charge density which may influence the cell viability. When the water-soluble melanin NPs was modified with PEG, the cell viability enhanced greatly. It was showed that when incubated with PEG-M at the same high concentration, the cell viability is 100%. It is noteworthy that after surface-modification with PEG, the decreased z-potential of melanin NP from high negative level (−22.5 mV) to closer neutral (−4.5 mV) level as well as the good cell viability of PEG chains may help to explain the increased cell viability of melanin NP. Based on the above results, our next work on imaging in vivo focused on PEG-melanin.

### Photoacoustic Imaging of Melanin Nanoparticles:

We constructed a nonabsorbing and nonscattering agarose phantom with inclusions of PEG-melanin NPs at increasing concentrations from 0.05 mg/mL to 1.6 mg/mL in multiples of 2 (n=3 for each concentration). All the photoacoustic signals produced by the PEG-melanin NPs increased linearly with the increase of NP concentration (R²=0.995) (FIG. 1.12).

We then tested the particle's sensitivity in living body by subcutaneous injection of PEG-melanin NPs on the lower back of mice (n=3) with 30 μL of PEG-melanin NPs mixed with matrigel at increasing concentrations of 0.4 mg/mL to 6.4 mg/mL in multiples of 2. Matrigel itself appears no obvious photoacoustic signal. After injection, the matrigel rapidly solidified at the body temperature to fix the melanin NPs in place and ultrasound and photoacoustic images were applied to study the inclusions (FIG. 1.13). Combined with the ultrasound image which can afford the visualized information of the living body, the photoacoustic image can reveal not only the enhanced contrast but also the accurate position of the contrast agent in living body. The photoacoustic signal from each inclusion was calculated using a region of interest (ROI) drawn over the whole inclusion region. A linear correlation (R²=0.998) between the melanin NP concentration and the corresponding photoacoustic signal can be observed in FIG. 1.14. The background signal from tissue was quantified using the signals from the areas without containing any contrast agent. In the signal-concentration graph, it can be extrapolated that 0.2 mg/mL of PEG-melanin NPs give the equivalent PA signal strength as the tissue background.

We then injected one group of mice (n=3) through the tail-vein with 200 μl of PEG-melanin NPs at a concentration of 5 mg/mL. Three-dimensional ultrasound and photoacoustic images of the liver and its surroundings were acquired before and up to 2 h after injection. A weak photoacoustic signal in the skin, produced by the abundant capillary blood vessel, was seen in the pre-injection (FIG. 1.15). After injection, it can be clearly observed that the PA signal in the skin rapidly strengthened due to the injected contrast agent of melanin NPs circled around the whole blood vessels in the mice. Furthermore, the strong photoacoustic signal appeared on the surface of liver. It may also be explained by the melanin NPs in the blood vessel around the liver surface through blood circulation. We calculated the photoacoustic signal by drawing ROI around the liver. The photoacoustic signal intensity of skin and liver was quantified as a function of time (FIG. 1.16). The decreased photoacoustic signal observed for the skin after 2 h post-injection is caused by the clearance of melanin NPs from the bloodstream. The PA signal on the liver surface synchronously changed with the signal in the skin, indicating the PA signal on the liver surface is highly related to the blood vessel. Although the biodistribution research below showed that most of the melanin NPs accumulated in the liver and finally cleared by liver, the PA signal on the liver surface is weaker than in the skin and the signal in the liver is hardly observable. This result shows that the PA signal strength is highly related to the environment those contrast agents stayed[36].

Biodistribution of Melanin NP with PET Imaging:

To investigation the biodistribution of melanin NPs, we selected ⁶⁴Cu as a PET radiolabel for melanin because it can be readily produced using a medical cyclotron and the intermediate half-life of ⁶⁴Cu makes it suitable for radiolabeling organic materials (small molecule, peptide and so on) [2830]. Simple reaction of PEG-melanin NP with NOTA allowed labeling melanin with ⁶⁴Cu. ⁶⁴Cu-NOTA-PEG-melanin displayed good stability in mouse serum (FIG. 1.17). The percentage of intact probe was 96.5%, 96.0%, 96.0%, and 96.0% at 2 h, 4 h, 12 h and 24 h of incubation, respectively. Decopper was not observed for ⁶⁴Cu radiolabeled Melanin incubated with mouse serum up to 24 h. Thus, ⁶⁴Cu-NOTA-PEG-melanin can be reliably produced and demonstrates good stability in vitro. Overall labeling and biodistribution studies with ⁶⁴Cu radiolabeled Melanin indicated that most of the melanin was cleared from liver and a small part of them from kidney. After 24 h, compared with other organ uptakes which are generally about 2-3% ID/g, the normal-liver, spleen and kidney uptakes are relatively high, which is 12.3, 8.3 and 5.1% ID/g respectively (FIG. 1.18). Such a metabolism process can be explained by the small size of PEG-melanin with around 8 nm which is close to the maximum NP size (˜8 nm) that can be renal filtrated and excreted by urinary system. It is well-known that the size and charge of most inorganic nanoparticles preclude their efficient clearance from the body as intact nanoparticles and thus toxicity is potentially amplified without such clearance or their biodegradation into biologically benign components. As a result, we further studied the liver-uptakes with the time change. It is intriguing to find that melanin can be rapidly and efficiently cleared by liver. In FIG. 1.19, it can be seen that at 2 h, the uptake of melanin in liver reached the highest at about 18% ID/g. After 24 h, the uptake of melanin decreased to 10.3% ID/g, only left 57% quantity in liver compared with that at 2 h. All these showed that melanin NPs can be rapidly cleared from the body. Furthermore, it has been proved that the biopolymer melanin can be slowly biodegraded and finally cleared from living body. Thus, compared with the uncertain toxicity of the residual inorganic NPs in living body, the possibly uncleaned melanin NP left in living body is anticipated to be biodegraded in the living body and the toxicity will be decreased to the minimum.

### PET of Subcutaneous Tumor:

Decay-corrected coronal (top row) small-animal PET images of U87-MG tumor-bearing mice at 24 h after tail vein injection of ⁶⁴Cu-RGD-melanin are shown in FIG. 20. U87-MG tumors were clearly visualized with good tumor-to-background contrast, indicating the successful targeting of tumor by RGD. Liver and kidney uptakes were also observed in all animals, which was consistent with our previous biodistributing results

### MRI of Subcutaneous Tumor:

To demonstrate the use of Fe³⁺-RGD-melanin as a probe for MRI of tumors, T1-weighted images were obtained from the group of mice bearing U87-MG tumor (n=4). U87-MG tumor displayed significantly high signals (FIG. 21), indicating Fe³⁺-melanin can be successfully used as a MRI probe in vivo while achieving excellent tumor contrast.

### Drug Delivery and Therapy Efficiency:

The effect of drug@melanin (MDI-melanin) with different concentrations was examined by MTT using U87-MG cells over 24 h at 37° C. drug@melanin with 100 μg/mL showed the highest toxicity. 40% cancer cell was killed after 24 h incubation with 100 μg/mL drug@melanin (FIG. 22), implying good therapeutic effect of drug@melanin. These results indicate melanin may be a good carrier for drug delivery.

### Conclusions:

In summary, we reported a facile method to prepare water-soluble melanin NPs and for the first time demonstrate their applications as exogenous polymeric nanoprobes for mulitmodel imaging in small animal models and further as platform for drug delivery. Melanin and PEG-Melanin NPs (˜4-8 nm) can be easily prepared in high water monodispersity and homogeneity. PEG-Melanin NPs did not show any noticeable cell toxicity, and ⁶⁴Cu-NOTA-PEG-melanin NPs were found to be cleared mainly through liver and some through kidney system. More importantly, melanin NPs also produce high PAI signal in vitro and in vivo. After injection with melanin NPs for 1 h, The PAI signal of blood vessel enhanced greatly and the PAI signal also appeared on the liver surface. Further investigation showed that melanin can strongly chelate with ⁶⁴Cu and Fe³⁺ for good PET and MRI, and it can also binding drugs for drug delivery. Combined with the multifunctions of melanin that can be efficiently combined with metal ions, binding drugs and so on, such ultrasmall melanin NP with good water-solubility, high specific surface area and biodegradability can serve as a promising platform for molecular imaging and therapy.

### REFERENCES

1. Lihong V. Wang and Song Hu, Photoacoustic Tomography: In Vivo Imaging from Organelles to Organs, Science 2012, 335, 1458-1462
2. Wang, X.; Ku, G.; Wegiel, M. A.; Bornhop, D. J.; Stoica, G.; Wang, L. V. Opt. Lett. 2004, 29, 730.
3. Kim, C.; Song, K. H.; Gao, F.; Wang, L. V. Sentinel Lymph Nodes and Lymphatic Vessels: Noninvasive Dual-Modality in Vivo Mapping by Using Indocyanine Green in RatsOVolumetric Spectroscopic Photoacoustic Imaging and Planar Fluorescence Imaging. Radiology 2010, 255, 442-450.
4. Walter J. Akers, Chulhong Kim, Mikhail Berezin, Kevin Guo, Ralph Fuhrhop, Gregory M. Lanza, Georg M. Fischer, Ewald Daltrozzo, Andreas Zumbusch, Xin Cai, Lihong V. Wang, and Samuel Achilefu, Noninvasive Photoacoustic and Fluorescence Sentinel Lymph Node Identification using Dye-Loaded Perfluorocarbon Nanoparticles, ACS Nano, 2011, 1, 173-182
5. Adam de la Zerda, Sunil Bodapati, Robert Teed, Salomon Y. May, Scott M. Tabakman, Zhuang Liu, Butrus T. Khuri-Yakub, Xiaoyuan Chen, Hongjie Dai, and Sanjiv S. Gambhir, Family of Enhanced Photoacoustic Imaging Agents for High-Sensitivity and Multiplexing Studies in Living Mice, ACS Nano, 2012, 6, 4694-4701.
6. Jelena Levi, Sri Rajasekhar Kothapalli, Te-Jen Ma, Keith Hartman, Butrus T. Khuri-Yakub, and Sanjiv Sam Gambhir, Design, Synthesis, and Imaging of an Activatable Photoacoustic Probe, J. AM. CHEM. SOC. 2010, 132, 11264-11269.
7. Kwang Hyun Song, Chulhong Kim, Claire M. Cobley, Younan Xia, and Lihong V. Wang, Near-Infrared Gold Nanocages as a New Class of Tracers for Photoacoustic Sentinel Lymph Node Mapping on a Rat Model, Nano Lett. 2009, 9, 183-188.
8. Dipanjan Pan, Manojit Pramanik, Angana Senpan, Xinmai Yang, Kwang H. Song, Mike J. Scott, Huiying Zhang, Patrick J. Gaffney, Samuel A. Wickline, Lihong V. Wang, and Gregory M. Lanza, Molecular Photoacoustic Tomography with Colloidal Nanobeacons, Angew. Chem. Int. Ed. 2009, 48, 4170-4173.
9. Jesse V. Jokerst, Mridhula Thangaraj, Paul J. Kempen, Robert Sinclair, and Sanjiv S. Gambhir, Photoacoustic Imaging of Mesenchymal Stem Cells in Living Mice via Silica-Coated Gold Nanorods, ACS Nano, 2012, 6, 5920-5930.
10. Jin-Woo Kim, Ekaterina I. Galanzha, Evgeny V. Shashkov, Hyung-Mo Moon and Vladimir P. Zharov, Golden carbon nanotubes as multimodal photoacoustic and photothermal high-contrast molecular agents, Nature Nanotechnology, 2009, 4, 688-694.
11. Kimberly A. Homan, Michael Souza, Ryan Truby, Geoffrey P. Luke, Christopher Green, Erika Vreeland, and Stanislav Emelianov, Silver Nanoplate Contrast Agents for in Vivo Molecular Photoacoustic Imaging, ACS Nano, 2012, 6, 641-650.
12. Geng Ku, Min Zhou, Shaoli Song, Qian Huang, John Hazle, and Chun Li, Copper Sulfide Nanoparticles As a New Class of Photoacoustic Contrast Agent for Deep Tissue Imaging at 1064 nm, ACS Nano, 2012, 6, 7489-7496.
13. Dipanjan Pan, Xin Cai, Ceren Yalaz, Angana Senpan, Karthik Omanakuttan, Samuel A. Wickline, Lihong V. Wang, and Gregory M. Lanza, hotoacoustic Sentinel Lymph Node Imaging with Self-Assembled Copper Neodecanoate Nanoparticles, ACS Nano, 2012, 6, 1260-1267
14. Adam De La Zerda, Cristina Zavaleta, Shay Keren, Srikant Vaithilingam, Sunil Bodapati, Zhuang Liu, Jelena Levi, Bryan R. Smith, Te-Jen Ma, Omer Oralkan, Zhen Cheng, Xiaoyuan Chen, Hongjie Dai, Butrus T. Khuri-Yakub And Sanjiv S. Gambhir, Carbon Nanotubes As Photoacoustic Molecular Imaging Agents In Living Mice, Nature nanotechnology, 2008, 3, 557-562.
15. Magdalena Swierczewska, Ki Young Choi, Edward L. Mertz, Xinglu Huang, Fan Zhang, Lei Zhu, Hong Yeol Yoon, Jae Hyung Park, Ashwinkumar Bhirde, Seulki Lee, and Xiaoyuan Chen, A Facile, One-Step Nanocarbon Functionalization for Biomedical Applications, Nano Lett., 2012, 12, 3613-3620
16. Chao Wang, Xinxing Ma, Shuoqi Ye, Liang Cheng, Kai Yang, Liang Guo, Changhui Li, Yonggang Li, and Zhuang Liu, Protamine Functionalized Single-Walled Carbon Nanotubes for Stem Cell Labeling and In Vivo Raman/Magnetic Resonance/Photoacoustic Triple-Modal Imaging, Adv. Funct. Mater., 2012, 22, 2363-2375
17. Chulhong Kim, Christopher Favazza, and Lihong V. Wang*, In Vivo Photoacoustic Tomography of Chemicals: High-Resolution Functional and Molecular Optical Imaging at New Depths, Chem. Rev. 2010, 110, 2756-2782
18. Jonathan F. Lovell, Cheng S. Jin, Elizabeth Huynh, Honglin Jin, Chulhong Kim, John L. Rubinstein, Warren C. W. Chan, Weiguo Cao, Lihong V. Wang and Gang Zheng Porphysome nanovesicles generated by porphyrin bilayers for use as multimodal biophotonic contrast agents, Nature Materials, 2011, 10, 324-332.
19. Elizabeth Huynh, Jonathan F. Lovell, Brandon L. Helfield, Mansik Jeon, Chulhong Kim, David E. Goertz, Brian C. Wilson, and Gang Zheng, Porphyrin Shell Microbubbles with Intrinsic Ultrasound and Photoacoustic Properties, J. Am. Chem. Soc. 2012, 134, 16464-16467.
20. John D. Simon,* Lian Hong, and Dana N. Peles, Insights into Melanosomes and Melanin from Some Interesting Spatial and Temporal Properties, J. Phys. Chem. B 2008, 112, 13201-13217
21. Hill, H. Z. BioEssays 1992, 14, 49-56.
22. Simon, J. D. Acc. Chem. Res. 2000, 33, 307-313.
23. Meredith, P.; Sarna, T. Pigm. Cell Res. 2006, 19, 572-594.
24. R. M. J. INGS, The Melanin Binding of Drugs and Its Implications, Drug Metabolism Reviews 1984, 15, 1183-1212
25. Boulton, M., Rozanowska, M. & Rozanowski, B. (2001) J. Photochem. Photobiol. B 64, 144-161.
26. Sarna, T. (1992) J. Photochem. Photobiol. B 12, 215-258.
27. Cheng Z, Mahmood A, Li H, Davison A, Jones AG. [99 mTcOAADT]-(CH2)2-NEt2: a potential small-molecule single-photon emission computed tomography probe for imaging metastatic melanoma. Cancer Res. 2005; 65:4979-4986.
28. Cheng Z, Zhang L, Graves E, et al. Small-animal PET of melanocortin 1 receptor expression using a 18F-labeled alpha-melanocyte-stimulating hormone analog. J Nucl Med. 2007, 48, 987-994.
29. Gang Ren, Zheng Miao, Hongguang Liu, Lei Jiang, Naengnoi Limpa-Amara, Ashfaq Mahmood, Sanjiv S. Gambhir, and Zhen Cheng, Melanin-Targeted Preclinical PET Imaging of Melanoma Metastasis, THE JOURNAL OF NUCLEAR MEDICINE, 2009, 50, 1692-1699.
30. Susan E. Forest and John D. Simon, Wavelength-dependent Photoacoustic calorimetry Study of Melanin, Photochemistry and Photobiology, 1998, 68, 296-298.
31. Chunxia Qin, Kai Cheng, Kai Chen, Xiang Hu, Yang Liu, Xiaoli Lan, Yongxue Zhang, Hongguang Liu, Yingding Xu, Lihong Bu, Xinhui Su, Xiaohua Zhu, Shuxian Meng & Zhen Cheng, Tyrosinase as a multifunctional reporter gene for Photoacoustic/MRI/PET triple modality molecular imaging, SCIENTIFIC REPORTS, 2013, 3, DOI: 10.1038/srep01490
32. Arie Krumholza, Sarah Chavezb, Junjie Yaoa, Timothy Flemingb, William E. Gillandersb, Lihong V. Wang, Tyrosinase-catalyzed melanin as a contrast agent for photoacoustic tomography, Proc. of SPIE, 2011, 7899, 78991G-1-6.
33. Kuk-Youn Ju, Yuwon Lee, Sanghee Lee, Seung Bum Park, and Jin-Kyu Lee, Bioinspired Polymerization of Dopamine to Generate Melanin-Like Nanoparticles Having an Excellent Free-Radical-Scavenging Property, Biomacromolecules 2011, 12, 625-632.
34. Brandon-Luke L. Seagle, Kourous A. Rezai, Yasuhiro Kobori, Elzbieta M. Gasyna, Kasra A. Rezaei, and James R. Norris, Jr., Melanin photoprotection in the human retinal pigment epithelium and its correlation with light-induced cell apoptosis, PNAS, 2005, 102, 8978-8983.
35. Haeshin Lee et al., Mussel-Inspired Surface Chemistry for Multifunctional Coatings, Science, 2007, 318, 426-430
36. Yun-Sheng Chen, Wolfgang Frey, Salavat Aglyamov, and Stanislav Emelianov, Environment-Dependent Generation of Photoacoustic Waves from Plasmonic Nanoparticles, Small 2012, 8, 47-52

### Example 2

### Brief Introduction

Developing multifunctional, biocompatible and easily prepared nanoplatforms with integrated different modalities is highly challenging for molecular imaging. Here, we report the successful transferring an important imaging biomarker, melanin, into a novel multimodality imaging nanoplatform. The multifunctional biopolymer nanoplatform based on water-soluble melanin nanoparticle (MNP) was developed and showed unique photoacoustic property and natural binding ability with metal ions (for example, ⁶⁴Cu²⁺, Fe³⁺). Therefore MNP not only can serve as a photoacoustic contrast agent, but also be used as a nanoplatform for positron emission tomography and magnetic resonance imaging. Traditional passive nanoplatforms require complicated and time-consuming processing for pre-building reporting moieties or require chemical modifications using active groups to integrate different contrast properties into one entity. In comparison, utilizing functional biomarker melanin can greatly simplify the building process. The multimodal properties of MNPs demonstrate the high potential of endogenous materials with multifunctions as nanoplatforms for clinical translation.

### Introduction:

Naturally produced biopolymers in living organisms play crucial roles in materials discovery and development. They have inspired scientists to synthesize novel biomaterials through mimicking Mother Nature, or they can further serve as templates and building blocks to prepare new generations of biocompatible, bioregenerative, or biodegradable materials for biomedical applications. For instance, DNA has been used to rationally design plasmonic nanostructures¹, to build nanoscaffolds for incorporating multiple-affinity ligands², and to self-assemble into numerous prescribed 3D shapes'. Cellular membranes have also been widely imitated by phospholipids and polysaccharides to form liposome or micelles for drug and imaging agent delivery^{4, 5}. Leukocytes membranes have also been used to coat silicon nanoparticles (NPs) to yield hybrid NPs which achieve cell-like functions including avoiding clearance by the immune system⁶. All these studies highlight the power of biomimicry for development of novel biomaterials.

Multimodal imaging combines different modalities together to provide complementary information and achieve synergistic advantages over any single modality alone. It has emerged as a very promising strategy for pre-clinical research and clinical applications⁷. One major challenge of multimodal imaging is to develop an efficient platform to load various components with individual contrast properties together whilst maintaining compact size, good biocompatibility and targeting capability. A variety of nanomaterials have been explored for multimodal imaging. In particular, exogenous inorganic NPs-based reporters have attracted considerable interests⁸⁻¹¹, such as iron oxide NPs for magnetic resonance imaging (MRI) and quantum dots for fluorescence imaging. Compared with inorganic NPs, organic NPs generally exhibit good biocompatibilities, biodistribution and clearance although most of them only appear to possess optical imaging properties¹². Some biomolecules based NPs such as liposomes have been widely used for loading contrast agents and drugs. But they themselves lack intrinsic contrast properties and only function as carriers. Therefore such biomolecules need complicated and time-consuming processes to pre-build various contrast properties or require chemical modifications to integrate different reporting moieties into one entity, which we term as a passive platform. For example, organic ligands are generally incorporated into a nanoplatform before chelating to radioactive or magnetic metal ions for positron emission tomography (PET)¹³ and MRI¹⁴.

Melanin, an amorphous, irregular functional biopolymer and a ubiquitous natural pigment which presents in many organisms including human skin, is a typical biomarker for disease imaging including melanoma detection and Parkinson Diseases diagnosis¹⁵⁻¹⁷. In this study, we report the successful transferring of this biomarker into an imaging nanoplatform. By mimicking natural melanin, water-soluble melanin nanoparticle (MNP) has been synthesized and used as the active platform for multimodal imaging of tumors. We demonstrate that MNP can not only offer its native optical properties for photoacoustic imaging (PAI), but also actively chelate to metal ions (⁶⁴Cu²⁺, Fe³⁺) for PET/MRI with a high loading capacity and stability utilizing its intrinsic chelating function. Furthermore, ultrasmall size MNP (˜4.5 nm) can be easily prepared and showed favorable in vivo pharmacokinetics and tumor targeting ability. Overall, these unique properties significantly simplify the process of preparation of multimodal imaging probes and make MNP a highly promising nanomaterial for biomedical applications.

### Results

### Synthesis and Characterization of MNPs

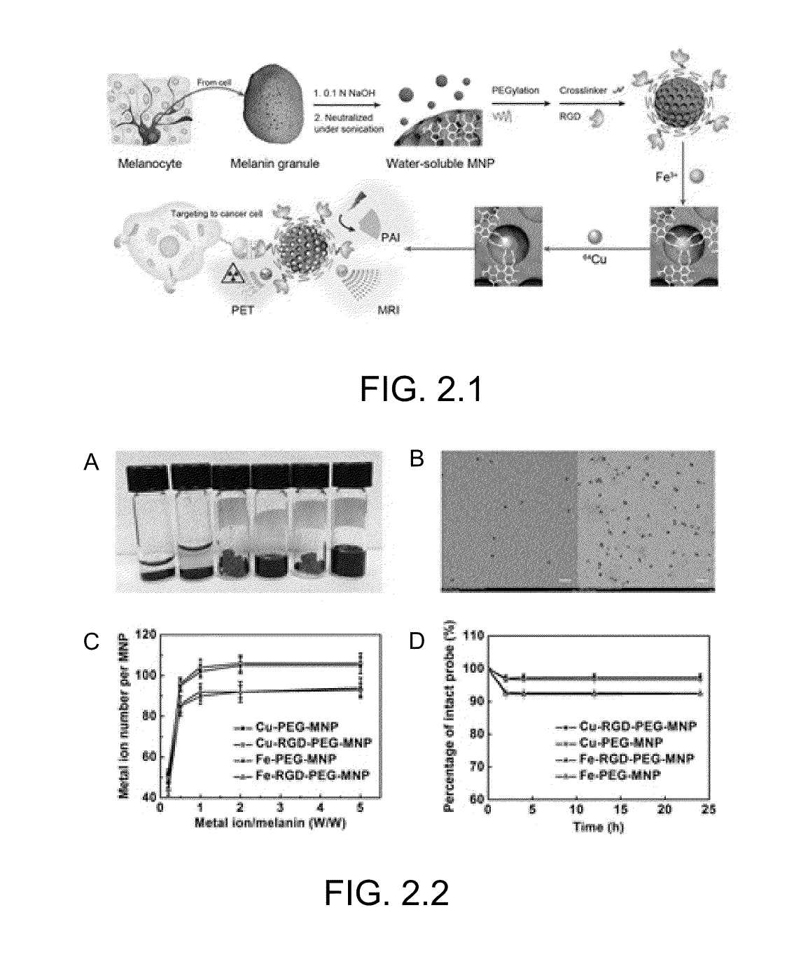

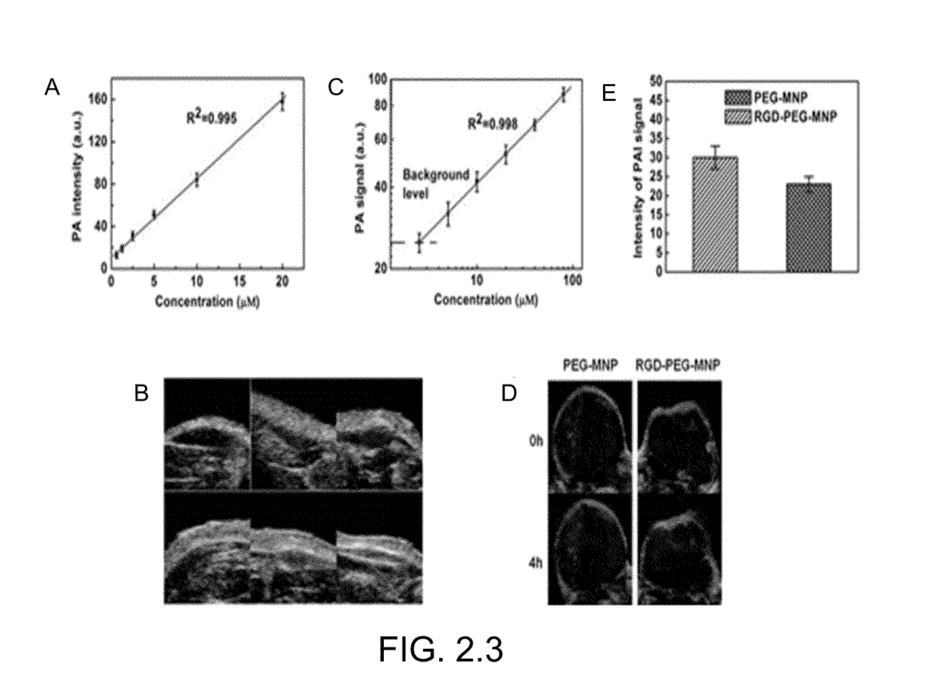

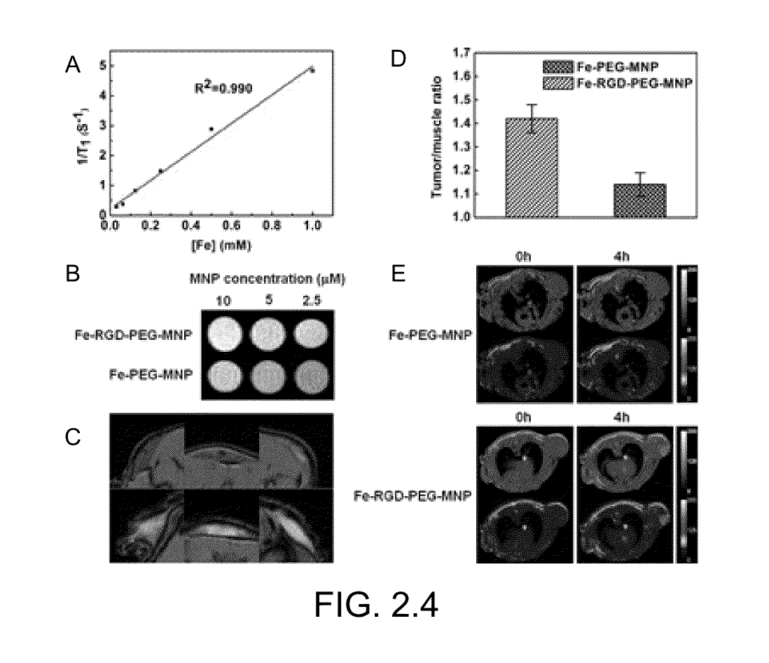

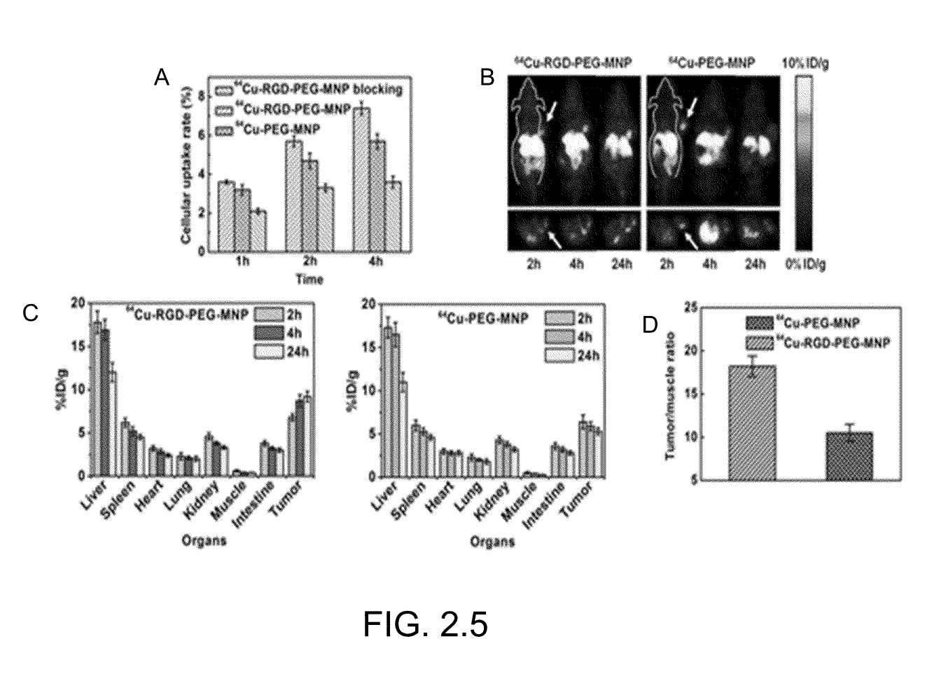

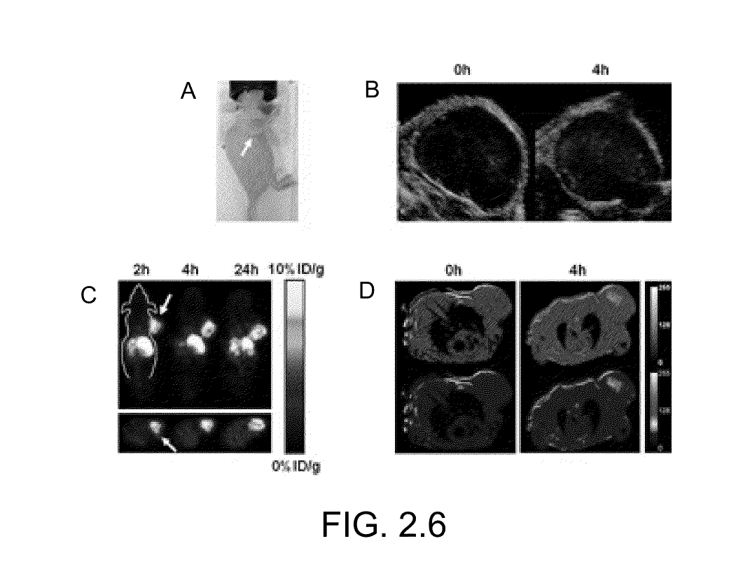

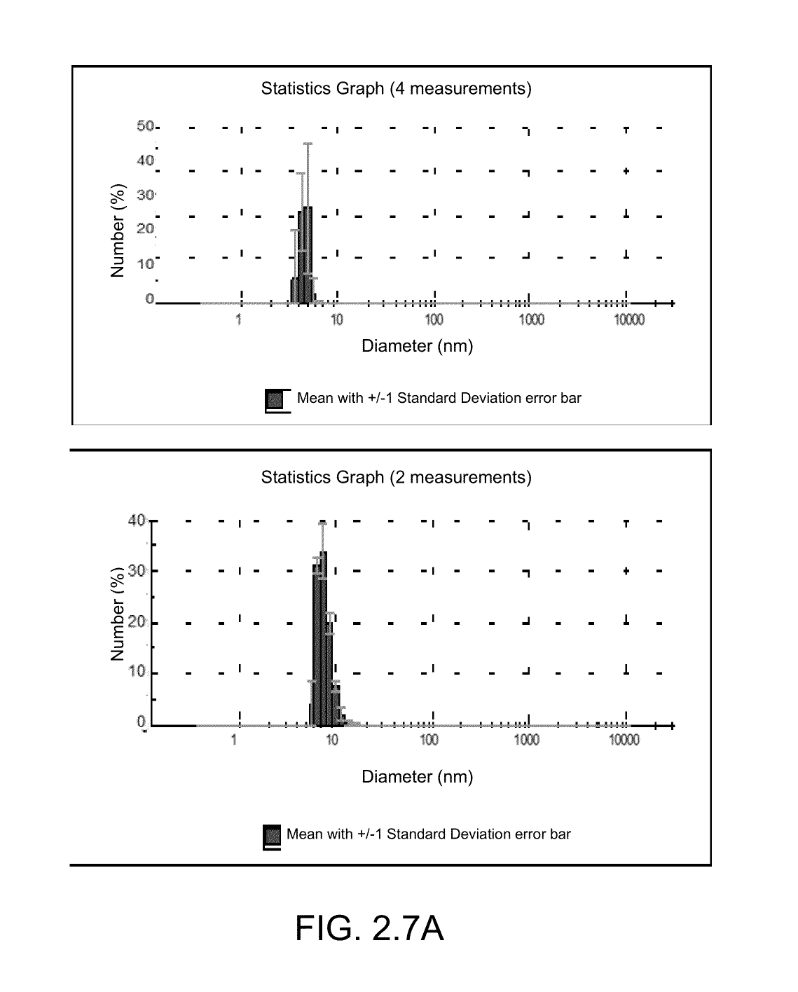

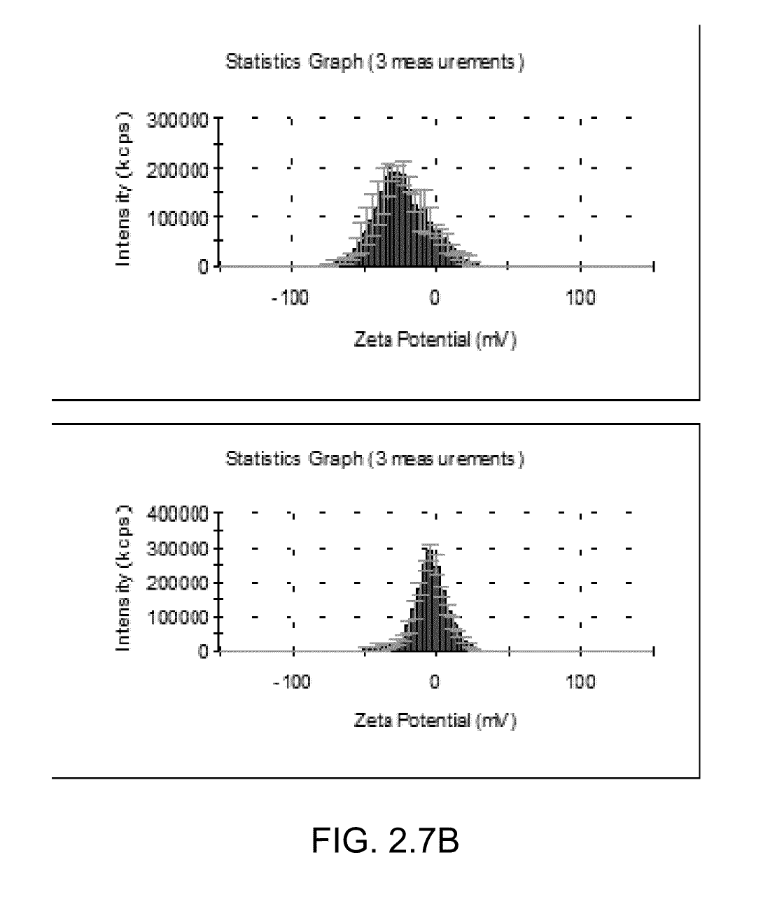

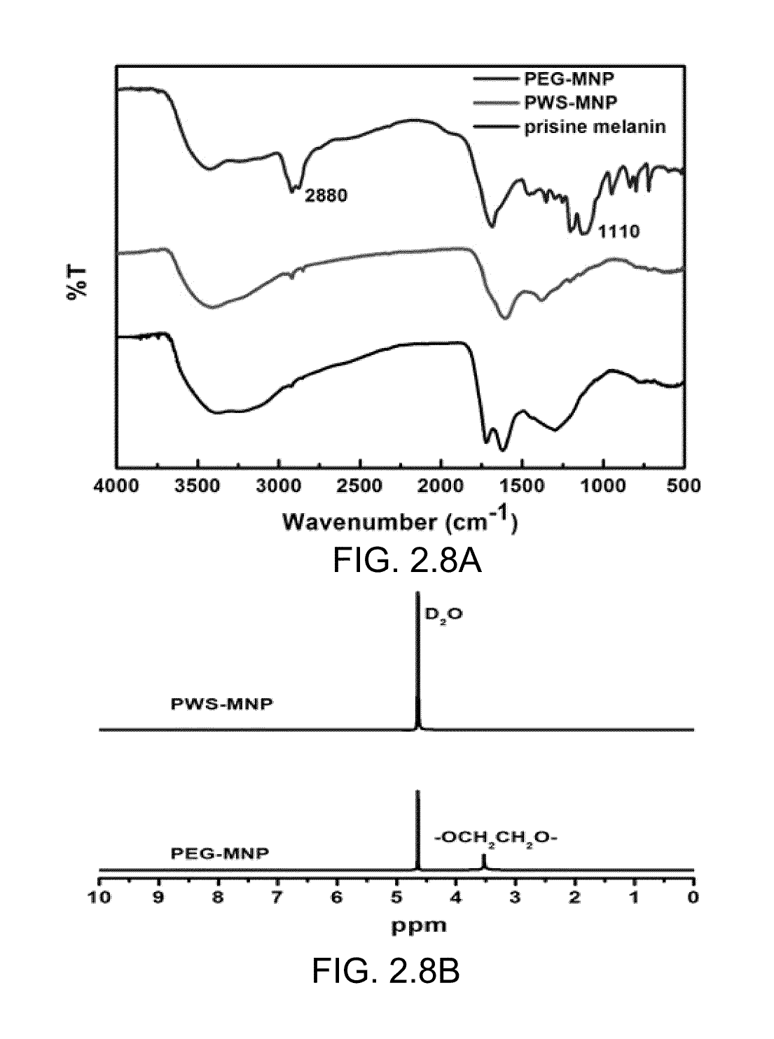

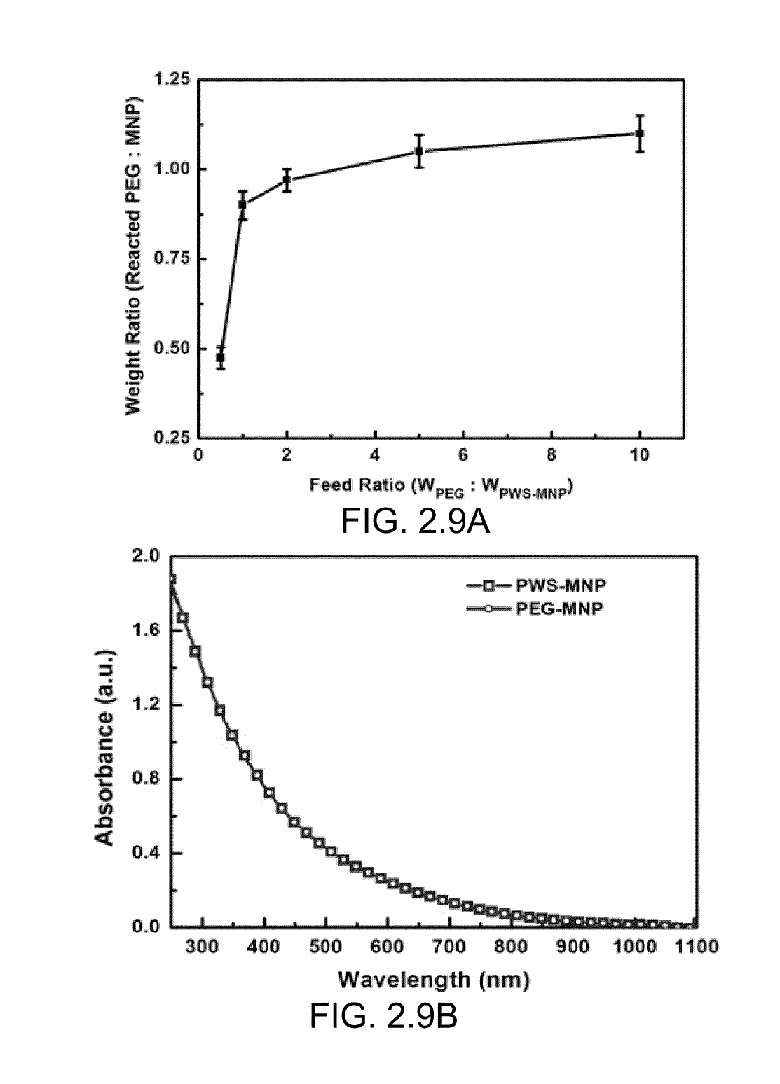

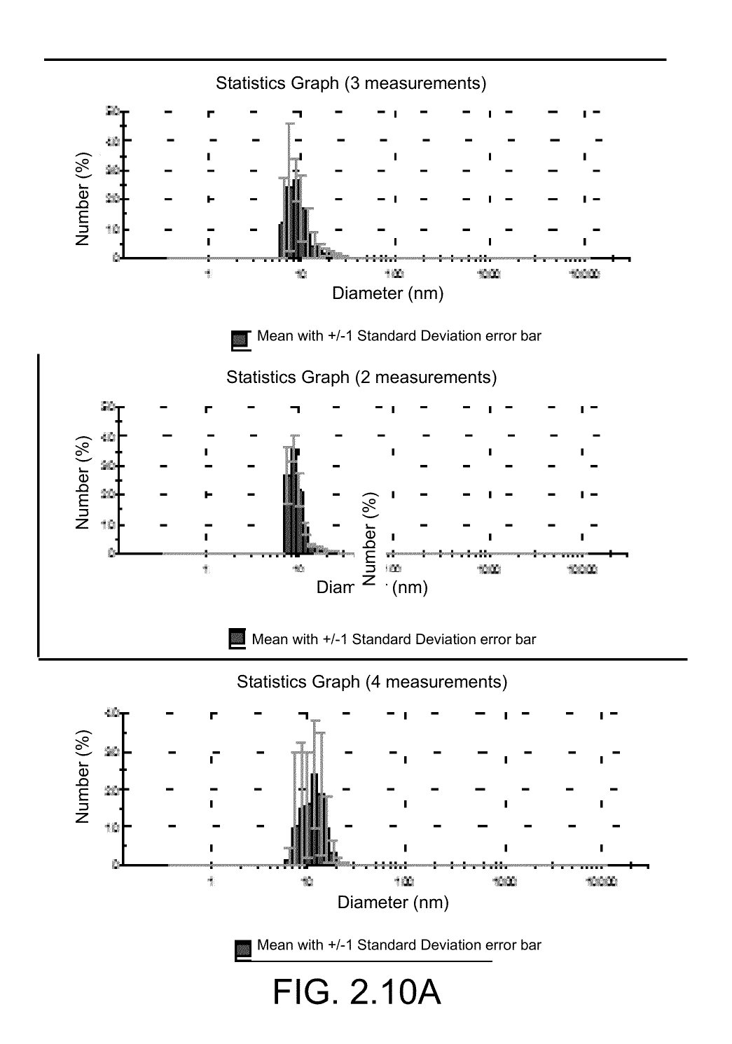

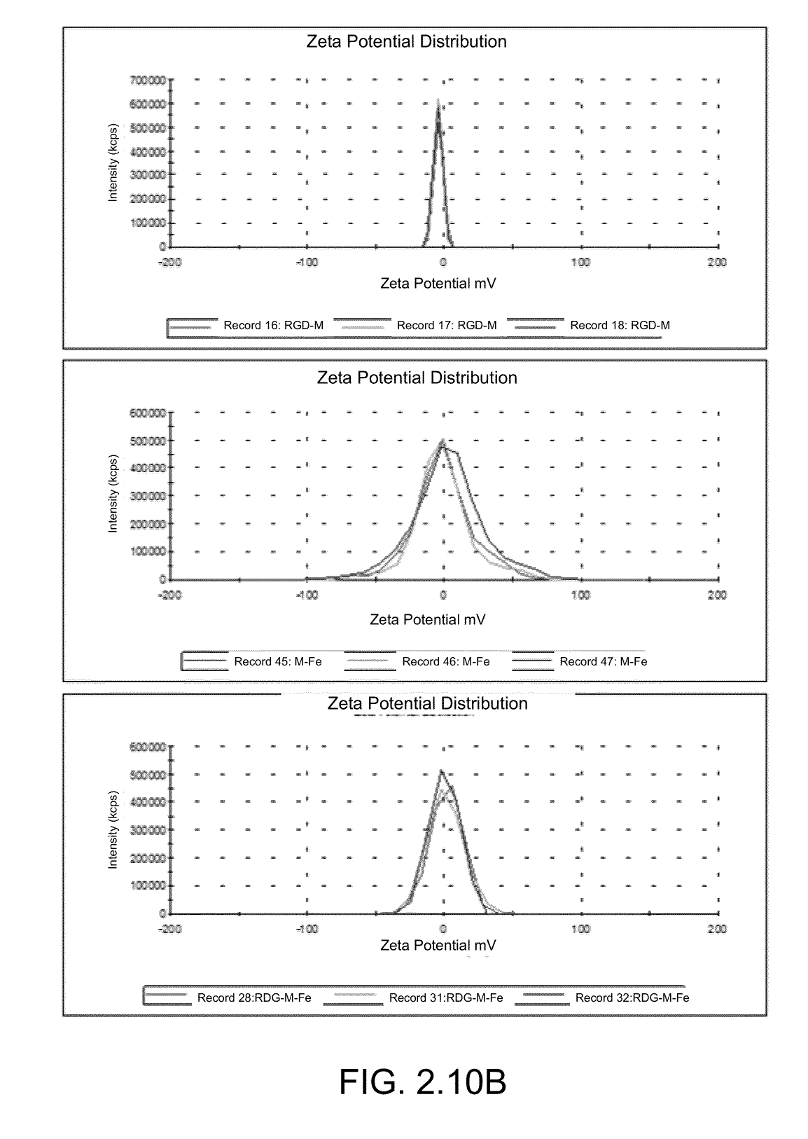

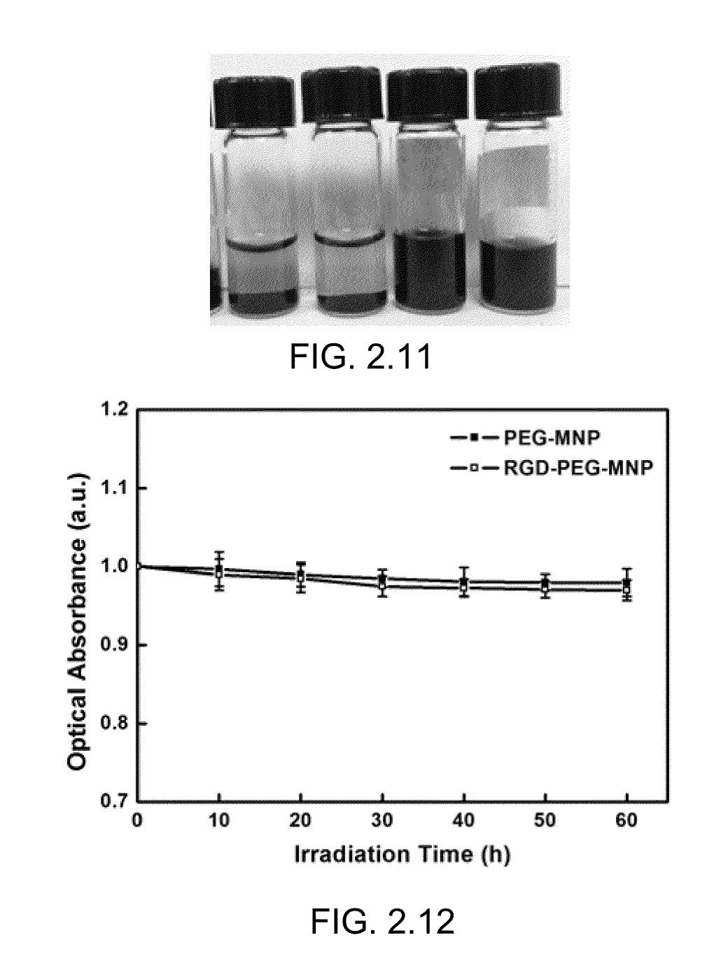

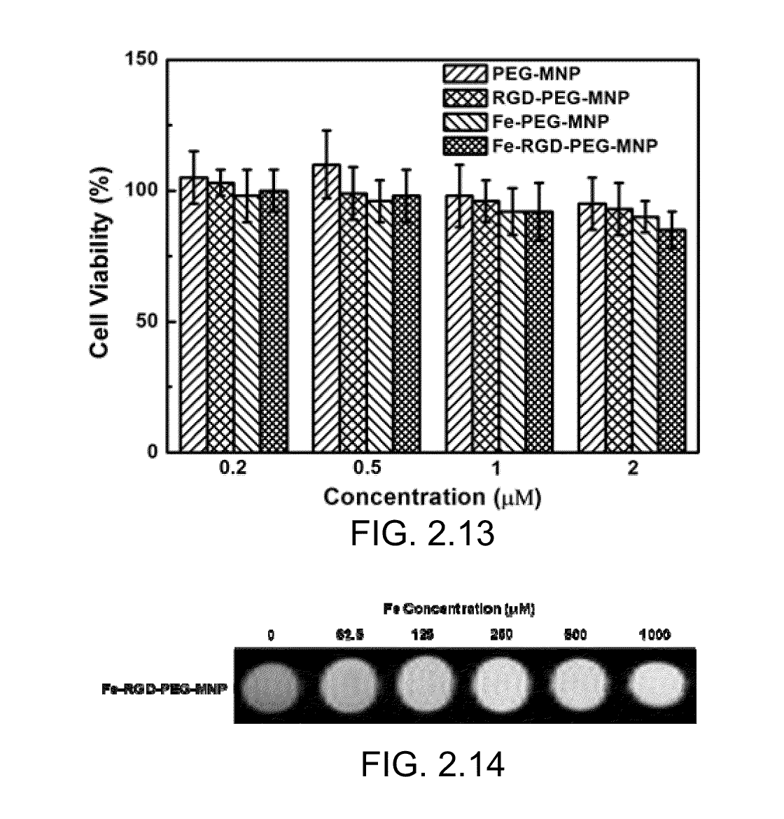

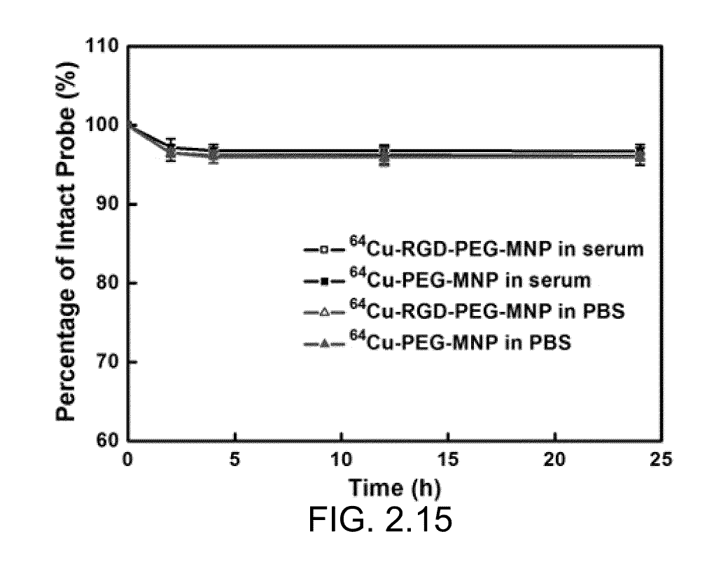

FIG. 2.1 schematically illustrates the procedure to prepare ultrasmall water-soluble MNP with multimodal imaging properties. To change the intrinsic poor water-solubility of melanin, pristine melanin granule was firstly dissolved in a 0.1 N NaOH¹⁸ and then neutralized under the assistance of sonication to decrease interchain aggregation. Ultrasmall MNP in high water monodispersity and homogeneity with a size of 4.5±0.3 nm, which was termed as plain water-soluble MNP (PWS-MNP), were successfully obtained (FIG. 2.2a, 2.2b and FIG. 2.7A). PWS-MNP exhibited excellent water-solubility of 40 mg/mL and stability which can be attributed to the highly negative potential of approximately −22.5 mV on the NP surface that efficiently blocks the NP aggregation through electrostatic repulsion (FIG. 2.7B). Furthermore, PWS-MNP can be stored as lyophilized powder for over six months and effectively re-dissolved in water allowing long-term usage (FIG. 2.2a). The FT-IR spectra of pristine melanin granule and PWS-MNP were similar to each other, indicating no significant change of molecular structure (FIG. 2.8A). The ¹H NMR spectrum of PWS-MNP in D₂O showed no obvious signal belonging to the hydrogen atom on the arylene groups, suggesting most of the conjugated backbones were buried in the NP¹⁹ (FIG. 2.8B). The molecular weight of a PWS-MNP was calculated from the nanoparticle size and its density (1.3 g/cm³) which is about 40 kDa.

To further enhance the biocompatibility of PWS-MNPs, polyethyleneglycol (PEG) chains²⁰ were introduced to the MNP. NH₂—PEG₅₀₀₀-NH₂ was used because the amine groups can react with dihydroxyindole/indolequinone groups in melanin²¹. The number of PEG chains per MNP was determined to be about 10 (FIG. 2.9A). The diameter of the PEG-functionalized MNP (PEG-MNP) became large and reached 7.0 nm (FIG. 2.2b and FIG. 2.7A). Moreover, the surface potential of PEG-MNP decreased to −4.5 mV (FIG. 2.7B) because of introduction of PEG and positive NH₂ groups on the MNP surface. The similar absorption spectrum of PEG-MNP to PWS-MNP demonstrated that the PEG-modification did not influence the absorption properties of melanin (FIG. 2.9B). Lastly, for demonstrating that MNP can be used as a platform for tumor targeting, PEG-MNP was further modified with biomolecules such as cyclic Arg-Gly-Asp-d-phe-Cys [c(RGDfC)] peptide (abbreviated as RGD) which can target tumor αᵥβ₃ integrin²². The number of RGD attached to the MNP was calculated to be about 8 per MNP and the size of RGD-functionalized PEG-MNP (RGD-PEG-MNP) increased a little to ˜8.7 nm (FIG. 2.10).

### Chelating to Cu²⁺ and Fe³⁺

To investigate the possibility of MNP as a platform for PET and MRI, its chelating properties to Cu²⁺ (⁶⁴Cu²⁺ for PET) and Fe³⁺ (for MRI) were studied. After adding metal ions (0.2 mL of 10 mM FeCl₃ or CuCl₂) into MNP aqueous solutions (1 mL of 20 μM for PWS-MNP and PEG-MNP), the precipitation of PWS-MNP quickly appeared while PEG-MNP maintained good water-solubility (FIG. 2.11). The Fe³⁺ or Cu²⁺-chelated MNP (Fe-PEG-MNP, Fe-RGD-PEG-MNP, Cu-PEG-MNP and Cu-RGD-PEG-MNP) exhibited high loading capacities. The maximum quantities of one MNP to chelate to Cu²⁺ and Fe³⁺ are about 100 and 90 ions respectively, no matter whether RGD is attached to the MNP or not (FIG. 2.2c, considering the requirement of remaining chelating positions on the Fe³⁺-chelated MNP for further chelating to ⁶⁴Cu²⁺, Fe-PEG-MNP and Fe-RGD-PEG-MNP with ˜50 Fe ions were used for all following studies). After Fe³⁺-chelating, the MNP sizes increased to ˜9.0 nm and ˜11.2 nm for Fe-PEG-MNP and Fe-RGD-PEG-MNP respectively and their zeta-potential remained in the neutral region (FIG. 2.10 and Table 1).

### Stability and Biocompatibility of MNPs

The optical stabilities of PEG-MNP and RGD-PEG-MNP under increasing durations of light exposure were further tested. Compared with those reported dyes for PAI which exhibit significant reduced absorption (>30%) under light exposure²³, PEG-MNP and RGD-PEG-MNP showed intriguing photo-stability (only 3% reduced absorption) (FIG. 2.12), indicating their high capability for PAI. Further stability assay of Fe³⁺ or Cu²⁺-chelated RGD-PEG-MNP and PEG-MNP in mouse serum showed that only 3% Cu²⁺ and 7% Fe³⁺ were released from those MNPs at the first 2 h, and there was no further release at longer incubation time points, indicating the high stability of the chelating platform (FIG. 2.2d). The first 2 h released metal ions may derive from those which were absorbed on the MNPs through weak electrostatic interaction. Furthermore, the high viability of NIH3T3 cells (85-105% as compared to the nontoxic control) after 24 h of incubation with PEG-functionalized MNPs was found, indicating high biocompatibility and low cytotoxic effect of PEG-functionalized MNPs (FIG. 2.13).

### PAI of MNPs

To investigate the possibility of MNPs to be used as a photoacoustic agent, we firstly studied the detection sensitivity of PEG-MNP in aqueous solution at increasing concentrations from 0.625 to 20 μM. The PEG-MNP with 0.625 μM was detected, and the photoacoustic signals increased linearly with the increase of PEG-MNP concentrations (R²=0.995) (FIG. 2.3a).

The detection sensitivity of MNP in living body was further tested by subcutaneous injection of PEG-MNP on the lower back of mice (n=3) at increasing concentrations of 5 to 80 μM (FIG. 2.3b). A linear correlation (R²=0.998) between the MNP concentration and the corresponding photoacoustic signal was observed in FIG. 2.3c. The background signal from tissue was quantified using the signals from the areas without injection any contrast agent. 2.5 μM of PEG-MNP was found to give the equivalent photoacuoustic signal strength as the tissue background.

To further investigate their in vivo PAI properties, two groups of U87MG tumor mice were tail-vein injected with 250 μL of either PEG-MNP or RGD-PEG-MNP at a concentration of 200 μM. Mice showed obvious increase of photoacoustic signal in tumors after injection with RGD-PEG-MNP than that of PEG-MNP at 4 h (FIG. 2.3d). The increased photoacoustic signal of RGD-PEG-MNP was much higher than PEG-MNP (e.g., 31.5±2.8 vs. 23.0±1.9) in FIG. 2.3e, because of the tumor targeting ability of RGD-PEG-MNP to αᵥβ₃ integrin.

### MRI of MNPs

To study whether Fe³⁺ (T₁ contrast agent) retains MR signal-enhancing property after loading into MNPs, T₁-weighted MRI images of various concentrations of Fe-RGD-PEG-MNP in agarose gel was investigated (FIG. 2.14). With the increase of NP concentration, MR signal was significantly enhanced, suggesting Fe-RGD-PEG-MNP generate a high magnetic field gradient on their surface. R₁ value of Fe-RGD-PEG-MNP (the slope of the fitted curve in FIG. 2.4a) was calculated to be 4.8 mM⁻¹s⁻¹.

To investigate the MRI ability and sensitivity of Fe-RGD-PEG-MNP and Fe-PEG-MNP for cells, three different concentrations of the MNPs were used to incubate with U87MG cells overexpressing integrin αᵥβ₃. It was found that U87MG cells cultured with Fe-RGD-PEG-MNP displayed higher signals than that of Fe-PEG-MNP, indicating the RGD-moiety contributes to the MNP uptake by U87MG cells (FIG. 2.4b). The MR signal of U87MG cells also increased slightly along with the increased concentration of Fe-RGD-PEG-MNP.

The magnetic sensitivity in living mice was firstly tested by subcutaneous injection of Fe-RGD-PEG-MNP on the lower back of mice (n=3) at increasing concentrations of 1.25 to 20 μM. It was extrapolated that 1.25 μM of Fe-RGD-PEG-MNP produced the equivalent MRI signal intensity as the tissue background (FIG. 2.4c).

To demonstrate the use of MNP as the platform for MRI of tumors, T1-weighted images were obtained from mice bearing U87MG tumors (n=4 per group). U87MG tumors injected with Fe-RGD-PEG-MNP displayed higher signals compared with Fe-PEG-MNP at 4 h (FIG. 2.4e). The tumor to muscle ratio of MR signal intensity was 1.42±0.06 for Fe-RGD-PEG-MNP, which was significantly higher than 1.14±0.05 for Fe-PEG-MNP (P<0.05), demonstrating that MNP can be used as a platform for MRI (FIG. 2.4d).

### PET of MNPs

To investigate the PET imaging properties of MNP, ⁶⁴Cu was selected as a PET radiolabel for MNP because it can be readily chelated by melanin and the intermediate half-life of ⁶⁴Cu (12.7 hour) makes it suitable for radiolabeling of biomolecules and imaging²⁴⁻²⁶. Simple mixing of RGD-PEG-MNP and PEG-MNP with ⁶⁴Cu allowed successfully labeling the NPs in the yield of 80%. The resulting MNPs, ⁶⁴Cu-RGD-PEG-MNP and ⁶⁴Cu-PEG-MNP, displayed excellent stability in mouse serum and PBS solution (FIG. 2.15). Similar to Cu²⁺-chelated MNPs, only ˜4% ⁶⁴Cu released from the MNPs after 24 h of incubation. Thus, ⁶⁴Cu-labelled MNPs were easily and reliably produced and demonstrated high stability in vitro.

Uptake of ⁶⁴Cu-PEG-MNP and ⁶⁴Cu-RGD-PEG-MNP by U87MG cells with or without blocking agent RGD at 1, 2 and 4 h are shown in FIG. 2.5a. ⁶⁴Cu-RGD-PEG-MNP exhibited higher uptakes than ⁶⁴Cu-PEG-MNP at all the incubation time, with a value of 3.6%, 5.7% and 7.4% for ⁶⁴Cu-RGD-PEG-MNP and 3.2%, 4.7% and 5.7% for ⁶⁴Cu-PEG-MNP at 1, 2 and 4 h, respectively. In comparison, for ⁶⁴Cu-RGD-PEG-MNP blocking group, much lower uptake of ⁶⁴Cu-RGD-PEG-MNP was observed with a value of 2.1%, 3.3% and 3.6% at 1, 2 and 4 h, respectively, indicating the specific targeting ability of RGD contributes to the uptake of ⁶⁴Cu-RGD-PEG-MNP by U87MG cells.

The in vivo PET of MNPs was performed in U87MG-tumor-bearing mice. Both ⁶⁴Cu-RGD-PEG-MNP and ⁶⁴Cu-PEG-MNP showed tumor accumulation and clear tumor contrast after 2 h post-injection (FIG. 2.5b). Quantification analysis revealed that the tumor uptake values of ⁶⁴Cu-RGD-PEG-MNP increased with time to 24 h, and they were 6.8, 8.7, and 9.2% ID/g at 2, 4, and 24 h, respectively (FIG. 2.5c). As a comparison, the uptake of ⁶⁴Cu-PEG-MNP was the highest at 2 h and decreased with time and significantly much lower than ⁶⁴Cu-RGD-PEG-MNP (for example, 5.3% vs. 8.7%, at 4 h). After 4 h injection with ⁶⁴Cu-RGD-PEG-MNP, the tumor-to-muscle (T/M) ratio was about 18, which was also significantly higher than T/M ratio of 10 of ⁶⁴Cu-PEG-MNP (FIG. 2.5d). In addition to the tumor, moderate activity accumulation was observed in the liver (e.g., 11.0-12.0% ID/g at 24 h for all MNPs), and relative lower activity accumulation was also found in the kidneys (e.g., 3.2-3.3% ID/g at 24 h for all MNPs). These data indicated the MNP was cleared through both hepatobiliary and renal system.

### PAI/MRI/PET of ⁶⁴Cu—Fe-RGD-PEG-MNP

To investigate the possibility of using MNP platform for multimodality imaging, MNP were mixed with Fe³⁺ and ⁶⁴Cu in sequence to form the multifunctional probe ⁶⁴Cu—Fe-RGD-PEG-MNP for PAI/PET/MRI. PET, T1-weighted MRI and PAI of mice bearing U87MG tumors were then obtained sequentially. In FIG. 2.6, ⁶⁴Cu—Fe-RGD-PEG-MNP showed very similar PET and MRI properties on U87MG tumor, compared with the corresponding ⁶⁴Cu-RGD-PEG-MNP and Fe-RGD-PEG-MNP, respectively. These results showed that using MNP as the active platform to load ⁶⁴Cu²⁺ and Fe³⁺ together can efficiently combine its native photoacoustic properties with radioactive and magnetic properties together for multimodality imaging.

### Discussion

To change the lack of contrast properties of biomolecule-based nanoplatform for multimodality imaging, recently porphyrin were successfully introduced into phospholipid to provide the platform with desirable optical properties²⁷⁻²⁹ while it still requires complicated and time-consuming chemical modifications and other reporting molecules to achieve multimodality imaging ability. We herein develop the functional biomarker, melanin, as a novel nanoplatform with its native optical property and multifunctions which can simply and actively collecting optical, magnetic and radioactive properties together for multimodality imaging. Melanin, the oxidation products of tyrosine, plays an important role in living organism³⁰. Accompanied with the development of molecular imaging probes in the past decade, melanin has been used as an effective molecular target³¹⁻³³ as well as endogenous contrast agent for PAI because of its strong light absorption properties³⁴′³⁵. Besides, melanin has intrinsic strong chelating properties to many metal ions including Cu²⁺ and Fe^{3+ 36-38}, which can be used to nuclear imaging and MRI. Consequently melanotic melanomas shows hyperintensity on T1-weighted MRI images^{39, 40}.

Considering the attractive properties of melanin, we and others have engineered cancer cells to biologically produce melanin for multimodality imaging (PAI/MRI/PET) of cancer⁴¹⁻⁴³. However, this method requires genetic modification of cells, which is time-consuming and may have limited clinical value. Thus, water-soluble MNPs are more appropriate to behave as a natural “active platform” to simplify the preparation procedure for multimodal applications. Considering only trace amount of ⁶⁴Cu²⁺ ions utilized for PET and its final decay to Zn²⁺ ions which is necessary for life process, and the abundant amount of Fe³⁺ ions in living body, ⁶⁴Cu²⁺ and Fe³⁺ ions used in our system are expected to be metabolized in living subjects. Therefore, the ultrasmall MNP prepared has inherited high biocompatibility and biodegradability. More interestingly, this new NP can serve as an active nanoplatform and easily bind with metal ions without the needs of surface modification and introducing chelating groups, which significantly simplifies the preparation process and reduces the heterogeneity of the resulting multimodal NPs. Furthermore, the MNPs is an organic NPs with ultrasmall size, it can be cleared through both hepatobiliary and renal system and showed excellent tumor imaging properties (high tumor uptakes and high tumor to normal organ contrasts). All of these properties make MNPs are highly promising for clinical translation.

Despite its important functions, developing melanin for molecular imaging was highly subjected to its intrinsic poor water-solubility. Therefore preparing MNPs is desired for well-dispersing in water, especially for those with size around 10 nm that can provide appropriate blood circulation time. Although the formation mechanism of polymeric melanin is not clear, its molecular structure is generally considered to be composed of dihydroxyindole/indolequinone segments with hydrophobic conjugated main chain having strong π-π interaction and hydrophilic hydroxyl groups on the benzene rings⁴⁴. Therefore, to realize melanin water-soluble at neutral environments, decreasing the interchain π-π aggregation of conjugated main chain and lowering the formed melanin particle size to expose more hydrophilic hydroxy groups on the surface of melanin is a promising way. In our work, sonication was proved to be an efficient method to obtain ultrasmall MNP in water with high monodispersity and homogeneity. Another problem should be resolved is the metal ion-initiated crosslinking and the formation of precipitation. Recent reports showed that Fe³⁺ is a strong crosslinker for catechol groups⁴⁵. In our work, PEG encapsulation is found can not only enhance the biocompatibility and the water-solubility, but also efficiently prevent the formation of metal ion-initiated precipitation. Overall, a reliable method for preparation of water soluble MNP have been developed in our work, which lays down a foundation for its future biomedical applications. It can be easily envisioned that MNP can serve as a nanoplatform not only for molecular imaging but also for theranostics. Considering the abundant functionalities of melanin, such as binding drugs⁴⁶, MNP-based platform used for drug delivery and therapy are now being investigated.

### Conclusion

In conclusion, we report MNP as the first natural biomarker-transferred active platform for multimodality imaging. MNP is of particular interest because such an endogenous agent with native photoacoustic signals and strong chelating properties with metal ions can act as an active platform to simplify the assembling of different imaging moieties. MNP can be easily modified with biomolecules for targeted tumor multimodality imaging, and it showed excellent in vivo tumor imaging properties. We expect this work will stimulate further studies of multifunctional endogenous material as nanoplatforms for potential imaging and therapeutic applications.

### Methods

### Preparation of RGD-Conjugated MNPs.

Tyrosine-derived synthetic melanin was firstly dissolved in 0.1M KOH and then swiftly neutralized with 0.1 M HCl under sonication. A bright black PWS-MNP aqueous solution was obtained and purified with a centrifugal-filter (Amicon centrifugal filter device, MWCO=30 kDa). PWS-MNP was then functionalized with NH₂—PEG₅₀₀₀-NH₂ and the obtained PEG-MNP was conjugated with 4-(N-maleimidomethyl)cyclohexane-1-carboxylic acid 3-sulfo-N-hydroxysuccinimide ester sodium salt (sulfo-SMCC) and further cRGDfC to obtain RGD-PEG-MNP. ¹H-NMR spectra for MNPs were recorded on a 400 MHz NMR spectrometer (Bruker). FT-IR spectra were measured on a Bio-Rad FT-IR spectrophotometer (Model FTS135). The MNP sizes were determined by Transmission electron microscopy (TEM, JEOL 2010). The hydrodynamic sizes and Zeta potentials were measured using dynamic light scattering and zeta potential analyzer (Malvern, Zetasizer Nano ZS90).

### Preparation of Fe³⁺, Cu²⁺-Chelated and ⁶⁴Cu²⁺-Radiolabeled MNPs.

The MNPs were chelated to Fe³⁺ or Cu²⁺ by simply addition of FeCl₃ or CuCl₂ in buffer solution of pH=5.5 followed by a 1 h incubation at 40° C. The Fe³⁺ and Cu²⁺ concentrations of MNPs were measured by inductively coupled plasma-mass spectrometry (ICP-MS) analysis. MNPs with or without Fe³⁺ were further radiolabeled with ⁶⁴Cu²⁺ by addition of ⁶⁴CuCl₂ in 0.1 N NaOAc (pH 5.5) buffer followed by 1 h incubation at 40° C.

### Cell Viability and In Vitro Cell Uptake.

In vitro cytotoxicity of MNPs was determined in NIH-3T3 cells by the MTT assay. For PET analysis of cell uptake, U87MG cells were incubated with ⁶⁴Cu-labeled MNP in serum-free DMEM. The specific binding of the probes with U87MG cells was determined by co-incubation with RGD. After 1, 2, and 4 h, the cells were collected and their radioactivity was counted using a PerkinElmer 1470 automatic gamma-counter. For MRI analysis, U87MG cells were incubated with different concentration of Fe-chelated MNP in serum-free DMEM. After 4 h, the cells were collected and emerged in 1% agarose gel. T1 MRI was performed using a Siemens 1.0 T instrument. The imaging protocol consisted of localizer and axial T1-weighted fast spin echo (FSE) sequence with repetition time (TR): 700 ms and echo time (TE): 5.5 ms.

### PAI and MRI Analysis of Phantom.

For studying the PA properties of MNPs, different concentrations of MNP aqueous solutions were filled into polyethylene capillaries and then emerged in agarose gel. For the particles' sensitivity in living body, MNPs with different concentrations were mixed with matrigel and then subcutaneously injected on the lower back of mice. The PAIs of the mixtures were collected after they were solidified. The Vevo LAZR PAI System (VisualSonics Inc., Toronto, Canada) with a laser at excitation wavelength of 680 nm and a focal depth of 10 mm was used to acquire photoacoustic and ultrasound images. For studying the magnetic properties of MNPs, different concentrations of MNPs were filled into 1% agarose gel and placed into the MR scanner, and a number of MR sequences were run, spin-echo for R₁ determination (TR: 50-3000 ms; TE: 5.5 ms). For the magnetic sensitivity in living subject, Fe-chelated MNPs with different concentrations were also mixed with matrigel at 0° C. and then subcutaneous injected on the lower back of mice. The imaging protocol is the same as that for cell uptake investigation.

### PAI, MRI and PET of Tumor Bearing Mice.

All animal experiments were performed in compliance with the Guidelines for the Care and Use of Research Animals established by the Stanford University Animal Studies Committee. U87MG cells were inoculated subcutaneously in the shoulder of Female athymic nude mice in 4-6 weeks old. When the tumors reached 0.5-0.8 cm in diameter, the tumor bearing mice were subjected to in vivo multimodality imaging. Mice bearing U87-MG tumors were injected with Fe-labeled MNPs via the tail vein. After 4 h, MRI was performed using the same instrument, protocols and conditions as in the phantom MRI study. Imaging analysis was performed using the OsiriX software. T1 values of regions of interest (ROIs) drawn over the tumor and muscle were then measured, and the ratio of tumor/muscle was calculated. PAI was carried out using the same Vevo LAZR PAI System as the in vitro study. Similarly, image analysis was carried out using ImageJ, and quantification analysis was performed on the PAI images. Small animal PET imaging was performed on a Siemens Inveon microPET-CT. Mice bearing U87-MG tumors were tail-vein injected with ⁶⁴Cu-labeled MNPs. At different times after injection (2, 4 and 24 h), the mice were scanned by three-minute static scans. All PET images were reconstructed by two dimensional ordered-subsets expectation maximization (OSEM) algorithm. The radioactivity uptake in the tumor and normal tissues was calculated using ROIs drawn over the whole organ region and expressed as a percentage of the injected radioactive dose per gram of tissue (% ID/g).

### Detailed Methods

### Materials

The following reagents were acquired and used as received: melanin (Sigma Aldrich), sodium hydroxide (Sigma Aldrich), hydrochloric acid (37 wt %, Sigma Aldrich), NH₄OH solution (28 wt %, Sigma Aldrich), amine-PEG₅₀₀₀-amine (NH₂—PEG₅₀₀₀-NH₂, SkDa, Laysan Bio), dimethylthiazolyl-diphenyltetrazolium (MTT; Biotium), phosphate buffered saline (PBS, Gibco), and agarose (Invitrogen). Millipore water (at 18 MOhm) was used.

### Experiments

### Preparation of PWS-MNP.

Tyrosine-derived synthetic melanin (20 mg) was firstly dissolved in 10 mL 0.1N NaOH aqueous solution under vigorous stirring. After dissolving, HCl aqueous solution (0.1 N) was swiftly dropped into the obtained basic melanin solution to adjust the pH to 7 under sonication with output power=10 W for 1 min. A bright black melanin aqueous solution was obtained. The neutralized solution was further centrifuged with a centrifugal-filter (Amicon centrifugal filter device, MWCO=30 kDa) and washed with deionized water and repeated several times to remove the produced NaCl. The aqueous solvent was removed by freeze-drying to obtain 15 mg black solid of PWS-MNP.

Surface Modification of MNP with NH₂—PEG₅₀₀₀-NH₂ (PEG-MNP).

NH₄OH solution (28 wt %) was added to 5 mL of PWS-MNP aqueous solution (1 mg/mL of water) to adjust the pH of the solution to 9. This mixed solution was added dropwise into NH₂—PEG₅₀₀₀-NH₂ (5 mg, 10 mg, 25 mg, and 50 mg) aqueous solution with pH=9. After vigorous stirring for 12 h, PEG-modified MNP was retrieved by centrifugation with a centrifugal-filter (Amicon centrifugal filter device, MWCO=30 kDa) and washed with deionized water several times by redispersion/centrifugation processes to remove the unreacted NH₂—PEG₅₀₀₀-NH₂. The aqueous solvent was removed by freeze-drying and the PEG-MNP was weighed to calculate the quantity of the PEG attached on MNPs.

Conjugation of PEG-MNP with RGD (RGD-PEG-MNPs).

The crosslinker solution was prepared freshly. The 4-(N-maleimidomethyl)cyclohexane-1-carboxylic acid 3-sulfo-N-hydroxysuccinimide ester sodium salt (sulfo-SMCC) (1.2 mg) was firstly dissolved in 36 μL of dimethylsulfoxide (DMSO). The water-soluble PEG-MNP [1 mg in 1 mL PBS (pH=7.2)] was incubated with the above crosslinker solution for 2 h at room temperature. The resultant thiol-active MNP ran through a PD-10 column pre-washed with PBS (pH=7.2, 10 mM) to remove the excessive sulfo-SMCC and by-products. The purified MNP was concentrated to the final volume of 0.5 mL with a centrifugal-filter (MWCO=30 kDa). The cRGDfC stock solution (120 μL of 5 mM in the degassed water) was added to the above MNP solution with stirring. The conjugation reaction proceeded for 24 h at 4° C. The uncoupled RGD peptide was removed through a PD-10 column and collected to analyze its quantity through HPLC. The number of coupled RGD on one MNP was then calculated. The resultant product, RGD-PEG-MNP, were concentrated by a centrifugal-filter (MWCO=30 kDa) and stored at 4° C. for one month without losing targeting activity. The final RGD-PEG-MNP were reconstituted in PBS and filtered through a 0.22 μm filter for cell and animal experiments.

### Preparation of Fe³⁺ or Cu²⁺ Chelated RGD-PEG-MNPs and PEG-MNPs.

The MNP (1 mg in 1 mL H₂O) was labeled with Fe³⁺ or Cu²⁺ by addition of 20 μL of fresh FeCl₃ (10 mg/mL) in PBS (pH=7.4) or 20 μL of CuCl₂ (10 mg/mL) in buffer solution of pH=5.5 followed by a 1 h incubation at 40° C. The labeled complexes were then purified by a PD-10 column. The products were washed out by PBS and passed through a 0.22-μm Millipore filter into a sterile vial for in vitro and animal experiments. The Fe³⁺ and Cu²⁺ concentrations of MNPs were measured by inductively coupled plasma-mass spectrometry JCP-MS) analysis. The stability of metal ion-chelated MNPs were studied by incubating those MNPs in mouse serum at 37° C. Those MNPs were placed in dialysis tube (MWCO 10K) with magnetic stirring, dialysis against 10 ml mouse serum. At a certain time, dialysate was removed for ICP-MS analysis and replaced with fresh mouse serum.

### Characterization of MNPs.

FT-IR spectra were measured in a transmission mode on a Bio-Rad FT-IR spectrophotometer (Model FTS135) under ambient conditions. Samples of pristine melanin granules and functionalized MNPs were ground with KBr and then compressed into pellets. Transmission electron microscopy (TEM) images were recorded on a JEOL 2010 transmission electron microscope at an accelerating voltage of 100 kV. The TEM specimens were made by placing a drop of the nanoparticle aqueous solution on a carbon-coated copper grid. The hydrodynamic sizes of the MNPs were determined by dynamic light scattering (DLS) using a 90 Plus particle size analyzer (Malvern, Zetasizer Nano ZS90). Zeta potentials were measured using a zeta potential analyzer (Malvern, Zetasizer Nano ZS90). The ¹H-NMR spectra were recorded at 20° C. on a 400 MHz NMR spectrometer (Bruker), using D₂O as solvent.

### 64Cu²⁺ Radiolabeling.

The MNPs with or without Fe³⁺ were further radiolabeled with ⁶⁴Cu²⁺ by addition of 1-1.5 mCi of ⁶⁴CuCl₂ in 0.1 N NaOAc (pH 5.5) buffer followed by 1 h incubation at 40° C. The radiolabeled MNPs were then purified by a PD-10 column (GE Healthcare, Piscataway, N.J., USA). The product was washed out by PBS and passed through a 0.22-μm Millipore filter into a sterile vial for in vitro and animal experiments. The investigation of the radiolabeling stability of MNPs is similar to the metal ion-chelated MNPs except that the detector ICP-MS was replaced by PerkinElmer 1470 automatic gamma-counter for counting radioactivity.

### Cell Viability.

In vitro cytotoxicity of MNPs was determined in NIH-3T3 cells by the MTT assay. NIH-3T3 cells were incubated on 96-well plate in DMEM medium containing 10% FBS and 1% penicillin/streptomycin at 37° C. in 5% CO₂ humidified atmosphere for 24 h and 0.5×10⁴ cells were seeded per well. Cells were then cultured in the medium supplemented with indicated doses of different MNPs for 24 h. The final concentrations of MNPs in the culture medium were fixed at 0.002, 0.02, and 0.2 mg/mL in the experiment. Addition of 10 μL of MTT (0.5 mg/mL) solution to each well and incubation for 3 h at 37° C. was followed to produce formazan crystals. Then, the supernatant was removed and the products were lysed with 200 μL of DMSO. The absorbance value was recorded at 590 nm using a microplate reader. The absorbance of the untreated cells was used as a control and its absorbance was as the reference value for calculating 100% cellular viability.

### In Vitro Cell Uptake.

U87MG cells (1×10⁵ per well) were seeded in 12-well tissue culture plates and allowed to attach overnight. The cells were washed twice with serum-free DMEM and incubated with the ⁶⁴Cu-labeled MNPs (2 μCi per well, final concentration approximately 6 nM) in 400 μL of serum-free DMEM at 37° C. The specific binding of the probes with U87MG cells was determined by co-incubation with RGD (30 μg per well). After 1, 2, and 4 h, the cells were washed three times with cold PBS and lysed with the addition of 200 μL of 0.2 M NaOH. The radioactivity of all fractions was counted using a PerkinElmer 1470 automatic gamma-counter. The uptake (counts per minute) was expressed as the percentage of added radioactivity.

For MRI analysis, U87MG cells (4×10⁵ per well) were seeded in 6-well tissue culture plates and allowed to attach overnight. The cells were washed twice with serum-free DMEM and incubated with Fe-chelated MNPs (0.1, 0.2. 0.4 mg/mL) in 400 μL of serum-free DMEM at 37° C. After 4 h, the cells were washed three times with cold PBS and collected. The agarose based phantoms were prepared using the 300 μL of PCR tubes. The bottom of the tubes was filled with 1% UltraPure™ agarose gel in distilled water. After being cooled down, the collected cells (100 μL, 10 million/mL) incubated with different concentrations of MNPs suspended in 1% agarose were filled into the middle part of the tubes, and then the tops of the tubes were filled with 1% agarose. T1 MRI was performed at the Small Animal Imaging Facility at Stanford University using a Siemens 1.0 T instrument. The imaging protocol consisted of localizer and axial T1-weighted fast spin echo (FSE) sequence with the following parameters: repetition time (TR): 700 ms; echo time (TE): 5.5 ms; field of view (FOV): 3.0×3.0; matrix size: 256×256; slice thickness: 1 mm. Image analysis was performed using ImageJ. The contrast was adjusted and regions of interest (ROIs) were drawn over the samples, and the signal of ROIs was then measured.

### PAI Analysis of Phantom.

For studying the PAI properties of MNPs, a cuboid container was half filled with 1% agarose gel to half depth. Different concentrations of MNPs aqueous solutions ranging from 0.625 μM to 20 μM were filled into polyethylene capillaries and then the capillaries were laid on the surface of solidified agarose gel. The capillaries were further covered with thin 1% agarose gel to make the surface smooth. For the particle's sensitivity in living body, MNPs aqueous solutions with different concentrations from 5 μM to 80 μM were mixed with matrigel at 0° C. and then subcutaneously injected on the lower back of mice. The PAIs of the mixtures were collected after they were solidified.

The Vevo LAZR PAI System (VisualSonics Inc., Toronto, Canada) with a laser at excitation wavelength of 680 nm and a focal depth of 10 mm was used to acquire photoacoustic and ultrasound images. Image analysis was carried out using ImageJ. Briefly, quantification analysis was performed on the PAI images. All slices of a sample were stacked by Z-Project with the maximum intensity, and ROIs were drawn over the cell sample on the stacked PAI images. The PAI signal intensity was then measured using the ROIs manager tool.

### MRI Analysis of Phantom.

MRI experiments were performed at 25° C. in a magnetic resonance (MR) scanner (Siemens 1.0 T). To simulate the biological environment, agarose gel, prepared in 300 μL of the PCR tube using secondary distilled water as the solvent for dissolving the agarose, was used to demonstrate the magnetic signal. The bottom of the tube was firstly covered with a layer of 1% agarose gel. When agarose gel was cooled, the mixtures of MNPs and aqueous solution of agarose (ratio 1:1 by volume) with iron concentrations at 62.5, 125, 250, 500, and 1000 μM Fe (amount to 1.25, 2.5, 5, 10, 20 μM MNP), were then filled into the intermediate portion of the PCR tube respectively while the sample was hot. After cooling, another 1% agarose gel was covered on the top layer of the cube. The tubes were placed into the MR scanner and a number of MR sequences were run, spin-echo for R₁ determination (TR: 50-3000 ms; TE: 5.5 ms, flip angle 300; FOV: 6×6, matrix: 256×256; slice thickness: 1 mm). The luminance values of the resulting image were obtained through the Image J software processing, thereby calculating the R₁ value.

For measurement the MNPs' detection sensitivity in living subject, Fe-chelated MNPs aqueous solution with different concentrations from 1.25 μM to 20 μM were mixed with matrigel at 0° C. and then subcutaneous injected on the lower back of mice. The MRIs of the mixtures were collected after they were solidified.

### Subcutaneous Tumor Models.

All animal experiments were performed in compliance with the Guidelines for the Care and Use of Research Animals established by the Stanford University Animal Studies Committee. Female athymic nude mice (nu/nu) in 4-6 weeks old were obtained from the Charles River Laboratories (Boston, Mass., USA) and kept under sterile conditions. U87MG cells suspended in 100 μL of PBS were inoculated subcutaneously in the shoulder of nude mice. When the tumors reached 0.5-0.8 cm in diameter, the tumor bearing mice were subjected to in vivo multimodality imaging (PAI, MRI and PET) and biodistribution studies.

### PAI and MRI of Tumor Bearing Mice.

Mice bearing tumor (U87MG) were anesthetized with 2% isoflurane in oxygen and placed with lateral position. MRI was performed using the same instrument, protocols and conditions as in the phantom MRI study. Imaging analysis was performed using the OsiriX software. The contrast was adjusted and ROIs were drawn over the tumor and muscle. T1 value of ROIs was then measured, and the ratio of tumor/muscle was calculated. PAI was carried out using the same Vevo LAZR PAI System as the in vitro study. Similarly, image analysis was carried out using ImageJ, and quantification analysis was performed on the PAI images.

### Small-Animal PET.

Small animal PET imaging of tumor-bearing mice was performed on a Siemens Inveon microPET-CT. Mice bearing U87-MG tumors were injected with ⁶⁴Cu-labeled MNPs (110.0±5.0 μCi) via the tail vein. At different times after injection (2, 4 and 24 h), the mice were anesthetized with 2% isoflurane and placed prone near the center of the FOV of the scanner. Three-minute static scans were obtained. All the small animal PET images were reconstructed by two dimensional ordered-subsets expectation maximization (OSEM) algorithm. No background correction was performed. The radioactivity uptake in the tumor and normal tissues was calculated using a region of interest (ROI) drawn over the whole organ region and expressed as a percentage of the injected radioactive dose per gram of tissue (% ID/g).

TABLE 1
The data of hydrodynamic sizes and zeta
potentials of MNPs in aqueous solution.

| MNP | Diameter (nm) | Zeta potential (mV) |
|---|---|---|
| PWS-MNP | 4.5 ± 0.3 | −22.5 ± 1.2 |
| PEG-MNP | 7.0 ± 0.6 | −4.5 ± 0.6 |
| RGD-PEG-MNP | 8.7 ± 0.9 | −4.1 ± 0.1 |
| Fe-PEG-MNP | 9.0 ± 0.6 | +2.1 ± 0.4 |
| Fe-RGD-PEG-MNP | 11.2 ± 1.3 | +0.2 ± 0.2 |

FIG. 2.7A illustrates zeta potentials of PWS-MNP (top) and PEG-MNP (bottom). FIG. 2.7B illustrates hydrodynamic size distribution graphs of PWS-MNP (top) and PEG-MNP (bottom).

FIG. 2.8A illustrates FT-IR spectra of pristine melanin granule, PWS-MNP and PEG-MNP. In FT-IR spectra, PEG-MNP showed characteristic absorption peaks of PEG at 2880 cm⁻¹ (alkyl C—H stretching) and 1110 cm⁻¹ (C—O—C stretching). FIG. 2.8B illustrates ¹H NMR spectra of PWS-MNP and PEG-MNP in D₂O ¹H NMR further confirmed the existence of PEG on the MNP. A new peak at 3.5 ppm attributing to PEG (—OCH₂CH₂O—) appeared in the ¹H NMR of PEG-MNP.

FIG. 2.9A illustrates a plot of the relationship between the weight ratio of the product composition (PEG:PWS-MNP) and the feed ratio (W_{PEG} W_{PWS-MNP}). FIG. 2.9A illustrates the saturation weight ratio of PEG to PWS-MNP, which is about 1.2:1. FIG. 2.9B illustrates the UV-vis-NIR absorption spectra of PWS-MNP and PEG-MNP. The combined with the molecular weight of PWS-MNP, the number of PEG chain on every PEG-MNP is about 10.

FIG. 2.10A illustrates the hydrodynamic size distribution graphs of RGD-PEG-MNP (top), Fe-PEG-MNP (middle), and Fe-RGD-PEG-MNP (bottom). FIG. 2.10B illustrates the zeta potentials of RGD-PEG-MNP (top), Fe-PEG-MNP (middle), and Fe-RGD-PEG-MNP (bottom).

FIG. 11, from left to right, illustrates pictures of (1) 1 mL of 20 μM PWS-MNP aqueous solution after adding 0.2 mL of 10 mM FeCl₃, (2) 1 mL of 20 μM PWS-MNP aqueous solution after adding 0.2 mL of 10 mM CuCl₂, (3) 1 mL of 20 μM PEG-MNP aqueous solution after adding 0.2 mL of 10 mM FeCl₃, (4) 1 mL of 20 μM PEG-MNP aqueous solution after adding 0.2 mL of 10 mM CuCl₂. It was showed that PEG-encapsulation will hamper the formation of precipitation of MNPs after adding metal ions.

FIG. 12 illustrates photobleaching of MNPs. RGD-PEG-MNP and PEG-MNP samples (n=3 for each) were exposed to increasing durations of 680 nm laser light, at power density of 8 mJ/cm². After 60 min of laser exposure, the optical absorption of all the MNPs was reduced by ˜3%.

FIG. 13 illustrates MTT assay using NIH-3T3 cells with MNP concentration 0.2, 0.5, 1 and 2 μM after 24 h incubation at 37° C.

FIG. 14 illustrates T₁-weighted MRI images (1.0 T, spin-echo sequence: repetition time TR=700 ms, echo time TE=5.5 ms) of Fe-RGD-PEG-MNP with different concentration. FIG. 15 illustrates in vitro mouse serum and PBS stability study of ⁶⁴Cu-RGD-PEG-MNP and ⁶⁴Cu-PEG-MNP. After 24 h incubation, only ˜3% ⁶⁴Cu was released from the MNPs

### REFERENCES

1. Tan, S. J., Campolongo, M. J., Luo, D. & Cheng, W. L. Building plasmonic nanostructures with DNA. Nature Nanotechnol. 6, 268-276 (2011).
2. Rinker, S., Ke, Y., Liu, Y., Chhabra, R. & Yan, H. Self-assembled DNA nanostructures for distance-dependent multivalent ligand-protein binding. Nature Nanotechnol. 3, 418-422 (2008).
3. Ke, Y., Ong, L. L., Shih, W. M. & Yin, P. Three-dimensional structures self-assembled from DNA bricks. Science 338, 1177-1183 (2012).
4. Schnepp, Z. Biopolymers as a flexible resource for nanochemistry. Angew. Chem. Int. Ed. 52, 1096-1108 (2012).
5. Godin, B., Tasciotti, E., Liu, X., Serda, R. E. & Ferrari, M. Multistage nanovectors: from concept to novel imaging contrast agents and therapeutics. Accounts Chem. Res. 44, 10979-10989 (2011).
6. Parodi, A. et al. Synthetic nanoparticles functionalized with biomimetic leukocyte membranes possess cell-like functions. Nature Nanotechnol. 8, 61-68 (2012).
7. Weissleder, R. & Pittet, M. J. Imaging in the era of molecular oncology. Nature 452, 580-589 (2008).
8. Tassa, C., Shaw, S. Y. & Weissleder, R. Dextran-coated iron oxide nanoparticles: a versatile platform for targeted molecular imaging, molecular diagnostics, and therapy. Acc. Chem. Res. 44, 842-852 (2008).
9. Michalet, X. et al. Quantum dots for live cells, in vivo imaging, and diagnostics. Science 307, 538-544 (2005).
10. Zhou, M. et al. A chelator-free multifunctional [⁶⁴Cu]CuS nanoparticle platform for simultaneous micro-PET/CT imaging and photothermal ablation therapy. J. Am. Chem. Soc. 132, 15351-15358 (2010).
11. Wang, Y. C. et al. Radioluminescent gold nanocages with controlled radioactivity for real-time in vivo imaging. Nano Lett. 13, 581-585 (2013).
12. Luo, S. L., Zhang, E. L., Su, Y. P., Cheng, T. M. & Shi, C. M. A review of NIR dyes in cancer targeting and imaging. Biomaterials 32, 7127-7138 (2011).
13. J. Seo, et al. In vivo biodistribution and small animal PET of ⁶⁴Cu-labeled antimicrobial peptoids. Bioconjugate Chem. 23, 1069-1079 (2012).
14. Werner, E. J., Datta, A., Jocher, C. J. & Raymond, K. N. High-relaxivity MRI contrast agents: where coordination chemistry meets medical imaging. Angew. Chem. Int. Ed. 47, 8568-8580 (2008).
15. Jimbow, K. et al. Characterization of melanogenesis and morphogenesis of melanosomes by physicochemical properties of melanin and melanosomes in malignant melanoma. Cancer Res. 44, 1128-1134 (1984).
16. Prota, G. Melanins, melanogenesis and melanocytes: looking at their functional significance from the chemist's viewpoint. Pigment Cell Res. 13, 283-293 (2000).
17. Iozumi, K., Hoganson, G. E., Pennella, R., Everett, M. A. & Fuller, B. B. Role of tyrosinase as the determinant of pigmentation in cultured human melanocytes. J. Invest. Dermatol. 100, 806-811 (1993).
18. Seagle, B.-L. L. et al. Melanin photoprotection in the human retinal pigment epithelium and its correlation with light-induced cell apoptosis. P. Natl. Acad. Sci. USA 102, 8978-8983 (2005).
19. Lu, S., Fan, Q.-L., Chua, S.-J. & Huang, W. Synthesis of conjugated-ionic block copolymers by controlled radical polymerization. Macromolecules 36, 304-310 (2003).
20. Moghimi S. M., Hunter A. C. & Murray J. C. Long-circulating and targetspecific nanoparticles: theory to practice. Pharmacol. Rev. 53, 283-318 (2001).
21. Lee, H. et al. Mussel-inspired surface chemistry for multifunctional coatings. Science 318, 426-430 (2007).
22. Zerda, A. D. L. et al. Carbon nanotubes as photoacoustic molecular imaging agents in living mice. Nature Nanotechnol. 3, 557-562 (2008).
23. Zerda, A. D. L. et al. Family of enhanced photoacoustic imaging agents for high-sensitivity and multiplexing studies in living mice. ACS Nano 6, 4694-4701 (2012).
24. Cheng, Z. et al. ⁶⁴Cu-labeled affibody molecules for imaging of HER2 expressing tumors, Mol. Imaging. Biol. 12, 316-324 (2010).
25. Nielsen, C. H. et al. PET Imaging of tumor neovascularization in a transgenic mouse model using a novel ⁶⁴Cu-DOTA-Knottin peptide. Cancer Res. 70, 9022-9030 (2010).
26. Hoppmann, S. et al. Radiolabeled affibody-albumin bioconjugates for HER2 positive cancer targeting. Bioconjug. Chem. 22, 413-421 (2011).
27. Lovell, J. F. et al. Porphysome nanovesicles generated by porphyrin bilayers for use as multimodal biophotonic contrast agents. Nature Mater. 10, 324-332 (2011).
28. Huynh, E. et al. Porphyrin shell microbubbles with intrinsic ultrasound and photoacoustic properties. J. Am. Chem. Soc. 134, 16464-16467 (2012).
29. Liu, T. W. et al. Inherently multimodal nanoparticle-driven tracking and real-time delineation of orthotopic prostate tumors and micrometastases. ACS Nano 7, 4221-4232 (2013).
30. Simon, J. D., Hong, L. & Peles, D. N. Insights into melanosomes and melanin from some interesting spatial and temporal properties. J. Phys. Chem. B 112, 13201-13217 (2008).
31. Cheng, Z., Mahmood, A., Li, H., Davison, A. & Jones, A. G. [⁹⁹ mTcOAADT]-(CH₂)₂—NEt₂: a potential small-molecule single-photon emission computed tomography probe for imaging metastatic melanoma. Cancer Res. 65, 4979-4986 (2005).
32. Cheng, Z. et al. Small-animal PET of melanocortin 1 receptor expression using a ¹⁸F-labeled alpha-melanocyte-stimulating hormone analog. J. Nucl. Med. 48, 987-994 (2007).
33. Ren, G. et al. Melanin-targeted preclinical PET imaging of melanoma metastasis. J. Nucl. Med. 50, 1692-1699 (2009).
34. Oh, J. T. et al. Three-dimensional imaging of skin melanoma in vivo by dual-wavelength photoacoustic microscopy. J. Biomed. Opt. 11, 34032 (2006).
35. Wang, Y. et al. Fiber-laser-based photoacoustic microscopy and melanoma cell detection. J. Biomed. Opt. 16, 011014 (2011).
36. Hong, L. & Simon, J. D. Current understanding of the binding sites, capacity, affinity, and biological significance of metals in melanin. J. Phys. Chem. B 111, 7938-7947 (2007).
37. Samokhvalov, A., Liu, Y. & Simon, J. D. Characterization of the Fe(III)-bindin site in Sepia eumelanin by resonance Raman confocal microspectroscopy. Photochem. Photobiol. 80, 84-88 (2004).
38. Liu, Y. et al. Ion-exchange and adsorption of Fe(III) by Sepia melanin. Pigment Cell Res. 17, 262-269 (2004).
39. Woodruff, W. W. J., Djang, W. T., McLendon, R. E., Heinz, E. R. & Voorhees, D. R. Intracerebral malignant melanoma: high-field-strength MR imaging. Radiology 165, 209-213 (1987).
40. Ginat, D. T. & Meyers, S. P. Intracranial lesions with high signal intensity on T1-weighted MR images: differential diagnosis. Radiographics 32, 499-516 (2012).
41. Krumholz, A. et al. Photoacoustic imaging of gene expression using tyrosinase as a reporter gene. J. Biomed. Opt. 16, 080503 (2011).
42. Qin, C. X. et al. Tyrosinase as a multifunctional reporter gene for Photoacoustic/MRI/PET triple modality molecular imaging. Sci. Rep. 3, 1490 (2013).
43. Stritzker, J. et al. Vaccinia virus-mediated melanin production allows MR and optoacoustic deep tissue imaging and laser-induced thermotherapy of cancer. P. Natl. Acad. Sci. USA 110, 3316-3320 (2013).
44. Hong, S. et al. Non-covalent self-assembly and covalent polymerization co-contribute to polydopamine formation. Adv. Funct. Mater. 22, 4711-4717 (2012).
45. Ceylan, H. et al. Mussel inspired dynamic cross-linking of self-healing peptide nanofiber network. Adv. Funct. Mater. 23, 2081-2090 (2013).
46. INGS, R. M. J. The melanin binding of drugs and its implications. Drug Metab. Rev. 15, 1183-1212 (1984).

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to significant figures of the numerical value. In addition, the phrase “about ‘x’ to ‘y’” includes “about ‘x’ to about ‘y’”.

It should be emphasized that the above-described embodiments of the present disclosure are merely possible examples of implementations, and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

We claim at least the following:

1. A composition, comprising:
a water soluble polyethylene glycol (PEG)-polymeric melanin (PEG-melanin) nanoparticle, wherein the PEG is attached to the surface of the polymeric melanin nanoparticle core, wherein the polymeric melanin nanoparticle core has a diameter of about 3 to 10 nm.

2. The composition of claim 1, further comprising a MRI agent.

3. The composition of claim 2, wherein the MRI agent is attached to the PEG-melanin nanoparticle using a chelating agent bonded to the PEG-melanin nanoparticle.

4. The composition of claim 3, wherein the chelating agent is bonded to the PEG of the PEG-melanin nanoparticle.

5. The composition of claim 1, further comprising a PET or SPECT agent.

6. The composition of claim 5, wherein the PET or SPECT agent is attached to the PEG-melanin nanoparticle using a chelating agent bonded to the PEG-melanin nanoparticle.

7. The composition of claim 6, wherein the chelating agent is bonded to the PEG of the PEG-melanin nanoparticle.

8. The composition of claim 1, further comprising a MRI agent and a PET or SPECT agent, wherein the MRI agent and the PET or SPECT agent are bonded to the PEG of the PEG-melanin nanoparticle.

9. The composition of claim 1, wherein the polymeric melanin nanoparticle core has a diameter of about 3 to 6 nm.

10. The composition of claim 1, wherein the PEG-melanin nanoparticle has a diameter of about 4 to 20 nm.

11. A method of imaging a disease, comprising:
exposing a subject to an imaging device, wherein a PEG-melanin nanoparticle is introduced to a subject, wherein PEG-melanin nanoparticle is a water soluble PEG-melanin nanoparticle, wherein the PEG is attached to the surface of the polymeric melanin nanoparticle core, wherein the polymeric melanin nanoparticle core has a diameter of about 3 to 10 nm; and
detecting the PEG-melanin nanoparticle, wherein the location of the PEG-melanin nanoparticle correlates to the location of the disease.

12. The method of claim 11, wherein the detection is conducted in vitro or in vivo.

13. The method of claim 11, wherein the imaging device is selected from a photoacoustic device, MRI imaging device, a PET imaging device, or a combination thereof.

14. The method of claim 11, wherein the imaging device is a photoacoustic device, and
detecting the PEG-melanin nanoparticle including a photoacoustic signal associated with the PEG-melanin nanoparticle, wherein the photoacoustic signal correlates to the position of the disease within the subject.

15. The method of claim 11, wherein the disease is a melanin related disease, cancer, tumor, or precancerous cell.

16. A pharmaceutical composition, comprising:
a pharmaceutical carrier and an effective amount of a PEG-melanin nanoparticle,
wherein PEG-melanin nanoparticle is a water soluble PEG-melanin nanoparticle, wherein the PEG is attached to the surface of the polymeric melanin nanoparticle core, wherein the polymeric melanin nanoparticle core has a diameter of about 3 to 10 nm.

17. The pharmaceutical composition of claim 16, wherein the MRI agent is attached to the PEG-melanin nanoparticle using a chelating agent bonded to the PEG-melanin nanoparticle.

18. The pharmaceutical composition of claim 16, wherein the PET or SPECT agent is attached to the PEG-melanin nanoparticle using a chelating agent bonded to the PEG-melanin nanoparticle.

19. The pharmaceutical composition of claim 16, further comprising a MRI agent and a PET or SPECT agent, wherein the MRI agent and the PET or SPECT agent are bonded to the PEG of the PEG-melanin nanoparticle.

20. A method of making a PEG-melanin nanoparticle, comprising:
dissolving melanin in a basic aqueous solution;
adjusting the pH to about 7 under sonication to form melanin nanoparticles;
adjusting the pH to about 10; and
adding PEG precursor compounds to the solution to form PEG-melanin nanoparticles, wherein the PEG is attached to the surface of a polymeric melanin nanoparticle core, wherein the polymeric melanin nanoparticle core has a diameter of about 3 to 10 nm.
